(19) United States
(12) Patent Application Publication
(10) Pub. No.: US 2016/0367685 A1
(43) Pub. Date: Dec. 22, 2016

(54) DUPA-INDENOISOQUINOLINE CONJUGATES

(71) Applicant: PURDUE RESEARCH FOUNDATION, West Lafayette, IN (US)

(72) Inventors: Mark S. Cushman, West Lafayette, IN (US); Phillip S. Low, West Lafayette, IN (US); Trung X. Nguyen, West Lafayette, IN (US)

(21) Appl. No.: 15/035,157

(22) PCT Filed: Nov. 5, 2014
(86) PCT No.: PCT/US14/64127

§ 371 Date: May 6, 2016

Related U.S. Application Data
(60) Provisional application No. 61/900,800, filed on Nov. 6, 2013.

(51) Int. Cl. A61K 47/48; A61K 31/473

(52) U.S. Cl. CPC A61K 47/48038; A61K 47/48338; A61K 31/473

(57) ABSTRACT

A targeting ligand-cytotoxic drug conjugate, for example, a DUPA-Indenoisoquinoline conjugate, is useful for treating cancers, e.g., prostate cancer.

### GOVERNMENT INTEREST STATEMENT

This invention was made with government support under CA089566, awarded by the National Institutes of Health. The government has certain rights in the invention.

### TECHNICAL FIELD

The invention relates to targeting ligand-cytotoxic drug conjugates, e.g., DUPA-indenoisoquinoline conjugates, which are useful for treating cancers, e.g., prostate cancer.

### BACKGROUND OF THE INVENTION

Prostate cancer is the second leading cause of cancer death of men in the United States (following lung cancer as number one) with an estimated new 240,890 cases in 2011 and 33,720 deaths (Siegel, et al, CA Cancer J. Clin. 2011, 61, 212-236). The current types of treatment include hormonal therapy and chemotherapy, but both come with disappointing and sometimes detrimental consequences that compromise their uses. The efficacy of chemotherapy in cancer treatment is often limited by two main factors: side toxicity and the emergence of tumor resistance (Soudy, et al., J. Med. Chem. 2013, 56, 7564-7573). Therefore there is an urgent need for developing methodologies that can selectively kill cancer cells without the usual collateral damage, and prevent tumor cells from acquiring resistance.

Most prostate cancer cells overexpress the prostate-specific membrane antigen (PSMA) with an increase of 8 to 12 folds over the normal prostate cells (O'Keefe, et al., The Prostate 2004, 58, 200-210). In addition, gene array analysis and immunohistochemistry studies revealed that PSMA is the second most up-regulated protein in prostate cancer, and the expression level rises with the aggressiveness of cancer (Wang, et al., J. Cell. Biochem. 2007, 102, 571-579). PSMA is also found to be highly overexpressed in the neovasculature of solid tumors, especially as the tumor progresses or metastasizes, while being present at low or undetectable levels in normal tissues (Ghosh, et al., J. Cell. Biochem. 2004, 91, 528-539). This difference could be taken advantage of in order to deliver non-specific cytotoxic drugs to these pathogenic cells while sparing normal cells that lack PSMA, thus improving potencies and reducing toxicities.

### SUMMARY OF THE INVENTION

The present invention features a targeting ligand-cytotoxic drug conjugate.

In one aspect, the invention features a DUPA-drug conjugate represented by formula (IA):

DUPA-Linker-RS-Drug (TA)

wherein
DUPA is a modified or unmodified 2-[3-(1,3-dicarboxypropyl)-ureido]pentanedioic acid;
Linker is a bond, a substituted or unsubstituted alkyl, a peptide, or a peptidoglycan;
Drug is a cytotoxic drug; and
RS is a release segment capable of releasing the drug within the desired cells, wherein said release segment is a carbonate segment, a carbamate segment, or an acylhydrazone segment.

In another aspect, the invention features a DUPA-Indenoisoquinoline conjugate represented by formula (IB):

DUPA-Linker-RS-Indenoisoquinoline (IB)

wherein
DUPA is a modified or unmodified 2-[3-(1,3-dicarboxypropyl)-ureido]pentanedioic acid;
Linker is a bond, a substituted or unsubstituted alkyl, a peptide, or a peptidoglycan;
Indenoisoquinoline is a substituted or unsubstituted indenoisoquinoline; and
RS is a release segment capable of releasing Indenoisoquinoline within the desired cells, wherein said release segment is a carbonate segment, a carbamate segment, or an acylhydrazone segment.

In some embodiments, the DUPA-Indenoisoquinoline conjugate is represented by formula (II):

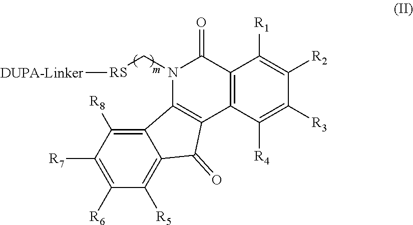

wherein
R₁, R₂, R₃, R₄, R₅, R₆, R₇, and R₈ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group;
R₁₁ and R₁₂ are each independently H or C₁₋₅ alkyl, wherein C₁₋₅ alkyl is optionally mono- or poly-substituted with substituents independently selected from halo, OH, O—C₁₋₃ alkyl, amino, C₁₋₃ alkylamino, and di-C₁₋₃ alkylamino; or R₁₁ and R₁₂, together with the nitrogen atom to which they are attached, form a 4-7 membered cycloheteroalkyl or heteroaryl; and
m is 0-5.

In some embodiments, the DUPA-Indenoisoquinoline conjugate is represented by formula (III):

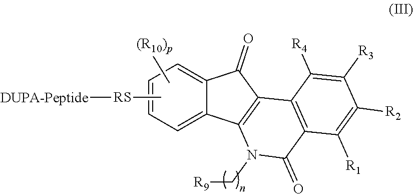

wherein
R₁, R₂, R₃, R₄, and R₁₀ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group;
R₉ is H, halo, O—C₁₋₅ alkyl, NR₁₁R₁₂, nitro, C₃₋₆ cycloalkyl, or C₃₋₈ cycloheteroalkyl;
R₁₁ and R₁₂ are each independently H or C₁₋₅ alkyl, wherein C₁₋₅ alkyl is optionally mono- or poly-substituted with substituents independently selected from halo, OH, O—C₁₋₃ alkyl, amino, C₁₋₃ alkylamino, and di-C₁₋₃ alkylamino; or R₁₁ and R₁₂, together with the nitrogen atom to which they are attached, form a 4-7 membered cycloheteroalkyl or heteroaryl;
n is 0-5; and
p is 3.

In some embodiments, the DUPA-Indenoisoquinoline conjugate is represented by formula (IV)

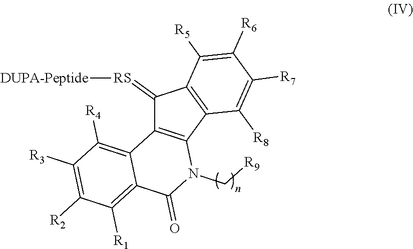

wherein
R₁, R₂, R₃, R₄, R₅, R₆, R₇, and R₈ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group;

R₉ is H, halo, O—C₁₋₅ alkyl, NR₁₁R₁₂, nitro, C₃₋₆ cycloalkyl, or C₃₋₈ cycloheteroalkyl;
R₁₁ and R₁₂ are each independently H or C₁₋₅ alkyl, wherein C₁₋₅ alkyl is optionally mono- or poly-substituted with substituents independently selected from halo, OH, O—C₁₋₃ alkyl, amino, C₁₋₃ alkylamino, and di-C₁₋₃ alkylamino; or R₁₁ and R₁₂, together with the nitrogen atom to which they are attached, form a 4-7 membered cycloheteroalkyl or heteroaryl group; and
n is 0-5.

In another aspect, the invention features a pharmaceutical composition comprising a DUPA-Indenoisoquinoline conjugate of the invention, e.g., formulas (III)-(XX), and at least one pharmaceutically acceptable carrier.

In another aspect, the invention features a method of treating cancer in a subject in need thereof, the method comprising administering to said subject a therapeutically effective amount of a DUPA-Indenoisoquinoline conjugate of the invention, e.g., formulas (III)-(XX), or a composition comprising the DUPA-Indenoisoquinoline conjugate. In some embodiments, said cancer is prostate cancer, ovarian cancer, lung cancer, or breast cancer. In certain embodiments, said cancer is prostate cancer.

In yet another aspect, the invention features a process of preparing a DUPA-Indenoisoquinoline conjugate represented by formula (IB):

DUPA-Linker-RS-Indenoisoquinoline (IB)

wherein
DUPA is a modified or unmodified 2-[3-(1,3-dicarboxypropyl)-ureido]pentanedioic acid;
Linker is a bond, a substituted or unsubstituted alkyl, a peptide, or a peptidoglycan;
Indenoisoquinoline is a substituted or unsubstituted indenoisoquinoline; and
RS is a release segment capable of releasing Indenoisoquinoline within the desired cells,
   wherein said release segment is a carbonate segment, a carbamate segment, or an acylhydrazone segment.
   the process comprising
(a) reacting a DUPA with a peptide to prepare a DUPA-peptide reagent;
(b) reacting an RS reagent with an indenoisoquinoline to prepare an RS-indenoisoquinoline compound; and
(c) reacting the DUPA-peptide reagent of step (a) with the RS-indenoisoquinoline compound of step (b) to prepare said DUPA-Indenoisoquinoline conjugate.

In some embodiments, the RS reagent is represented by formula (XXI):

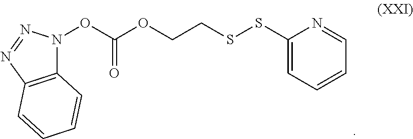 [SMILES: O=C(OCCSSc1ccccn1)On1nnc2ccccc21]

In some embodiments, the DUPA-peptide reagent is represented by formula (XXII):

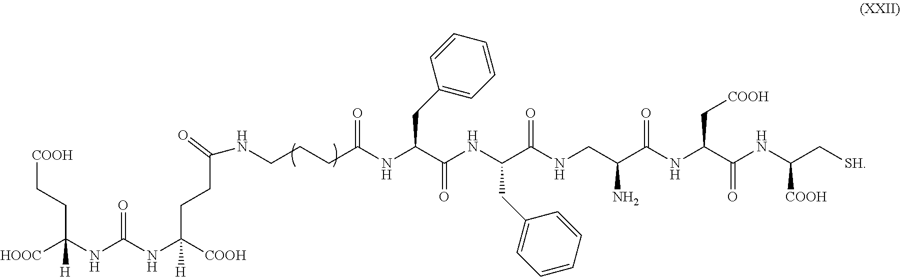

The details of one or more embodiments of the invention are set forth in the accompanying the description below. Other features, objects, and advantages of the invention will be apparent from the description and drawings, and from the claims.

### BRIEF DESCRIPTION OF THE DRAWINGS

FIG. 1 depicts a general schematic representation of a DUPA-Indenoisoquinoline conjugate.

FIG. 2 depicts a molecular model of the truncated fragment 52 bound to PSMA. The stereoview is programmed for walleyed (relaxed) viewing.

FIG. 3 depicts cytotoxicities of indenoisoquinolines 6 and 18, and their DUPA conjugates 84 and 86 in LNCaP cell cultures.

FIG. 4 illustrates the tumor volume vs. Days of therapy with DUPA conjugate 86 in animal studies with athymic nude mice bearing 22RV1 tumors. Treated=86; untreated=control; competition=86+10×28; free drug=18. Dose: 40 nmol/mouse, or 2.0 μmol/kg, IP injection, alternate days, 3 days/week for 3 weeks.

FIG. 5 illustrates the live mice vs. doses of therapy with DUPA conjugate 86 in animal studies with athymic nude mice bearing 22RV1 tumors. Treated=86; untreated=control; competition=86+10×28; free drug=18. Dose: 40 nmol/mouse, or 2.0 μmol/kg, IP injection, alternate days, 3 days/week for 3 weeks.

FIG. 6 illustrates the average body weight vs. days of therapy with DUPA conjugate 86 in animal studies with athymic nude mice bearing 22RV1 tumors. Treated=86; untreated=control; competition=86+10×28; free drug=18. Dose: 40 nmol/mouse, or 2.0 μmol/kg, IP injection, alternate days, 3 days/week for 3 weeks. Note: After the 26^{th} day in the base drug group, the data points represent the weight of only one mouse, whereas the other four mice died of drug cytotoxicity.

FIGS. 7A and 7B depict test results of MC-7-70 (compound 18) and its DUPA conjugate in LNCap cell lines. FIG. 7A: therapy of DUPA-MC-7-70 in LNCap tumor bearing mice; and FIG. 7B: weight loss of mice during treatment with DUPA-MC-7-70.

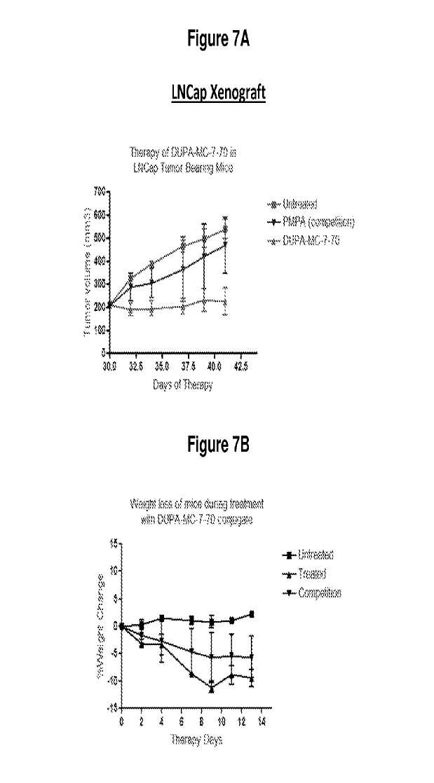

FIGS. 8A-8C depict test results of DUPA-MC-7-70 (MC-7-70 is compound 18). FIG. 8A: therapy of DUPA-MC-7-70 in LNCap Tumor Bearing Mice; FIG. 8B: IC₅₀ of DUPA-MC-7-70 in LNCaP cells 2 h and 24 h incubation; and FIG. 8C: weight loss of mice during treatment with DUPA-MC-7-70 conjugate.

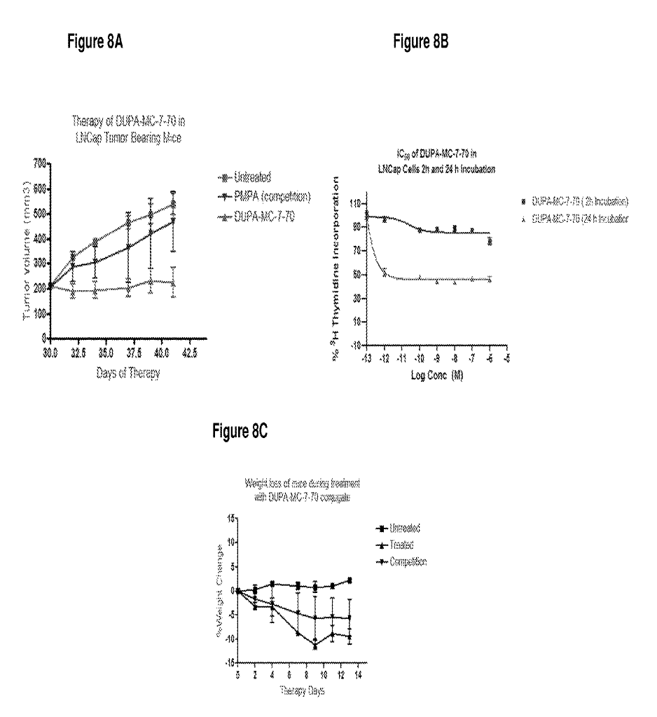

FIG. 9 depicts test results for the compounds or conjugates of the invention.

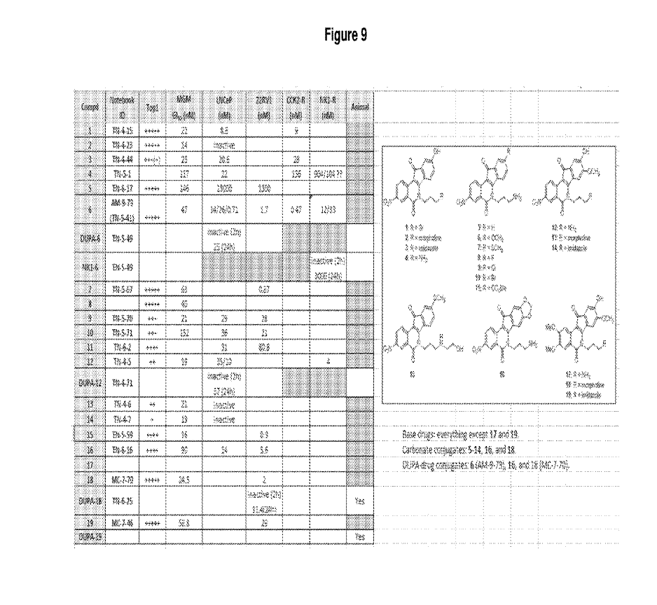

FIGS. 10A and 10B depict dose-response ³H-thymidine incorporation assays of free drug 18 (FIG. 10A) and DUPA conjugate 86 (FIG. 10B) on the survival of human 22RV1 cell lines after indicated incubation times.

FIGS. 11A and 11B depict the molecular model of the DUPA-indenoisoquinoline conjugate 86 bound to PSMA. The stereoview is programmed for wall-eyed (relaxed) viewing. FIG. 11A: ligand, space filling model; protein, stick model. FIG. 11B: ligand, stick model; protein, ribbon and space filling model.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The invention provides a targeting ligand-cytotoxic drug conjugate.

In some embodiments, the invention features a DUPA-drug conjugate represented by formula (IA):

DUPA-Linker-RS-Drug (IA)

wherein
DUPA is a modified or unmodified 2-[3-(1,3-dicarboxypropyl)-ureido]pentanedioic acid;
Linker is a bond, a substituted or unsubstituted alkyl, a peptide, or a peptidoglycan;
Drug is a cytotoxic drug; and
RS is a release segment capable of releasing the drug within the desired cells, wherein said release segment is a carbonate segment, a carbamate segment, or an acylhydrazone segment.

In some embodiments, this invention is directed to a DUPA-Indenoisoquinoline conjugate represented by (IB):

DUPA-Linker-RS-Indenoisoquinoline (IB)

wherein
DUPA is a modified or unmodified 2-[3-(1,3-dicarboxypropyl)-ureido]pentanedioic acid;
Linker is a bond, a substituted or unsubstituted alkyl, a peptide, or a peptidoglycan;
Indenoisoquinoline is a substituted or unsubstituted indenoisoquinoline; and
RS is a release segment capable of releasing Indenoisoquinoline within the desired cells,
   wherein said release segment is a carbonate segment, a carbamate segment, or an acylhydrazone segment.

In some embodiments, the Linker is a peptide.

In some embodiments, the Indenoisoquinoline is an indenoisoquinoline as described herein. In other embodiments, the Indenoisoquinoline is an indenoisoquinoline known in the art. The indenoisoquinoline described herein can be further substituted with a group selected from halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, and C₃₋₈ cycloheteroalkyl; or two adjacent O—C₁₋₃ alkyl groups, together with the atoms to which they are attached, form a 5-7 membered cycloheteroalkyl group.

In some embodiments, said DUPA-Indenoisoquinoline conjugate is represented by formula (II)

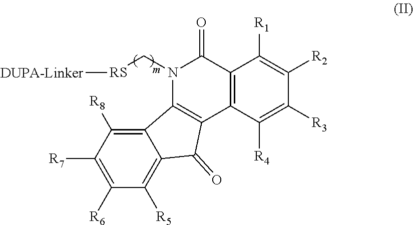

wherein
R₁, R₂, R₃, R₄, R₅, R₆, R₇, and R₈ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group;
R₁₁ and R₁₂ are each independently H or C₁₋₅ alkyl, wherein C₁₋₅ alkyl is optionally mono- or poly-substituted with substituents independently selected from halo, OH, O—C₁₋₃ alkyl, amino, C₁₋₃ alkylamino, and di-C₁₋₃ alkylamino; or R₁₁ and R₁₂, together with the nitrogen atom to which they are attached, form a 4-7 membered cycloheteroalkyl or heteroaryl; and
m is 0-5.

In other embodiments, m is 1. In certain embodiments, m is 2. In some embodiments, m is 3. In some embodiments, m is 4. In other embodiments, m is 5. In some embodiments, m is 2-4.

In some embodiments, the DUPA-Indenoisoquinoline conjugate is represented by formula (V)

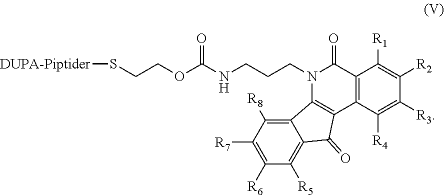

In some embodiments, R₁, R₂, R₃, R₄, R₅, R₆, R₇, and R₈ are each independently H, halo, nitro, C₁₋₅ alkyl, O—C₁₋₃ alkyl, cyano, C₁₋₃ haloalkyl, S—C₁₋₃ alkyl, SO₂R₁₁, or (CO)OR₁₁; or two adjacent O—C₁₋₃ alkyl groups, together with the atoms to which they are attached, form a 5-7 membered cycloheteroalkyl group.

In some embodiments, R₁, R₄, R₅, and R₈ are each H.

In some embodiments, R₂, R₃, R₆, and R₇ are each H, nitro, OH, OCH₃, cyano, C₁₋₃ haloalkyl. In some embodiments, R₂, R₃, R₆, and R₇ are each H, cyano, or C₁₋₃ haloalkyl. In other embodiments, R₂, R₃, R₆, and R₇ are each H or cyano. In some embodiments, R₂, R₃, R₆, and R₇ are each H or C₁₋₃ haloalkyl.

In some embodiments, R₂, R₃, R₆, and R₇ are each H, nitro, OH, or OCH₃. In some embodiments, R₂, R₃, R₆, and R₇ are each nitro, OH, or OCH₃.

In other embodiments, R₂, R₃, R₆, and R₇ are each independently H, halo, SCH₃, SO₂CH₃, or CO₂CH₃. In some embodiments, R₂, R₃, R₆, and R₇ are each independently H, halo, SCH₃, or CO₂CH₃.

In some embodiments, R₂, R₃, R₆, and R₇ are each OH or OCH₃.

In some embodiments, R₂ is nitro. In other embodiments, R₆ is OCH₃.

In some embodiments, R₆ and R₇, together with the atoms to which they are attached, form a 5-7 membered cycloheteroalkyl group. In certain embodiments, R₆ and R₇ form methylenedioxy.

In some embodiments, R₂, R₃, R₆, and R₇ are each independently H, halo, SCH₃, SO₂CH₃, or CO₂CH₃. In some embodiments, R₂, R₃, R₆, and R₇ are each independently H, halo, SCH₃, or CO₂CH₃. In other embodiments, R₆ is halo. In certain embodiments, R₆ is fluoro.

In some embodiments, the DUPA-Indenoisoquinoline conjugate is represented by formula (III)

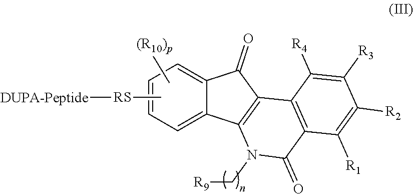

wherein
R₁, R₂, R₃, R₄, and R₁₀ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group;
R₉ is H, halo, O—C₁₋₅ alkyl, NR₁₁R₁₂, nitro, C₃₋₆ cycloalkyl, or C₃₋₈ cycloheteroalkyl;
R₁₁ and R₁₂ are each independently H or C₁₋₅ alkyl, wherein C₁₋₅ alkyl is optionally mono- or poly-substituted with substituents independently selected from halo, OH, O—C₁₋₃ alkyl, amino, C₁₋₃ alkylamino, and di-C₁₋₃ alkylamino; or R₁₁ and R₁₂, together with the nitrogen atom to which they are attached, form a 4-7 membered cycloheteroalkyl or heteroaryl;
n is 0-5; and
p is 3.

In some embodiments, n is 2-4. In other embodiments, n is 3. In other embodiments, n is 1.

In some embodiments, the DUPA-Indenoisoquinoline conjugate is represented by formula (VI)

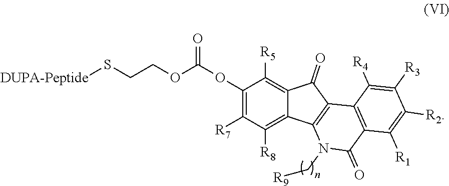

In some embodiments, R₁, R₂, R₃, R₄, R₅, R₇, and R₈ are each independently H, halo, nitro, C₁₋₅ alkyl, O—C₁₋₃ alkyl, cyano, C₁₋₃ haloalkyl, S—C₁₋₃ alkyl, SO₂R₁₁, or (CO)OR₁₁; or two adjacent O—C₁₋₃ alkyl groups, together with the atoms to which they are attached, form a 5-7 membered cycloheteroalkyl group.

In some embodiments, R₁, R₄, R₅, and R₈ are each H.

In some embodiments, R₂, R₃, R₆, and R₇ are each H, nitro, OH, OCH₃, cyano, C₁₋₃ haloalkyl. In some embodiments, R₂, R₃, R₆, and R₇ are each H, cyano, or C₁₋₃ haloalkyl. In other embodiments, R₂, R₃, R₆, and R₇ are each H or cyano. In some embodiments, R₂, R₃, R₆, and R₇ are each H or C₁₋₃ haloalkyl.

In some embodiments, R₂, R₃, and R₇ are each H, nitro, OH, or OCH₃.

In some embodiments, R₂, R₃, and R₇ are each nitro, OH, or OCH₃. In other embodiments, R₂, R₃, and R₇ are each OH or OCH₃.

In some embodiments, R₂ and R₃, together with the atoms to which they are attached, form a 5-7 membered cycloheteroalkyl group. In other embodiments, R₂ and R₃ form methylenedioxy.

In some embodiments, R₂, R₃, and R₇ are each independently H, halo, SCH₃, SO₂CH₃, or CO₂CH₃. In some embodiments, R₂, R₃, and R₇ are each independently H, halo, SCH₃, or CO₂CH₃. In some embodiments, R₂, R₃, and R₇ are each independently H or SO₂CH₃. In some embodiments, R₂, R₃, and R₇ are each independently H or SO₂CH₃. In other embodiments, R₂, R₃, and R₇ are each independently H or halo. In certain embodiments, R₂, R₃, and R₇ are each independently H or fluoro.

In some embodiments, n is 2-4. In other embodiments, n is 3.

In some embodiments, R₉ is NR₁₁R₁₂. In some embodiments, R₁₁ and R₁₂ are each H. In other embodiments, R₁₁ is H and R₁₂ is C₁₋₅ alkyl mono-substituted with OH. In certain embodiments, R₁₁ and R₁₂ together with the nitrogen atom to which they are attached, form a 4-7 membered cycloheteroalkyl or heteroaryl group. In some embodiments, R₁₁ and R₁₂ together with the nitrogen atom to which they are attached, form a morpholine group. In other embodiments, R₁₁ and R₁₂, together with the nitrogen atom to which they are attached, form an imidazole group.

In some embodiments, the DUPA-Indenoisoquinoline conjugate is represented by formula (VII)

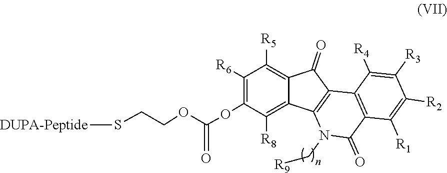

wherein R₁, R₂, R₃, R₄, R₅, R₆, and R₈ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group.

In some embodiments, R₁, R₂, R₃, R₄, R₅, R₆, and R₈ are each independently H, halo, nitro, C₁₋₅ alkyl, O—C₁₋₃ alkyl, S—C₁₋₃ alkyl, SO₂R₁₁, or (CO)OR₁₁; or two adjacent O—C₁₋₃ alkyl groups, together with the atoms to which they are attached, form a 5-7 membered cycloheteroalkyl group.

In some embodiments, R₁, R₄, R₅, and R₈ are each H.

In some embodiments, R₂, R₃, and R₆ are each H, nitro, OH, or OCH₃. In some embodiments, R₂, R₃, and R₆ are each nitro, OH, or OCH₃. In other embodiments, R₂, R₃, and R₆ are each OH or OCH₃. In certain embodiments, R₂, R₃, and R₆ are each OCH₃.

In some embodiments, R₂ and R₃, together with the atoms to which they are attached, form a 5-7 membered cycloheteroalkyl group. In certain embodiments, R₂ and R₃ form methylenedioxy.

In some embodiments, R₂, R₃, and R₆ are each independently H, halo, SCH₃, SO₂CH₃, or CO₂CH₃. In some embodiments, R₂, R₃, and R₆ are each independently H, halo, SCH₃, or CO₂CH₃. In some embodiments, R₂, R₃, and R₆ are each independently H or SO₂CH₃. In certain embodiments, R₂, R₃, and R₆ are each independently H or halo. In other embodiments, R₂, R₃, and R₆ are each independently H or fluoro.

In some embodiments, n is 2-4. In other embodiments, n is 3.

In some embodiments, R₉ is NR₁₁R₁₂. In some embodiments, R₁₁ and R₁₂ are each H. In other embodiments, R₁₁ is H and R₁₂ is C₁₋₅ alkyl mono-substituted with OH. In certain embodiments, R₁₁ and R₁₂ together with the nitrogen atom to which they are attached, form a 4-7 membered cycloheteroalkyl or heteroaryl group. In some embodiments, R₁₁ and R₁₂ together with the nitrogen atom to which they are attached, form a morpholine group. In other embodiments, R₁₁ and R₁₂, together with the nitrogen atom to which they are attached, form an imidazole group.

In some embodiments, the DUPA-Indenoisoquinoline conjugate is represented by formula (IV)

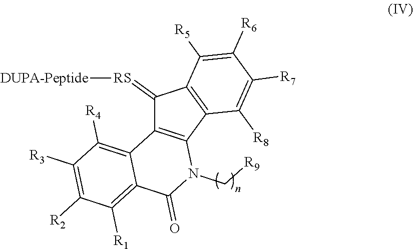

wherein
R₁, R₂, R₃, R₄, R₅, R₆, R₇, and R₈ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group;
R₉ is H, halo, O—C₁₋₅ alkyl, NR₁₁R₁₂, nitro, C₃₋₆ cycloalkyl, or C₃₋₈ cycloheteroalkyl;
R₁₁ and R₁₂ are each independently H or C₁₋₅ alkyl, wherein C₁₋₅ alkyl is optionally mono- or poly-substituted with substituents independently selected from halo, OH, O—C₁₋₃ alkyl, amino, C₁₋₃ alkylamino, and di-C₁₋₃ alkylamino; or R₁₁ and R₁₂, together with the nitrogen atom to which they are attached, form a 4-7 membered cycloheteroalkyl or heteroaryl group; and
n is 0-5.

In some embodiments, RS is a release segment as defined herein.

In some embodiments, n is 2-4. In other embodiments, n is 3. In other embodiments, n is 1.

In some embodiments, the DUPA-Indenoisoquinoline conjugate is represented by formula (VIII)

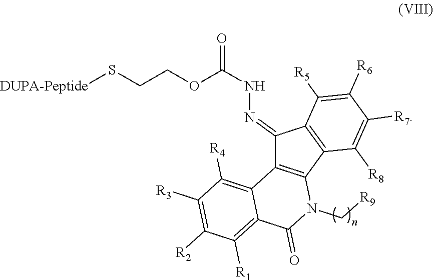

In some embodiments, R₁, R₂, R₃, R₄, R₅, R₆, R₇, and R₈ are each independently H, halo, nitro, C₁₋₅ alkyl, O—C₁₋₃ alkyl, cyano, C₁₋₃ haloalkyl, S—C₁₋₃ alkyl, SO₂R₁₁, or (CO)OR₁₁; or two adjacent O—C₁₋₃ alkyl groups, together with the atoms to which they are attached, form a 5-7 membered cycloheteroalkyl group.

In some embodiments, R₁, R₄, R₅, and R₈ are each H.

In some embodiments, R₂, R₃, R₆, and R₇ are each H, nitro, OH, OCH₃, cyano, C₁₋₃ haloalkyl. In some embodiments, R₂, R₃, R₆, and R₇ are each H, cyano, or C₁₋₃ haloalkyl. In other embodiments, R₂, R₃, R₆, and R₇ are each H or cyano. In some embodiments, R₂, R₃, R₆, and R₇ are each H or C₁₋₃ haloalkyl.

In some embodiments, R₂, R₃, R₆, and R₇ are each H, nitro, OH, or OCH₃. In some embodiments, R₂, R₃, R₆, and R₇ are each nitro, OH, or OCH₃. In other embodiments, R₂, R₃, R₆, and R₇ are each independently H, halo, SCH₃, SO₂CH₃, or CO₂CH₃. In some embodiments, R₂, R₃, R₆, and R₇ are each independently H, halo, SCH₃, or CO₂CH₃. In some embodiments, R₂, R₃, R₆, and R₇ are each independently H or SO₂CH₃. In certain embodiments, R₂, R₃, R₆, and R₇ are each independently H or halo. In some embodiments, R₂, R₃, R₆, and R₇ are each independently H or fluoro

In some embodiments, R₂, R₃, R₆, and R₇ are each OH or OCH₃.

In some embodiments, R₂ and R₃, together with the atoms to which they are attached, form a 5-7 membered cycloheteroalkyl group. In certain embodiments, R₂ and R₃ form methylenedioxy.

In other embodiments, R₆ and R₇, together with the atoms to which they are attached, form a 5-7 membered cycloheteroalkyl group. In certain embodiments, R₆, and R₇ form methylenedioxy.

In some embodiments, n is 2-4. In other embodiments, n is 3.

In some embodiments, R₉ is NR₁₁R₁₂. In some embodiments, R₁₁ and R₁₂ are each H. In other embodiments, R₁₁ is H and R₁₂ is C₁₋₅ alkyl monos-ubstituted with OH. In some embodiments, R₁₁ and R₁₂ together with the nitrogen atom to which they are attached, form a 4-7 membered cycloheteroalkyl or heteroaryl group. In certain embodiments, R₁₁ and R₁₂ together with the nitrogen atom to which they are attached, form a morpholine group. In some embodiments, R₁₁ and R₁₂, together with the nitrogen atom to which they are attached, form an imidazole group.

In some embodiments, the DUPA-Indenoisoquinoline conjugate is represented by formula (IX)

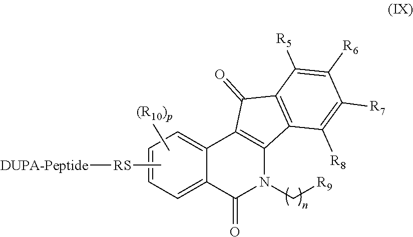

wherein
R₅, R₆, R₇, R₈, and R₁₀ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group;
R₉ is H, halo, O—C₁₋₅ alkyl, NR₁₁R₁₂, nitro, C₃₋₆ cycloalkyl, or C₃₋₈ cycloheteroalkyl;
R₁₁ and R₁₂ are each independently H or C₁₋₅ alkyl, wherein C₁₋₅ alkyl is optionally mono- or poly-substituted with substituents independently selected from halo, OH, O—C₁₋₃ alkyl, amino, C₁₋₃ alkylamino, and di-C₁₋₃ alkylamino; or R₁₁ and R₁₂, together with the nitrogen atom to which they are attached, form a 4-7 membered cycloheteroalkyl or heteroaryl;
n is 0-5; and
p is 3.

In some embodiments, n is 2-4. In other embodiments, n is 3. In other embodiments, n is 1.

In some embodiments, the DUPA-Indenoisoquinoline conjugate is represented by formulas (X)

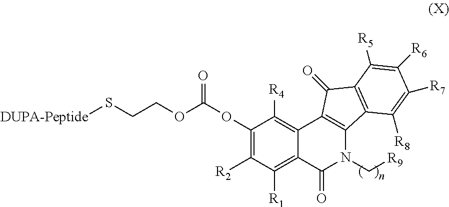

wherein R₁, R₂, and R₄ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group.

In some embodiments, R₁, R₂, R₄, R₅, R₆, R₇, and R₈ are each independently H, halo, nitro, C₁₋₅ alkyl, O—C₁₋₃ alkyl, S—C₁₋₃ alkyl, SO₂R₁₁, or (CO)OR₁₁; or two adjacent O—C₁₋₃ alkyl groups, together with the atoms to which they are attached, form a 5-7 membered cycloheteroalkyl group.

In some embodiments, R₁, R₄, R₅, and R₈ are each H.

In some embodiments, R₂, R₆, and R₇ are each H, nitro, OH, or OCH₃. In other embodiments, R₂, R₆, and R₇ are OH or OCH₃.

In some embodiments, R₆ and R₇, together with the atoms to which they are attached, form a 5-7 membered cycloheteroalkyl group. In other embodiments, R₆ and R₇ form methylenedioxy.

In some embodiments, R₂, R₆, and R₇ are each independently H, halo, SCH₃, SO₂CH₃, or CO₂CH₃. In some embodiments, R₂, R₆, and R₇ are each independently H, halo, SCH₃, or CO₂CH₃. In some embodiments, R₂, R₆, and R₇ are each independently H or SO₂CH₃. In some embodiments, R₂, R₆, and R₇ are each independently H or halo. In certain embodiments, R₂, R₆, and R₇ are each independently H or fluoro.

In some embodiments, n is 2-4. In certain embodiments, n is 3.

In some embodiments, R₉ is NR₁₁R₁₂. In some embodiments, R₁₁ and R₁₂ together with the nitrogen atom to which they are attached, form a 4-7 membered cycloheteroalkyl or heteroaryl group. In some embodiments, R₁₁ and R₁₂ together with the nitrogen atom to which they are attached, form a morpholine group. In other embodiments, R₁₁ and R₁₂, together with the nitrogen atom to which they are attached, form an imidazole group.

In some embodiments, the DUPA-Indenoisoquinoline conjugate is represented by formulas (XI)

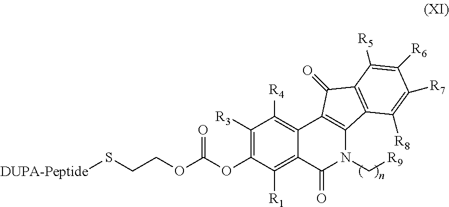

wherein R₁, R₃, and R₄ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group.

In some embodiments, R₁, R₃, R₄, R₅, R₆, R₇, and R₈ are each independently H, halo, nitro, C₁₋₅ alkyl, O—C₁₋₃ alkyl, S—C₁₋₃ alkyl, SO₂R₁₁, or (CO)OR₁₁; or two adjacent O—C₁₋₃ alkyl groups, together with the atoms to which they are attached, form a 5-7 membered cycloheteroalkyl group.

In some embodiments, R₁, R₄, R₅, and R₈ are each H.

In some embodiments, R₃, R₆, and R₇ are each H, nitro, OH, or OCH₃. In certain embodiments, R₃, R₆, and R₇ are OH or OCH₃.

In some embodiments, R₆ and R₇, together with the atoms to which they are attached, form a 5-7 membered cycloheteroalkyl group. In some embodiments, R₆ and R₇ form methylenedioxy.

In other embodiments, R₃, R₆, and R₇ are each independently H, halo, SCH₃, SO₂CH₃, or CO₂CH₃. In some embodiments, R₃, R₆, and R₇ are each independently H, halo, SCH₃, or CO₂CH₃. In some embodiments, R₃, R₆, and R₇ are each independently H or SO₂CH₃. In certain embodiments, R₃, R₆, and R₇ are each independently H or halo. In other embodiments, R₃, R₆, and R₇ are each independently H or fluoro.

In some embodiments, n is 2-4. In certain embodiments, n is 3.

In some embodiments, R₉ is NR₁₁R₁₂. In some embodiments, R₁₁ and R₁₂ together with the nitrogen atom to which they are attached, form a 4-7 membered cycloheteroalkyl or heteroaryl group. In certain embodiments, R₁₁ and R₁₂ together with the nitrogen atom to which they are attached, form a morpholine group. In other embodiments, R₁₁ and R₁₂, together with the nitrogen atom to which they are attached, form an imidazole group.

In some embodiments, the DUPA-Indenoisoquinoline conjugate is represented by formula (XII)

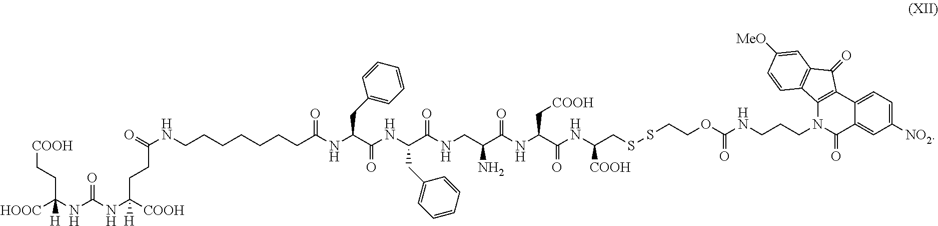 [SMILES: COc1ccc2c(c1)C(=O)c1c-2n(CCCNC(=O)OCCSSC[C@H](NC(=O)[C@H](CC(=O)O)NC(=O)[C@@H](N)CNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)C(=O)O)c(=O)c2cc([N+](=O)[O-])ccc12]

In some embodiments, the DUPA-Indenoisoquinoline conjugate is represented by formula (XIII)

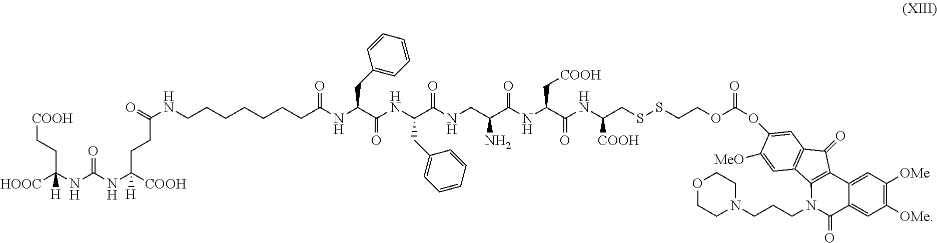 [SMILES: COc1cc2c3c(n(CCCN4CCOCC4)c(=O)c2cc1OC)-c1cc(OC)c(OC(=O)OCCSSC[C@H](NC(=O)[C@H](CC(=O)O)NC(=O)[C@@H](N)CNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)C(=O)O)cc1C3=O]

In some embodiments, the DUPA-Indenoisoquinoline conjugate is represented by formula (XIV)

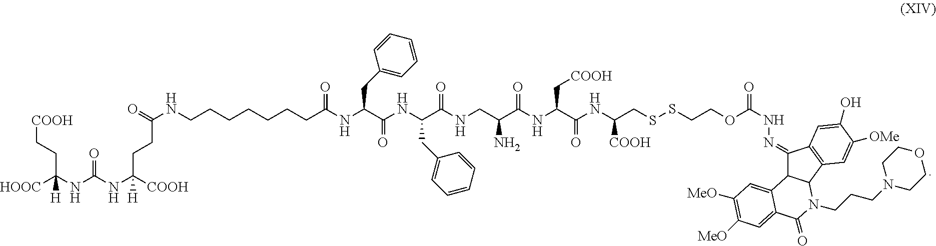 [SMILES: COc1cc2c(cc1O)/C(=N\NC(=O)OCCSSC[C@H](NC(=O)[C@H](CC(=O)O)NC(=O)[C@@H](N)CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)O)C1c3cc(OC)c(OC)cc3C(=O)N(CCCN3CCOCC3)C21]

In some embodiments, the DUPA-Indenoisoquinoline conjugate is represented by formula (XV)

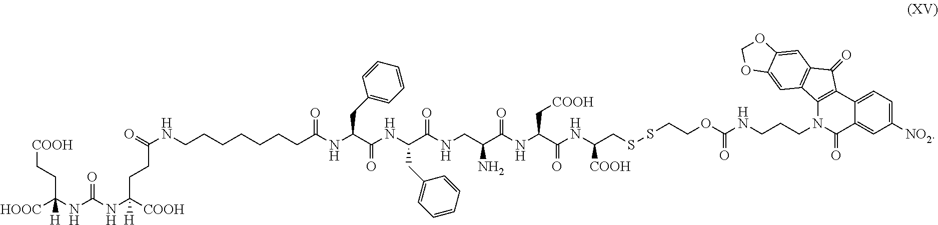 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CSSCCOC(=O)NCCCn1c2c(c3ccc([N+](=O)[O-])cc3c1=O)C(=O)c1cc3c(cc1-2)OCO3)C(=O)O]

In some embodiments, the DUPA-Indenoisoquinoline conjugate is represented by formula (XVI)

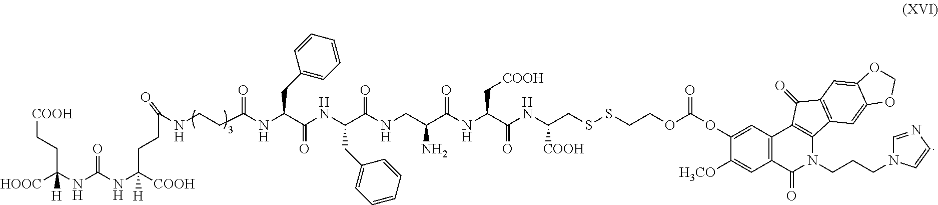

In some embodiments, the DUPA-Indenoisoquinoline conjugate is represented by formula (XVII)

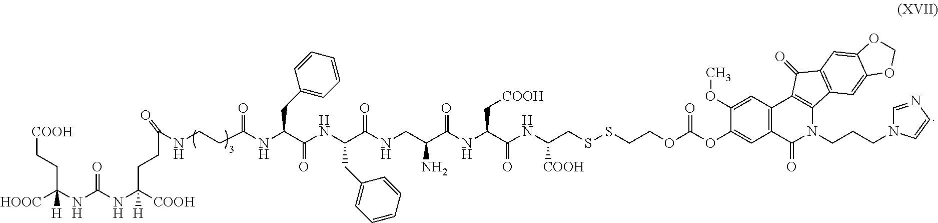

In some embodiments, the DUPA-Indenoisoquinoline conjugate is represented by formula (XVIII)

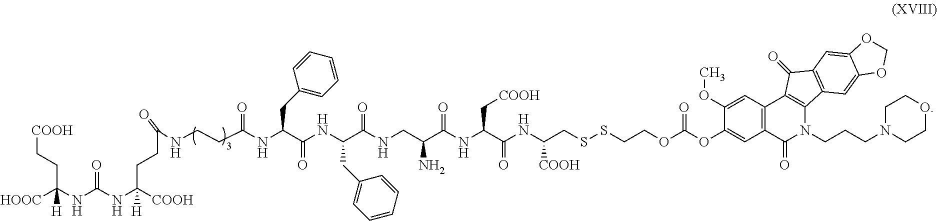

In some embodiments, the DUPA-Indenoisoquinoline conjugate is represented by formula (XIX)

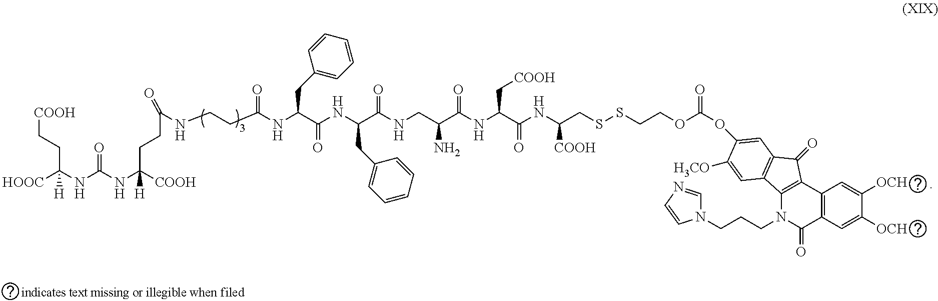

In some embodiments, the DUPA-Indenoisoquinoline conjugate is represented by formula (XX)

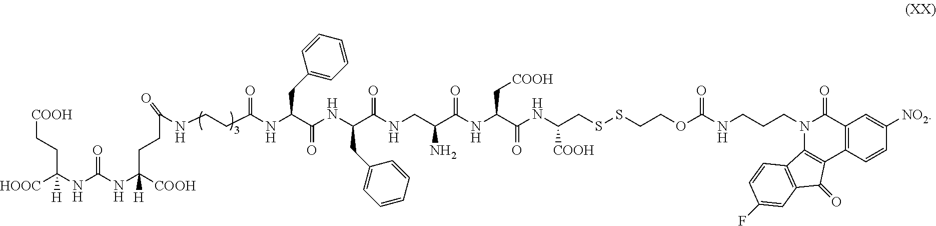

In some embodiments, the bond between RS and Indenoisoquinoline is cleaved under a suitable condition. In some embodiments, the suitable condition is within cells. In some embodiments, the cells are cancer cells. In certain embodiments, the cells are prostate cancer cells.

In another aspect, the present invention provides a process of preparing a DUPA-Indenoisoquinoline conjugate represented by formula (IB):

DUPA-Linker-RS-Indenoisoquinoline (IB)

wherein
DUPA is a modified or unmodified 2-[3-(1,3-dicarboxypropyl)-ureido]pentanedioic acid;
Linker is a bond, a substituted or unsubstituted alkyl, a peptide, or a peptidoglycan;
Indenoisoquinoline is a substituted or unsubstituted indenoisoquinoline; and
RS is a release segment capable of releasing Indenoisoquinoline within the desired cells, wherein said release segment is a carbonate segment, a carbamate segment, or an acylhydrazone segment.
   the process comprising
(d) reacting a DUPA with a peptide to prepare a DUPA-peptide reagent;
(e) reacting an RS reagent with an indenoisoquinoline to prepare an RS-indenoisoquinoline compound; and
(f) reacting the DUPA-peptide reagent of step (a) with the RS-indenoisoquinoline compound of step (b) to prepare said DUPA-Indenoisoquinoline conjugate.

In some embodiments, the RS reagent is represented by formula (XXI)

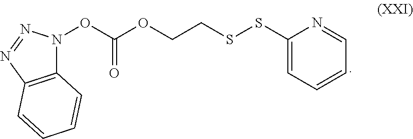 [SMILES: O=C(OCCSSc1ccccn1)On1nnc2ccccc21]

In some embodiments, the DUPA-peptide reagent is represented by formula (XXII)

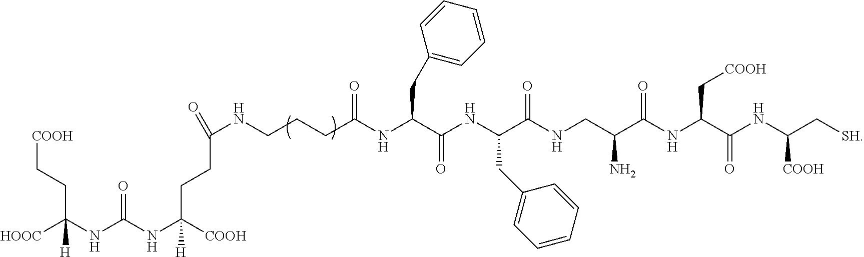

The general syntheses of indenoisoquinoline Top1 inhibitors 1-19, 87, and 88 (Scheme 1) and their DUPA conjugates are exemplified in Schemes 1 and 2:

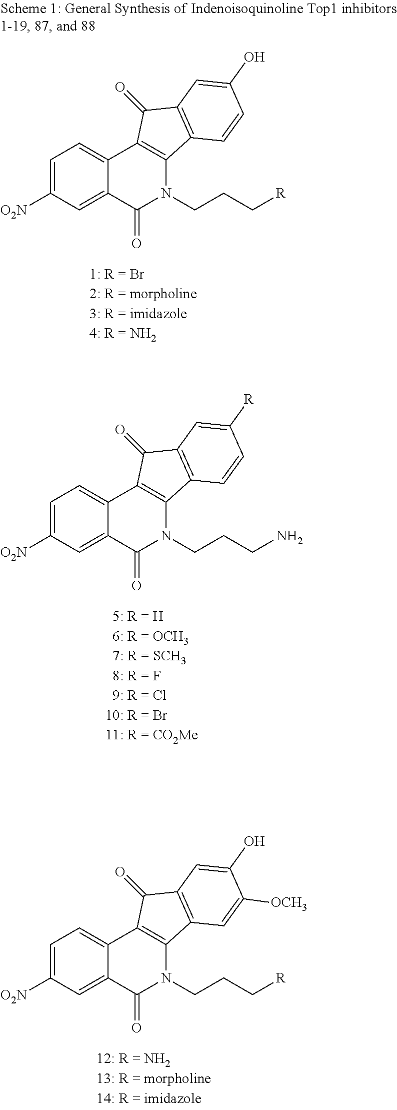

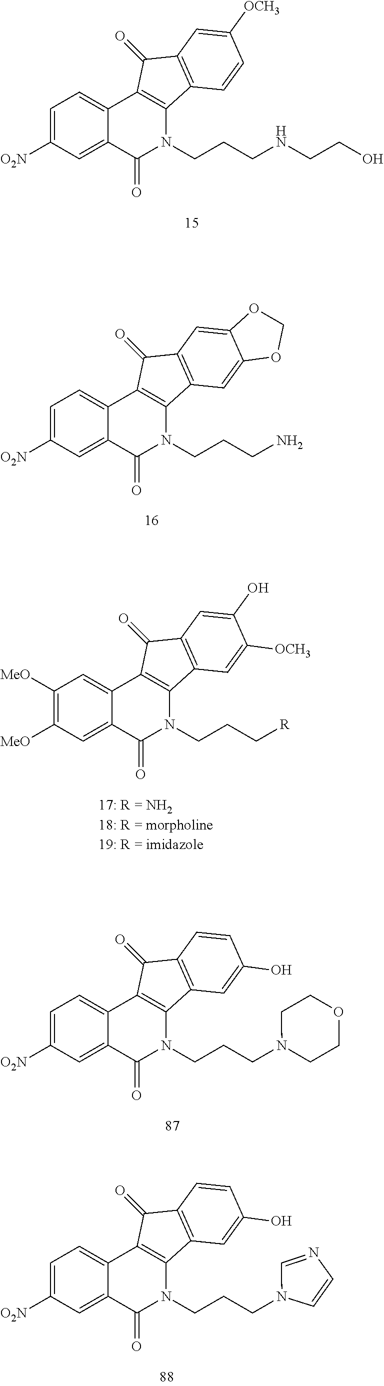

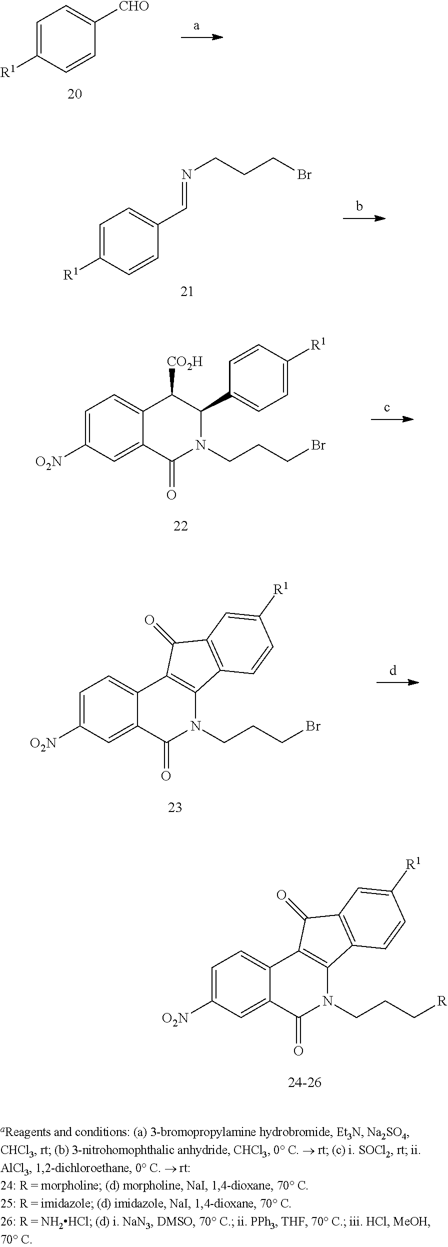

In Scheme 1, the reaction of benzaldehyde 20 (sensitive functional groups in 20 were protected when necessary) with 3-bromopropylamine provided Schiff base 21, which upon condensation with 3-nitrohomophthalic anhydride yielded cis acid 22 in good yield and purity; treatment of acid 22 with SOCl₂, followed by AlCl₃, afforded indenoisoquinoline bromide 23; displacement of the bromine atom in 23 with an appropriate base (morpholine, imidazole, or azide, followed by Staudinger reduction and acidic hydrolysis) provided the desired corresponding amines 24-26.

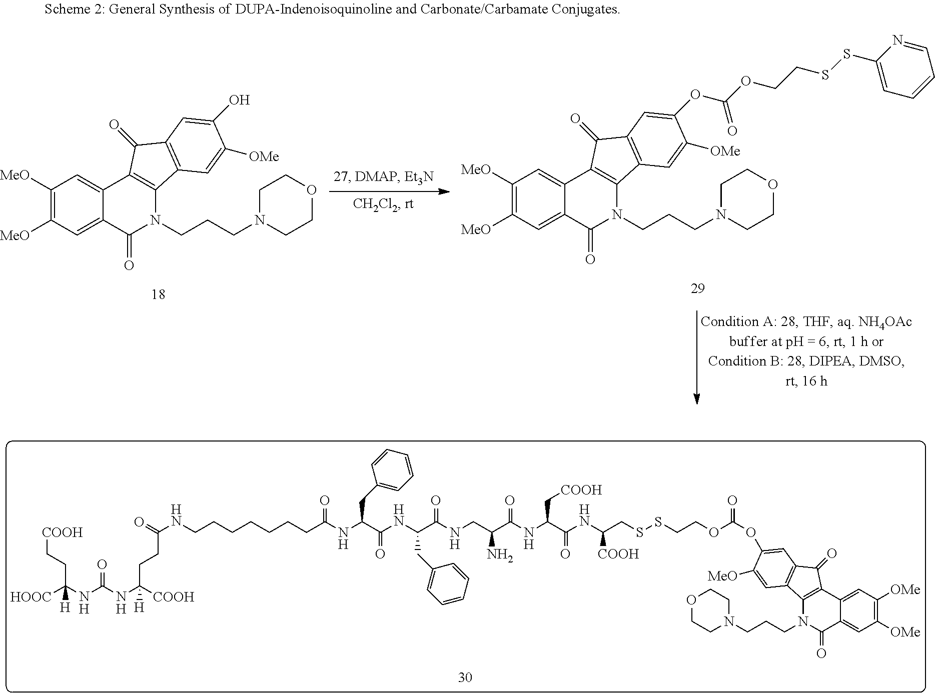 [SMILES: COc1cc2c(cc1O)C(=O)c1c-2n(CCCN2CCOCC2)c(=O)c2cc(OC)c(OC)cc12 ; COc1cc2c3c(n(CCCN4CCOCC4)c(=O)c2cc1OC)-c1cc(OC)c(OC(=O)OCCSSc2ccccn2)cc1C3=O ; COc1cc2c3c(n(CCCN4CCOCC4)c(=O)c2cc1OC)-c1cc(OC)c(OC(=O)OCCSSC[C@H](NC(=O)[C@H](CC(=O)O)NC(=O)[C@@H](N)CNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)C(=O)O)cc1C3=O]

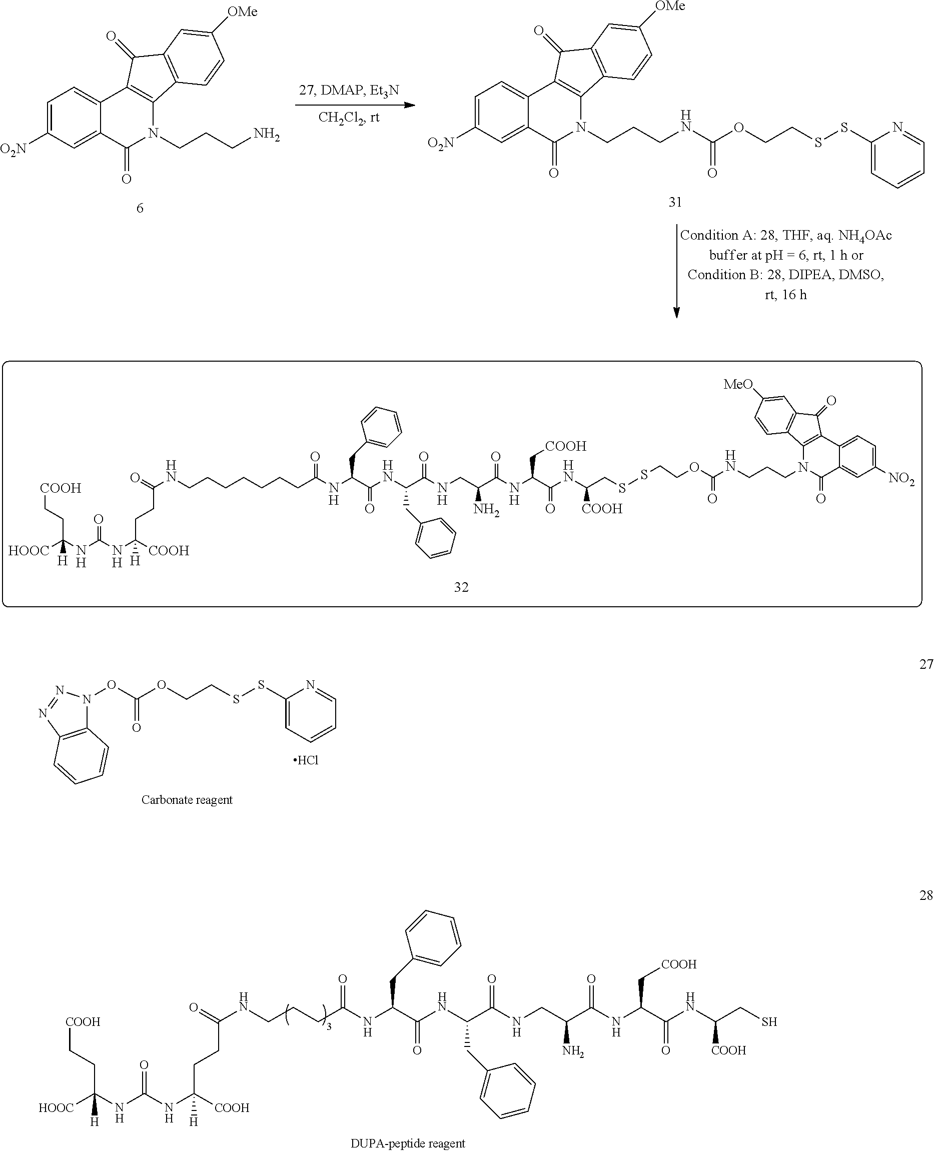

In Scheme 2, reaction of carbonate reagent 27 with phenol 18 or amine 6 yielded the carbonate 29 or carbamate 31, respectively; treatment of 29 or 31 with DUPA-peptide reagent 28 in either mildly acidic aqueous medium (condition A) or in DMSO and base DIPEA (condition B) afforded the corresponding DUPA-drug conjugates 30 and 32, respectively. In some parts of the application, conjugate 30 is conjugate 86, and conjugate 32 is conjugate 84.

DUPA-Indenoisoquinoline conjugates (XVI)-(XX) can be prepared by the methods described in Scheme 2.

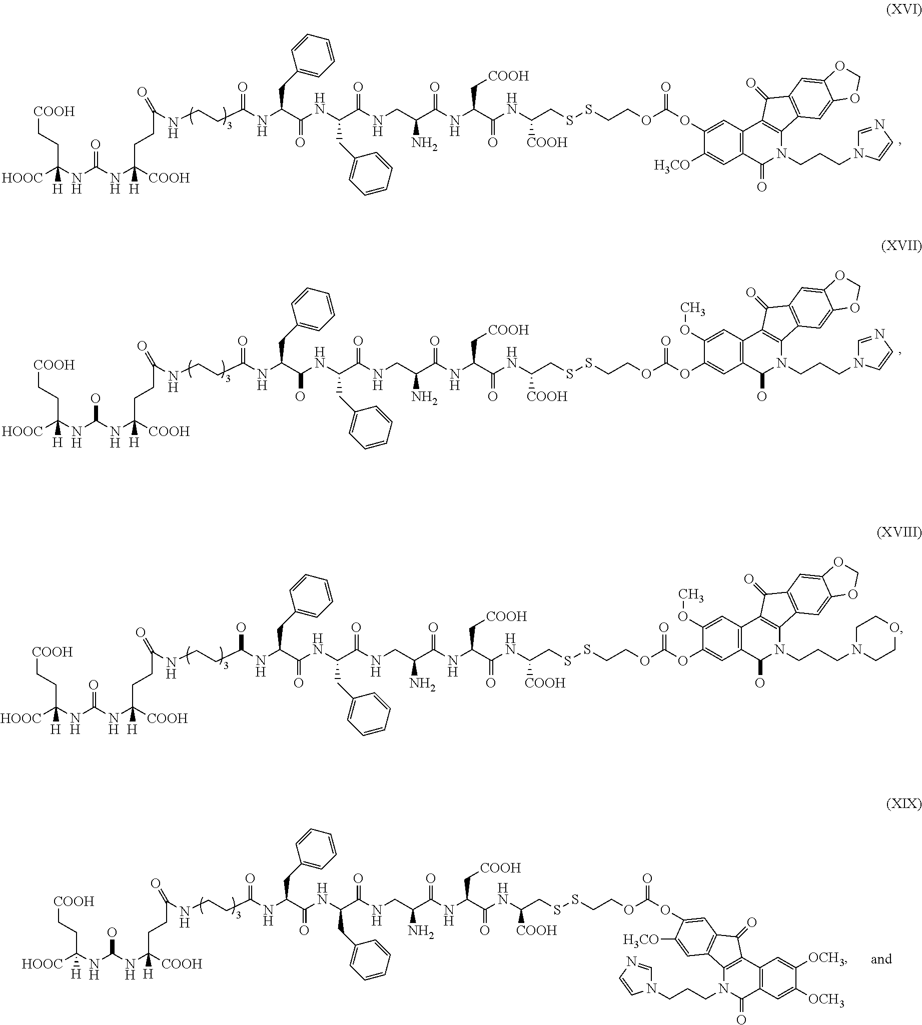

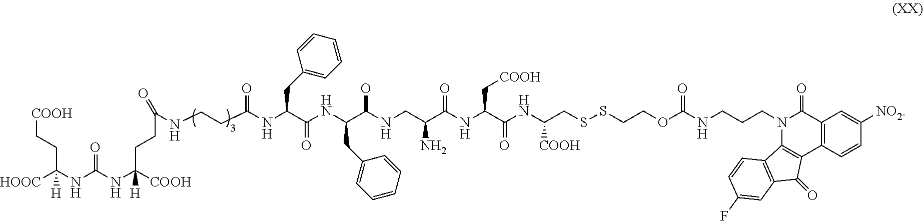

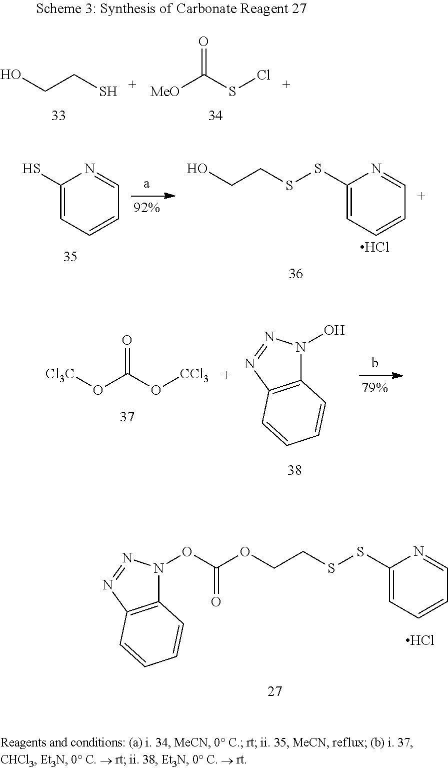

In Scheme 3, treatment of 2-mercaptoethanol (33) with sulfenyl chloride 34, followed by an addition of pyridine 35 in CH₃CN at reflux provided alcohol 36 as an HCl salt. Reaction of 36 with triphosgene (37) afforded a carbonate intermediate of phosgene, which upon ester exchange reaction with triazole 38 yielded the desired carbonate reagent 27 in excellent yield (79% overall) and purity.

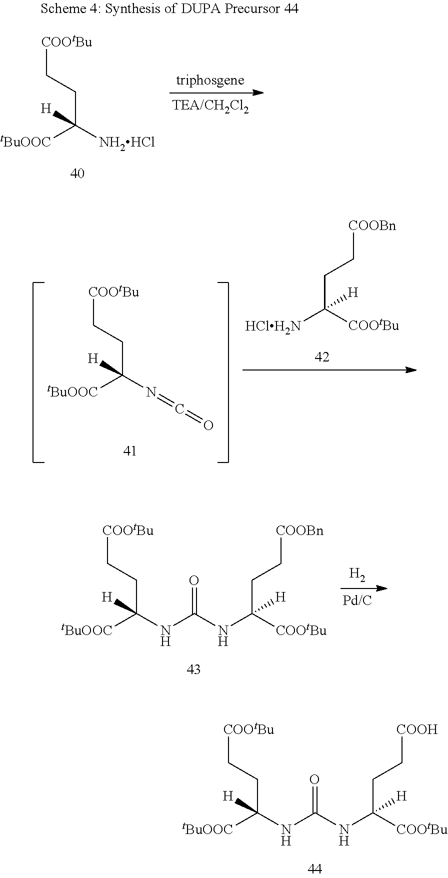

In Scheme 4, α,γ-dibenzylglutamate 40 and triphosgene were treated with triethylamine (TEA) at −78° C. in inert atmosphere for 2 h to provide isocyanate 41 (or α,γ-dibenzylglutamate 40 was treated with triphosgene in the presence of triethylamine (TEA) at 0° C. in inert atmosphere for 2 h to provide isocyanate 41). Addition of glutamate 42 with overnight stirring yielded urea 43 after workup and column chromatography. Atmospheric hydrogenation of 42 in EtOAc overnight (or for two days) and in the presence of Pd—C afforded the pure DUPA precursor 44 in 100% yield.

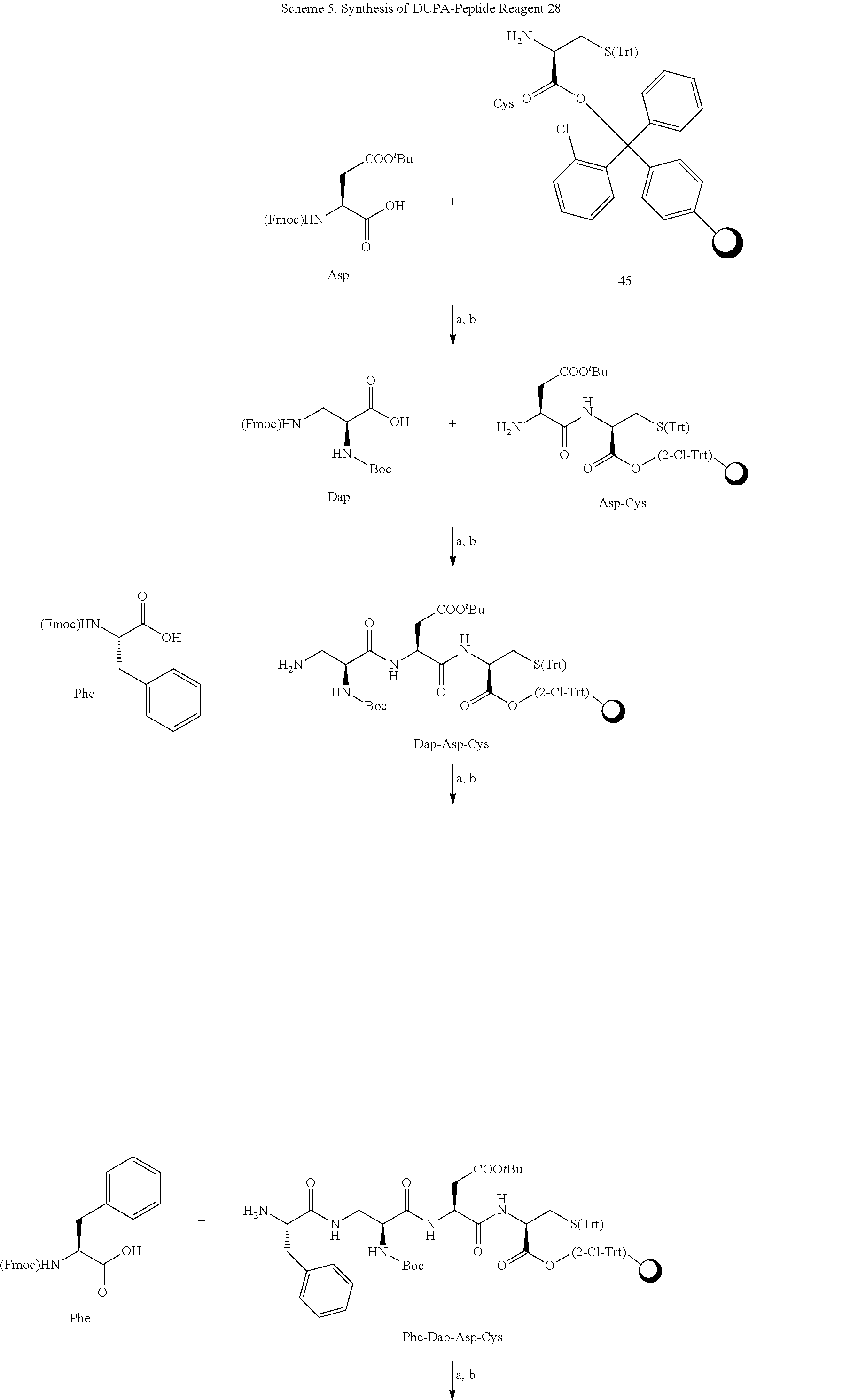

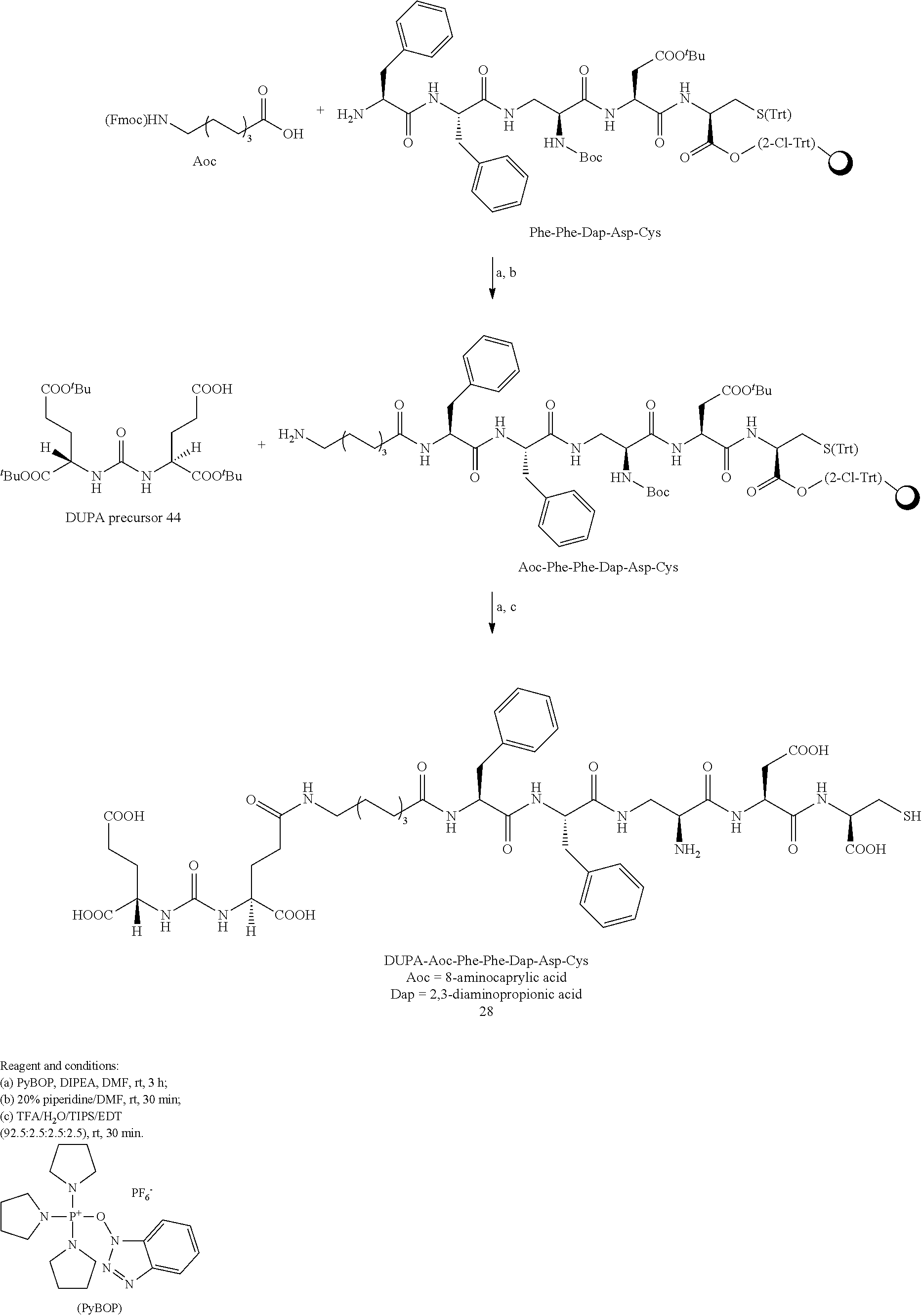

In Scheme 5, Fmoc-solid phase peptide synthesis was used to prepare DUPA-peptide reagent 28, starting with Fmoc-free H-Cys(Trt)-(2-ClTrt) resin (45) in place of the reported Fmoc-Cys(Trt)-(4-MeOTrt) resin, because the reagent 45 could not only suppress the racemization of L-Cys to D-Cys, but also save the time that the first Fmoc-cleavage would have taken if the Fmoc-Cys(Trt)-(4-MeOTrt) resin was used.

In order to be effective, anticancer drugs (e.g., indenoisoquinolines) attached to the DUPA moiety must be connected in a way that confers stability in solution until it gets inside the prostate cancer cells, and the linkage must support a release mechanism, e.g., a facile drug-release mechanism, that will then free the drug. In the present invention, the release mechanism involves disulfide reduction of the DUPA-drug conjugate 30 by glutathione within the reducing environment of endosomes to yield the intermediate 46 (Scheme 6) (Leamon, et al. Cancer Res. 2008, 68, 9839-9844).

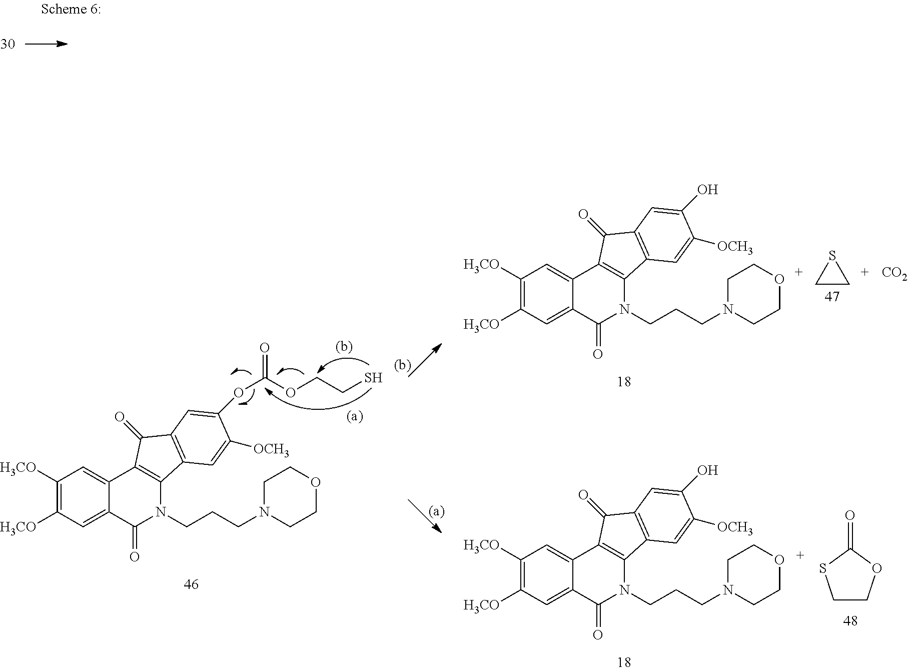

In Scheme 6, the sulfhydryl group in intermediate 46 was designed to undergo intramolecular nucleophilic attack either via path (a) to liberate the parent drug 18 and 1,3-oxathiolan-2-one (48), or path (b) to give the free drug 18, thiirane (47), and carbon dioxide (Kularatne, et al., J. Med. Chem. 2010, 53, 7767-7777).

The antitumor activity of DUPA-Indenoisoquinoline conjugates can be tested in an animal model in the presence of a high-affinity PSMA ligand, 2-(phosphonomethyl)pentanedioic acid (PMPA) (Kᵢ=0.275 nM) (Jackson, et al., J. Med. Chem. 1996, 39, 619-622), which acted as a competitor and was used in 100-fold excess of the DUPA-Indenoisoquinoline conjugate. If the activity is completely blocked (i.e. the tumor volume in xenograft model is similar to that of the control group), the result would support that the activity and uptake of DUPA-Indenoisoquinoline conjugates must be PSMA-mediated, implying that the free drug (e.g., indenoisoquinoline) must be liberated inside the cell instead of outside the cell. The toxicity of the conjugate should be reduced since it will not be absorbed by cells that lack PSMA.

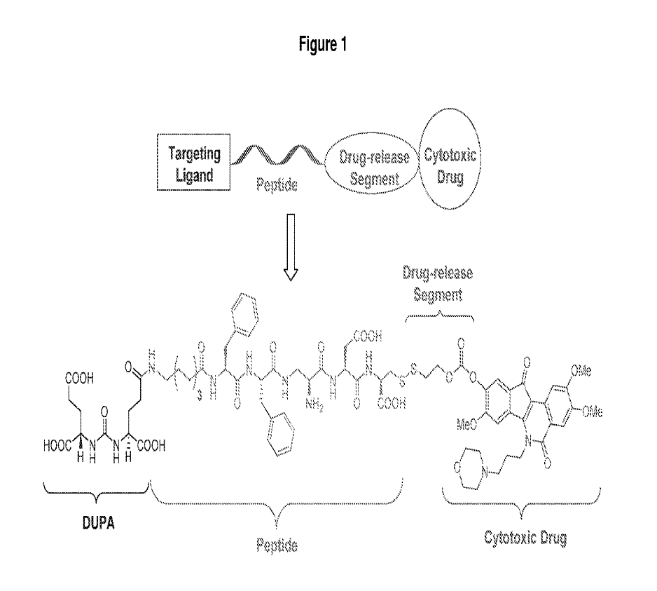

The DUPA-Indenoisoquinoline conjugates of the present invention include four components of the conjugate: targeting ligand (e.g., DUPA), linker (e.g., peptide), drug-release segment, and cytotoxic drug (e.g., indenoisoquinolines). FIG. 1 shows a general schematic representation of a ligand-drug conjugate used in the concept of ligand-targeted therapeutics. The tumor-targeting ligand (DUPA) is connected to the cytotoxic indenoisoquinoline Top1 inhibitor by a peptide linker and a carbonate drug-release segment that will facilitate the release of the free drug inside the target cell. These four components of the conjugate could be modified for various purposes, including binding optimization, potency enhancement, and cancer specificity/selectivity.

In the drug conjugates of the present invention, the drug can be any cytotoxic drugs including those known by one of skill in the art and medical practitioners, for example, topoisomerase I inhibitors. In some embodiments, the drug conjugates is the DUPA-Indenoisoquinoline conjugates. In some embodiments, the Indenoisoquinoline of the DUPA-Indenoisoquinoline conjugates is an indenoisoquinoline compound as described herein. The DUPA-Indenoisoquinoline conjugates can be used for the treatment of cancers, for example, ovarian cancer, lung cancer, breast cancer, or prostate cancer. In some embodiments, the DUPA-Indenoisoquinoline conjugates can be used for the treatment of prostate cancer.

In the drug conjugates of the present invention, e.g., DUPA-Indenoisoquinoline conjugates, DUPA shows high affinity for PSMA (also called folate hydrolase I or glutamate carboxypeptidase II), which is a type II membrane glycoprotein (Kᵢ=8 nM) (Kozikowski, et al., J. Med. Chem. 2004, 47, 1729-1738). Upon binding to DUPA, PSMA undergoes endocytosis, unloads the ligand, and then recycles rapidly to the cell surface. In some embodiments, DUPA can be modified. For example, DUPA can be substituted with an alkyl group, a hydroxy group, an alkoxy group, a thio group, a phosphate or thiophosphate group, a cyano group, or other substituents known in the art. In other embodiments, DUPA is not modified.

In the drug conjugates of the present invention, e.g., DUPA-Indenoisoquinoline conjugates, the linker can be any spacer known in the art to be able to link DUPA and drug (e.g., Indenoisoquinoline). In some embodiments, the linker is a bond. In some embodiments, the linker is an alkyl chain, which can be substituted or unsubstituted with one or more heteroatoms. In other embodiments, the linker is a peptide or a peptidoglycan. In some embodiments, the length and chemical composition of the linker (e.g., peptide) can be modified so that it would help enhance the binding affinity of the DUPA ligand to PSMA. The linker can also be modified to be more hydrophilic or hydrophobic to balance the hydrophobicity or hydrophilicity of the drugs or conjugates of the invention.

The release segment (e.g., RS) of the conjugates of the invention is capable of releasing the drug in the conjugate, e.g., indenoisoquinoline, within the desired cells. In some embodiments, the release segment is a carbonate segment, a carbamate segment, or an acylhydrazone segment. In certain embodiments, the release segment is a carbonate segment.

In the conjugates of the present invention, e.g., DUPA-Indenoisoquinoline, the indenoisoquinoline possesses a reactive hydroxyl or amine group that is suitable for further conjugation.

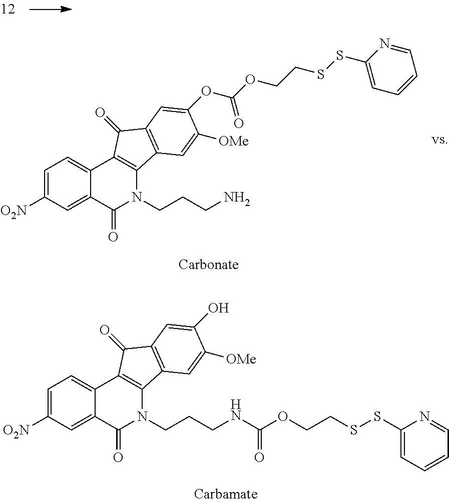 [SMILES: COc1cc2c(cc1OC(=O)OCCSSc1ccccn1)C(=O)c1c-2n(CCCN)c(=O)c2cc([N+](=O)[O-])ccc12 ; COc1cc2c(cc1O)C(=O)c1c-2n(CCCNC(=O)OCCSSc2ccccn2)c(=O)c2cc([N+](=O)[O-])ccc12]

The stability of the present carbonate or carbamate linkage is an important factor that determines the cytotoxicity of the free drug and the feasibility of the current methodology, because it must be both sufficiently stable in blood plasma to reach prostate cancer cells, and sufficiently labile to release the free drug once the conjugate is inside the cell. Such factor must be monitored to determine the most suitable linkage for the indenoisoquinoline drugs. In some embodiments, indenoisoquinoline compounds 4, 12, 15, and 17 are desired candidates for the conjugates of the invention. In other embodiments, indenoisoquinoline compounds 6, 16, and 18 are desired candidates for the conjugates of the invention.

In some embodiments, in addition to the attachments shown anywhere herein, the release segment (e.g., RS) in the DUPA-Indenoisoquinoline conjugates can be attached to the conjugates as exemplified in Scheme 7. Thus, the conjugates can undergo an imine hydrolysis: reaction of carbonate reagent 27 with hydrazine provided the hydrazide intermediate 49, which upon treatment with 18 would afford the acylhydrazone 50; similar treatment of disulfide 50 with the DUPA-peptide 28 in DMSO and DIPEA (condition B) would yield the desired product 51. Upon internalization in form of endosome, the free drug (e.g., indenoisoquinolines) would be released intracellularly from its conjugate via the acid-catalyzed acylhydrozone hydrolysis of the conjugate at endosomal pH, a release mechanism that has been employed successfully in the case of doxorubicin (see Zhou, et al., Biomacromolecules 2011, 12, 1460-1467; Yoo, et al., J. Controlled Release 2002, 82, 17-27; Lee, et al., Proc. Natl. Acad. Sci. U.S. Pat. No. 2,006,103, 16649-16654; Bae, et al., Angew. Chem. Int. Ed. 2003, 42, 4640-4643; and Hu, et al., Biomacromolecules 2010, 11, 2094-2102).

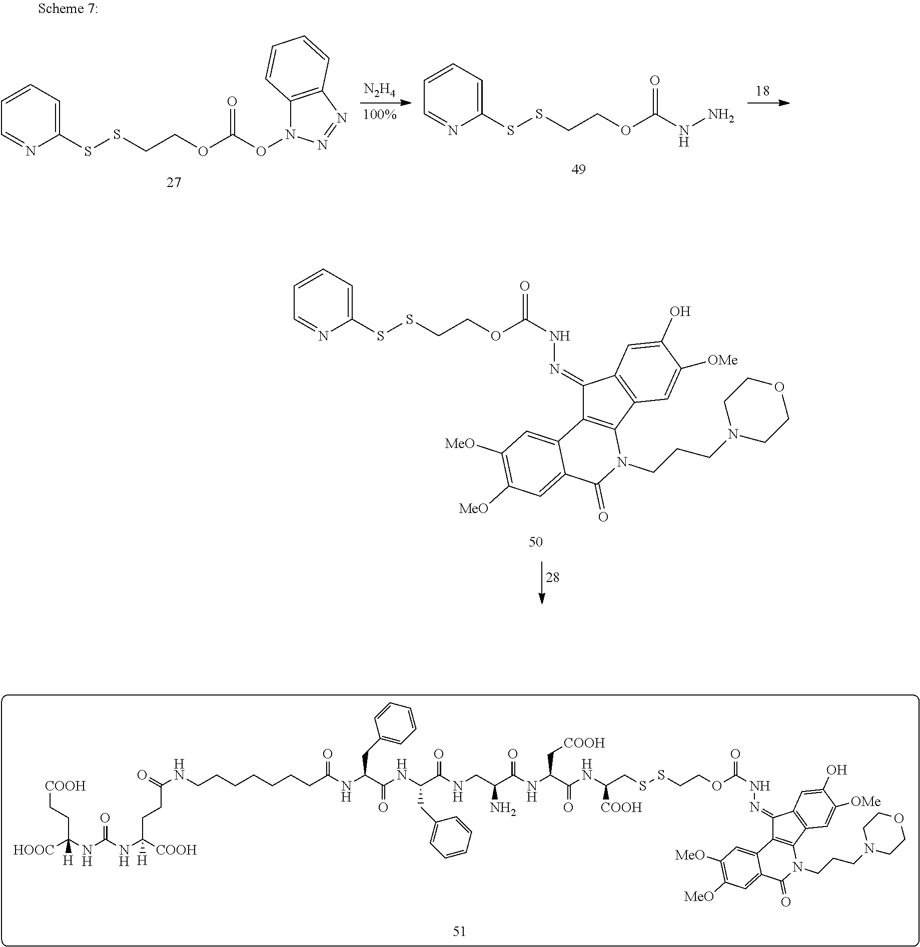 [SMILES: O=C(OCCSSc1ccccn1)On1nnc2ccccc21 ; "N2H4" ; NNC(=O)OCCSSc1ccccn1 ; "18" ; COc1cc2c(cc1O)/C(=N\NC(=O)OCCSSc1ccccn1)c1c-2n(CCCN2CCOCC2)c(=O)c2cc(OC)c(OC)cc12 ; COc1cc2c(cc1O)/C(=N\NC(=O)OCCSSC[C@H](NC(=O)[C@H](CC(=O)O)NC(=O)[C@@H](N)CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)O)c1c-2n(CCCN2CCOCC2)c(=O)c2cc(OC)c(OC)cc12]

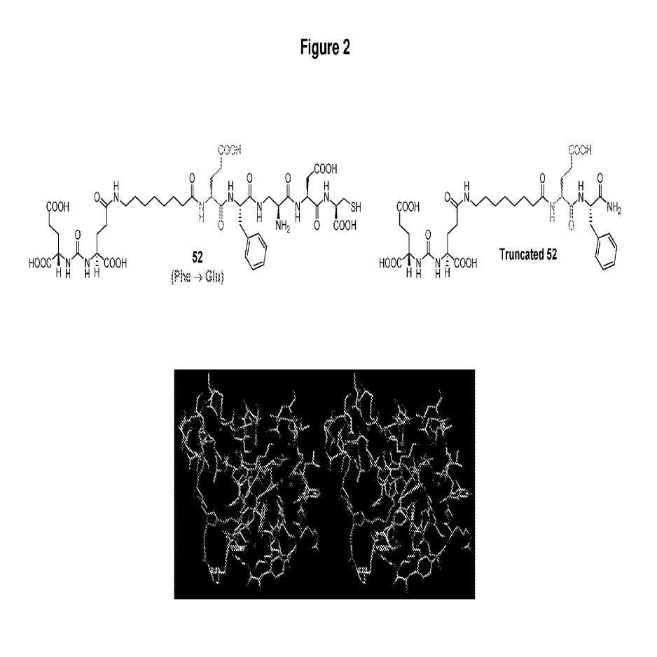

The peptide of the DUPA-Indenoisoquinoline conjugate of the invention served both as a spacer between the DUPA ligand and the cytotoxic drugs in order to ensure the binding of PSMA and its ligand, and as a means to improve the water solubility of indenoisoquinolines. All DUPA-drug conjugates (e.g., 30 and 32 or 84 and 86) dissolve completely and easily in water at room temperature. In some embodiments, the length and chemical composition of the peptide linker can be modified so that it would help enhance the binding affinity of the DUPA ligand to PSMA. As an example of a modification, the structure of peptide linker 28 has been changed by substitution of the internal phenylalanine with a glutamic acid residue, and the truncated form of the resulting compound 52 was docked and energy-minimized on the PSMA target as shown in FIG. 2 (note that the truncated form was used for simplicity and quick representation of the rationales in this approach). The new Glu residue would improve the aqueous solubility of the linker, and its theoretical model revealed a potential salt bridge (2.23 Å) with the terminal side chain ammonium cation of Lys207 (bottom left) in the PSMA crystal structure.

### DEFINITIONS

At various places in the present specification, substituents of compounds of the invention are disclosed in groups or in ranges. It is specifically intended that the invention include each and every individual subcombination of the members of such groups and ranges. For example, the term “C₁₋₅ alkyl” is specifically intended to individually disclose methyl, ethyl, C₃ alkyl, C₄ alkyl, and C₅ alkyl.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

In some embodiments, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms.

In some embodiments, “haloalkyl” refers to an alkyl group having one or more halogen substituents. Example haloalkyl groups include CF₃, C₂F₅, CHF₂, CCl₃, CHCl₂, C₂Cl₅, and the like.

In some embodiments, “aryl” refers to monocyclic or polycyclic (e.g., having 2, 3 or 4 fused rings) aromatic hydrocarbons such as, for example, phenyl, naphthyl, anthracenyl, phenanthrenyl, and the like. In some embodiments, an aryl group has from 6 to about 20 carbon atoms.

In some embodiments, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of cyclopentane, cyclopentene, cyclohexane, and the like. A cycloalkyl group having one or more fused aromatic rings can be attached through either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like.

In some embodiments, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

In some embodiments, “cycloheteroalkyl” or “heterocycloalkyl” refers to a non-aromatic heterocycle where one or more of the ring-forming atoms are a heteroatom such as an O, N, or S atom. Cycloheteroalkyl or heterocycloalkyl groups can include mono- or polycyclic (e.g., having 2, 3 or 4 fused rings) ring systems as well as spirocycles. Example cycloheteroalkyl or heterocycloalkyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of cycloheteroalkyl or heterocycloalkyl are moieties that have one or more aromatic rings fused (i.e., having a bond in common with) to the nonaromatic heterocyclic ring, for example phthalimidyl, naphthalimidyl, and benzo derivatives of heterocycles. A cycloheteroalkyl or heterocycloalkyl group having one or more fused aromatic rings can be attached though either the aromatic or non-aromatic portion. Also included in the definition of cycloheteroalkyl or heterocycloalkyl are moieties where one or more ring-forming atoms are substituted by 1 or 2 oxo or sulfido groups. In some embodiments, the cycloheteroalkyl or heterocycloalkyl group has from 1 to about 20 carbon atoms, and in further embodiments from about 3 to about 20 carbon atoms. In some embodiments, the cycloheteroalkyl or heterocycloalkyl group contains 3 to about 20, 3 to about 14, 3 to about 7, or 5 to 6 ring-forming atoms. In some embodiments, the cycloheteroalkyl or heterocycloalkyl group has 1 to about 4, 1 to about 3, or 1 to 2 heteroatoms. In some embodiments, the cycloheteroalkyl or heterocycloalkyl group contains 0 to 3 double bonds. In some embodiments, the cycloheteroalkyl or heterocycloalkyl group contains 0 to 2 triple bonds.

In some embodiments, “halo” or “halogen” includes fluoro, chloro, bromo, and iodo.

In some embodiments, the term “substituted” refers to the replacement of a hydrogen moiety with a non-hydrogen moiety in a molecule or group. The term “mono-substituted” or “poly-substituted” means substituted with one or more than one substituent up to the valence of the substituted group. For example, a mono-substituted group can be substituted with 1 substituent, and a poly-substituted group can be substituted with 2, 3, 4, or 5 substituents. Generally when a list of possible substituents is provided, the substituents can be independently selected from that group.

In some embodiments, the term “reacting” is meant to refer to the bringing together of the indicated reagents in such a way as to allow their molecular interaction and chemical transformation according to the thermodynamics and kinetics of the chemical system. Reacting can be facilitated, particularly for solid reagents, by using an appropriate solvent or mixture of solvents in which at least one of the reagents is at least partially soluble. Reacting is typically carried out for a suitable time and under conditions suitable to bring about the desired chemical transformation.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

In the case of the compounds which contain an asymmetric carbon atom, the invention relates to the D form, the L form, and D,L mixtures and also, where more than one asymmetric carbon atom is present, to the diastereomeric forms. Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide—imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the term, “compound” or “conjugate,” as used herein, is meant to include all stereoisomers, geometric isomers, tautomers, and isotopes of the structures depicted.

In some embodiments, the conjugate of the invention is substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

In some embodiments, a “therapeutically effective amount” as used herein refers to the amount which provides a therapeutic effect for a given condition and administration regimen.

In some embodiments, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The “subject” used here refers to an animal or a human. In some embodiment, the term “subject” refers to a human.

### Compositions and Administration

In another aspect, the invention features a pharmaceutical composition comprising a DUPA-Indenoisoquinoline conjugate of the invention, and at least one pharmaceutically acceptable carrier.

A therapeutically effective dose of the DUPA-Indenoisoquinoline conjugates according to the invention is used, in addition to physiologically acceptable carriers, diluents and/or adjuvants for producing a pharmaceutical composition. The dose of the active conjugates can vary depending on the route of administration, the age and weight of the patient, the nature and severity of the diseases to be treated, and similar factors. The daily dose can be given as a single dose, which is to be administered once, or be subdivided into two or more daily doses, and is as a rule 0.001-2000 mg. Particular preference is given to administering daily doses of 0.1-500 mg, e.g. 0.1-100 mg.

Suitable administration forms are oral, parenteral, intravenous, transdermal, topical, inhalative, intranasal and sublingual preparations. Particular preference is given to using oral, parenteral, e.g. intravenous or intramuscular, intranasal, e.g. dry powder or sublingual preparations of the compounds according to the invention. The customary galenic preparation forms, such as tablets, sugar-coated tablets, capsules, dispersible powders, granulates, aqueous solutions, alcohol-containing aqueous solutions, aqueous or oily suspensions, syrups, juices or drops, are used.

Solid medicinal forms can comprise inert components and carrier substances, such as calcium carbonate, calcium phosphate, sodium phosphate, lactose, starch, mannitol, alginates, gelatine, guar gum, magnesium stearate, aluminium stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavorings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators. Examples of such additives are tartrate and citrate buffers, ethanol and sequestering agents (such as ethylenediaminetetraacetic acid and its nontoxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar agar, calcium phosphate, magnesium stearate, animal and vegetable fats, and solid high molecular weight polymers, such as polyethylene glycol.

Oily suspensions for parenteral or topical applications can be vegetable synthetic or semisynthetic oils, such as liquid fatty acid esters having in each case from 8 to 22 C atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 C atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil or soybean oil.

Suitable solvents, gelatinizing agents and solubilizers are water or watermiscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of Na lauryl sulphate, fatty alcohol ether sulphates, di-Na—N-lauryl-β-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions.

As indicated above, the DUPA-Indenoisoquinoline conjugates of the invention may be administered as a combination therapy with further active agents, e.g. therapeutically active compounds useful in the treatment of cancer, for example, prostate cancer, ovarian cancer, lung cancer, or breast cancer. For a combination therapy, the active ingredients may be formulated as compositions containing several active ingredients in a single dose form and/or as kits containing individual active ingredients in separate dose forms. The active ingredients used in combination therapy may be coadministered or administered separately.

### Pharmaceutical Methods

The DUPA-Indenoisoquinoline conjugates of the present invention contain topoisomerase I (Top 1) inhibitors, which can be released upon entry of a cell, e.g., a cancer cell, for example, a prostate cancer cell. It is therefore the invention that the DUPA-Indenoisoquinoline conjugates of the invention can be used for treating or preventing disorders caused by, associated with and/or accompanied by topoisomerase I (Top 1) in which inhibiting topoisomerase I is of value. The DUPA-Indenoisoquinoline conjugates of the present invention can be used to treat cancers known to be susceptible to topoisomerase I inhibitors, including, but not limited to, chronic lymphocytic leukemia, multiple myeloma, large cell anaplastic carcinomas, lung cancer, Ewing's sarcoma, non-Hodgkins lymphoma, breast cancer, colon cancer, stomach cancer, ovarian cancer, bladder cancer, malignant melanoma, and prostate cancer. In another aspect, the present invention relates to methods of inhibiting the growth of cancer cells which comprises contacting the cells with an effective amount of a DUPA-Indenoisoquinoline conjugate of the present invention to obtain inhibition of growth of the tumor cells while protecting normal cells from topoisomerase I inhibitor induced cytotoxicity. It is an embodiment of this invention, that the DUPA-Indenoisoquinoline conjugate of the invention can be used for the treatment of cancer, for example, prostate cancer, ovarian cancer, lung cancer, or breast cancer. In some embodiments, the DUPA-Indenoisoquinoline conjugate of the present invention can be used for the treatment of prostate cancer.

FIG. 1 depicts the general schematic representation of a ligand-drug conjugate: the tumor-targeting ligand (e.g., DUPA) is connected to a cytotoxic drug (e.g., Top1 inhibitor) via a peptide linker and a drug-release segment (e.g., carbonate linkage) that will allow a facile release of the free drug inside the target cell.

PSMA (also called folate hydrolase I or glutamate carboxypeptidase II) is a type II membrane glycoprotein that shows high affinity for the ligand 2-[3-(1,3-Dicarboxypropyl)-Ureido]Pentanedioic Acid (DUPA) (Kᵢ=8 nM, IC₅₀=47 nM) (Kozikowski, et al. J. Med. Chem. 2004, 47, 1729-1738; Kozikowski, et al. J. Med. Chem. 2001, 44, 298-301). Upon binding to a ligand (e.g., DUPA), PSMA undergoes endocytosis, unloads the ligand, and then recycles rapidly to the cell surface. PSMA has been found in all tumor stages and was shown to be up-regulated following androgen deprivation (Wang, et al., J. Cell. Biochem. 2007, 102, 571-579).

The DUPA-Indenoisoquinoline conjugates of the present invention include an indenoisoquinoline topoisomerase I (Top1) inhibitors which are conjugated to the ligand DUPA, which selectively binds to PSMA (Kularatne, et al., J. Med. Chem. 2010, 53, 7767-7777), thus improving cytotoxicity by allowing the drugs to enter prostate cancer cells more easily and selectively, enhancing their bioavailabilities and potencies while reducing adverse side effects to normal cells that lack PSMA. A suitable peptide linker was added as a spacer between the drug, for example, indenoisoquinoline inhibitors, and the DUPA ligand in order to (1) facilitate the binding of PSMA to the DUPA moiety, thus, preventing any possible intervention of the cytotoxic drug to the binding of PSMA and its ligand, and (2) improve the overall water solubility of the indenoisoquinoline Top1 inhibitors, whose limited solubility is among major drawbacks of this drug type in clinical development. Peptide was chosen as the linker for several reasons: (1) ease of synthesis, (2) flexibility in chemical modifications, (3) higher stability in various conditions (pH, temperature), and (4) being more biocompatible and less susceptible to immunogenic response because its building blocks are natural L-amino acids, which can be potentially used by the surrounding tissue upon peptide degradation.

The DUPA conjugation of indenoisoquinoline Top1 inhibitors are an effective method to safely and selectively deliver the indenoisoquinoline anticancer agents to prostate cancer cells. For example, the prostate-targeting ligand DUPA was linked to the potent and cytotoxic Top1 inhibitor 18 (IC₅₀ of 2.0 nM against 22RV1 cells) via a disulfide linker for drug release and a peptide linker that ensures the binding of DUPA to its receptor (PSMA) and improves the overall water solubility of the conjugate. In contrast to the free drug 18, the conjugate was not lethal at the effective dose tested (40 nmol/mouse). Further, experimental results indicated that the uptake of the DUPA conjugate 86 was mediated by PSMA, and that 86 dissolved easily in water at room temperature while free drug 5 exhibited poor aqueous solubility. Furthermore, the DUPA-targeting mechanism has also been attached to a ^{99m}Tc radioimaging agent that can be used in conjunction with the DUPA conjugate to locate and monitor response to therapy, and identify patients who are suitable for the DUPA-indenoisoquinoline Top1 inhibitor treatment (Kularatne, et al. Mol. Pharmaceutics 2009, 6, 780-789 and 790-800). Additionally, the unique characteristics of PSMA (expression level rises with tumor aggressiveness, and it is present at all tumor stages and is upregulated following androgen withdrawal) render it a useful therapeutic target for chemotherapy, which, along with the current conjugation approach, provides new and effective drugs for the treatment and cure of metastatic prostate cancer without causing unacceptable, dose-limiting toxic effects.

It will be appreciated by persons skilled in the art that the present invention is not limited by what has been particularly shown and described herein above. Rather the scope of the present invention includes both combinations and subcombinations of the various features described hereinabove as well as variations and modifications which would occur to persons skilled in the art upon reading the specification and which are not in the prior art.

The invention will be further illustrated with reference to the following illustrative examples, which are not intended to limit the scope of the invention in any manner.

### EXAMPLES

### General Methods

Solvents and reagents were purchased from commercial vendors and were used without further purification. Melting points were determined using capillary tubes with a Mel-Temp apparatus and were uncorrected. Infrared spectra were obtained as films on KBr pellets with CHCl₃ as the solvent, using a Perkin-Elmer 1600 series or Spectrum One FTIR spectrometer, and were baseline-corrected. ¹H NMR spectra were recorded at 300 or 500 MHz, using a Bruker ARX300 or Bruker Avance 500 spectrometers with a QNP probe or TXI 5 mm/BBO probe, respectively.

Mass spectral analyses were performed at the Purdue University Campus-Wide Mass Spectrometry Center. APCI-MS studies were carried out using an Agilent 6320 ion trap mass spectrometer. ESI-MS studies were performed using a FinniganMAT XL95 (FinniganMAT Corp., Bremen, Germany) mass spectrometer. The instrument was calibrated to a resolution of 10000 with a 10% valley between peaks using the appropriate polypropylene glycol standards. MALDI-MS studies were performed using an Applied Biosystems (Framingham, Mass.) Voyager DE PRO mass spectrometer. This instrument utilizes a nitrogen laser (337 nm UVlaser) for ionization with a time-of-flight mass analyzer. The matrix used for these samples was (R)-cyano-4-hydroxy cinnamic acid, and the peptide LHRH was used as an internal standard.

Analytical thin layer chromatography was carried out on Baker-flex silica gel IB2-F plastic-backed TLC plates. Compounds were visualized with both short and long wavelength UV light and ninhydrin staining unless otherwise specified. Silica gel flash column chromatography was performed using 40-63 μM flash silica gel. Solid-phase peptide synthesis (SPPS) was performed using a standard peptide synthesis apparatus (Chemglass, Vineland, N.J.).

All peptides and peptide conjugates were purified by preparative reverse-phase high-performance liquid chromatography (RP-HPLC; Waters, xTerra C₁₈ 10 μm; 19 mm×250 mm) and analyzed by analytical RP-HPLC (Waters 1525 binary HPLC pump with a Waters 2487 dual wavelength absorbance detector and an injection volume of 10 μL). A Sunrise C₁₈ 5 μM 100 Å reverse-phase column with dimensions of 15 cm×4.6 mm (ES Industries), was used for all analytical HPLC experiments. For purities estimated by HPLC, the major peak accounted for ≧95% of the combined total peak area when monitored by a UV detector at 254 nm unless otherwise specified. Liquid chromatography/mass spectrometry (LC/MS) analyses were obtained using a Waters micromass ZQ 4000 mass spectrometer coupled with a UV diode array detector. All yields refer to isolated compounds.

### General Procedure for IC₅₀ (Dose Dependence) Studies

22RV1 cells were seeded in 24-well (50000 cells/well) Falcon plates and allowed to form monolayers over a period of 24-48 h. The old medium was replaced with fresh medium (0.5 mL) containing increasing concentrations of drug (either targeted or non-targeted) and cells were incubated for an additional 2 h and 24 h (in the case of targeted drug) at 37° C. Cells were washed (3×0.5 mL) with fresh medium and incubated in fresh medium (0.5 mL) for another 66 h at 37° C. The spent medium in each well was replaced with fresh medium (0.5 mL) containing [³H]-thymidine (1 mCi/mL), and the cells were incubated for additional 4 h at 37° C. to allow [³H]-thymidine incorporation. The cells were then washed with medium (2×0.5 mL) and treated with 5% trichloroacetic acid (0.5 mL) for 10 min at room temperature. The trichloroacetic acid was replaced with 0.25 N NaOH (0.5 mL) and the cells were transferred to individual scintillation vials containing Ecolume scintillation cocktail (3.0 mL), mixed well to form homogeneous liquid, and counted in a liquid scintillation analyzer. IC₅₀ values were calculated by plotting %[³H]-thymidine incorporation versus log concentration of drugs (targeted and non-targeted) using in GraphPad Prism 4.

### In Vivo Experiment

Five to six week old male nu/nu mice (Harlan Laboratories) were maintained on a standard 12 h light-dark cycle and fed on normal mouse chow for the duration of the experiment. PSMA-positive prostate cancer 22RV1 cells (2×106 in 20% HC matrigel) were injected in the right shoulders of the mice. Tumors were measured in two perpendicular directions every two to three days with vernier calipers, and their volumes calculated as 0.5×L×W2 where L is the longest axis (in millimeters) and W is the axis perpendicular to L (in millimeters). Dosing solutions of the test compound were prepared in sterile saline and administered in mice via i.p injection. Mice were divided into two groups (5 mice/group) and treatments were initiated when the subcutaneous tumors reached ˜100 mm³ in volume. Each dose was given at 2 μmol/kg of the test compound in a volume of 100 μL of saline. As a measure of gross toxicity, mouse weights were also recorded at each dosing. Results were plotted by using Graph Pad Prism4.

### Example 1

### N-[4-(Benzyloxy)benzylidene]-3-bromo-1-propylamine (54)

3-Bromopropylamine hydrobromide (3.56 g, 16.2 mmol) was diluted in CHCl₃ (50 mL) and Et₃N (1.64 g, 16.2 mmol). The mixture was stirred for 5 min, and then compound 53 (3.00 g, 14.1 mmol) and Na₂SO₄ (4.02 g, 28.3 mmol) were added. The mixture was stirred at room temperature for 16 h, and then washed with H₂O (100 mL×3) and brine (100 mL). The organic layer was dried over anhydrous Na₂SO₄, filtered and concentrated to yield the product 54 as a pale yellow syrup (4.69 g, 100%+residual solvent). IR (film) 2839, 1645, 1605, 1509, 1246, 1166, 830 cm⁻¹; ¹H NMR (300 MHz, CDCl₃) δ6.26 (s, 1H), 7.68 (dd, J=1.8 and 6.9 Hz, 2H), 7.45-7.33 (m, 5H), 7.02 (dd, J=1.9 and 6.9 Hz, 2H), 5.11 (s, 2H), 3.74 (dt, J=0.9 and 6.2 Hz, 2H), 3.51 (t, J=6.6 Hz, 2H), 2.27 (m, 2H); ESIMS m/z (rel. intensity) 332/334 (MH⁺, 100/97).

### Example 2

### cis-4-Carboxy-3,4-dihydro-N-(3-bromopropyl)-3-[4-(benzyloxy)phenyl]-7-nitro-1(2H)-isoquinolone (55)

Schiff base 54 (4.69 g, 14.1 mmol) was diluted in CHCl₃ (50 mL) at 0° C., and the anhydride 58 (2.80 g, 13.5 mmol) was added. The red mixture was stirred at 0° C. for 2 h, and then warmed up to room temperature and stirring was continued for 2 h. The creamy orange mixture was filtered, and the residue was washed with CHCl₃ to provide the product 55 as an off-white solid (5.18 g, 71%): mp 140-141° C. IR (film) 3076, 1727, 1630, 1525, 1347, 1187, 738 cm⁻¹; ¹H NMR (300 MHz, DMSO-d₆) δ8.71 (d, J=2.6 Hz, 1H), 8.39 (dd, J=2.6 and 6.0 Hz, 1H), 7.92 (d, J=8.2 Hz, 1H), 7.40-7.30 (m, 5H), 6.92-6.83 (m, 4H), 5.19 (d, J=6.4 Hz, 1H), 5.03 (d, J=6.3 Hz, 1H), 4.98 (s, 2H), 3.90 (m, 1H), 3.59 (m, 2H), 3.03 (m, 1H), 2.16 (m, 1H), 2.04 (m, 1H); ESIMS m/z (rel. intensity) 415 ([MH—COOH—Br]⁺, 100); HRESIMS calcd for MH⁺: 539.0818. found: 539.0812.

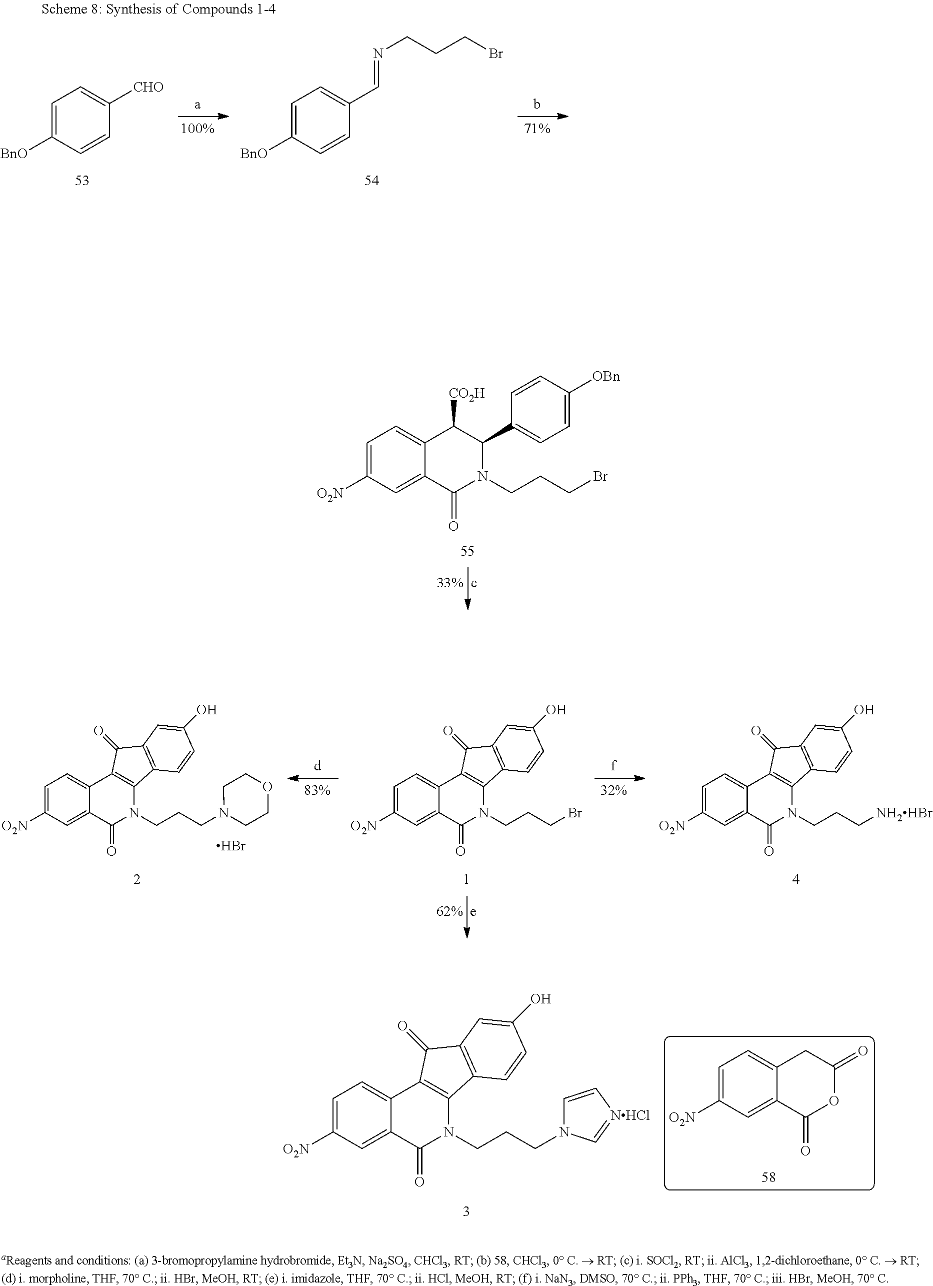

### Example 3

### 6-(3-Bromopropyl)-9-hydroxy-3-nitro-5H-indeno[1,2-c]isoquinoline-5,11(6H)-dione (1)

Cis acid 56 (0.50 g, 0.93 mmol) was diluted in SOCl₂ (50 mL) and stirred for 16 h at room temperature. The resulted yellow solution was evaporated to dryness. The yellow syrup was diluted in 1,2-dichloroethane (50 mL) at 0° C. and stirred for 15 min, followed the addition of AlCl₃ (0.25 g, 1.85 mmol). The black mixture was heated at reflux for 2 h, and then evaporated to dryness. The remaining residue was diluted with CHCl₃ (100 mL), and washed with HCl 6 N (100 mL), H₂O (100 mL×3) and brine (100 mL). The organic layer was dried over anhydrous Na₂SO₄, filtered and concentrated, adsorbed onto SiO₂, and purified with flash column chromatography (SiO₂), eluting with CHCl₃ to provide the product 1 as a red solid (57 mg, 15%): mp 281-283 (dec) ° C. IR (film) 3273, 1659, 1613, 1531, 1345, 1270, 755 cm⁻¹; ¹H NMR (300 MHz, DMSO-d₆) δ10.82 (s, 1H), 8.83 (d, J=2.1 Hz, 1H), 8.61 (d, J=9.0 Hz, 1H), 8.51 (d, J=9.2 Hz, 1H), 7.74 (d, J=8.6 Hz, 1H), 6.99 (s, 1H), 6.89 (d, J=8.6 Hz, 1H), 4.54 (m, 2H), 3.78 (t, J=6.3 Hz, 2H), 2.33 (m, 2H); ESIMS m/z (rel. intensity) 428/430 (M⁺, 99/100); HRESIMS calcd for M⁺: 428.0008. found: 428.0000.

### Example 4

### 9-Hydroxy-6-(3-morpholinopropyl)-3-nitro-5H-indeno[1,2-c]isoquinoline-5,11(6H)-dione Hydrobromide (2)

Phenol 1 (50 mg, 0.12 mmol) was diluted in THF (30 mL), followed by the addition of K₂CO₃ (83 mg, 0.58 mmol) and morpholine (51 mg, 0.58 mmol). The red solution was heated at 70° C. for 16 h. The cooled solution was diluted with aqueous HBr (48% wt, 20 mL) and stirred at room temperature for 3 h. The deep red solution was then diluted with CHCl₃ (10 mL) and acetone (10 mL) and concentrated. The dilution and concentration were repeated for 3 times to remove HBr. The final concentrate was filtered through an HPLC membrane filter, and the residue was washed with CHCl₃ to provide the product 2 as a deep brown solid (49 mg, 83%): mp 295-297 (dec) ° C. ¹H NMR (300 MHz, DMSO-d₆) δ10.87 (s, 1H), 9.53 (br s, 1H), 8.86 (d, J=2.4 Hz, 1H), 8.66 (d, J=9.0 Hz, 1H), 8.56 (dd, J=2.4 and 6.5 Hz, 1H), 7.70 (d, J=8.4 Hz, 1H), 7.04 (d, J=2.4 Hz, 1H), 6.93 (dd, J=2.3 and 6.0 Hz, 1H), 4.52 (m, 2H), 3.98 (m, 2H), 3.64 (m, 2H), 3.38 (m, 4H), 3.08 (m, 2H), 2.21 (m, 2H); ESIMS (positive mode) m/z (rel intensity) 436 (MH⁺, 100); HRESIMS calcd for MH⁺: 436.1509. found: 436.1508.

### Example 5

### 6-(3-(1H-Imidazol-1-yl)propyl)-7-hydroxy-3-nitro-5H-indeno[1,2-c]isoquinoline-5,11(6H)-dione hydrochloride (3)

Bromide 1 (70 mg, 0.16 mmol) was diluted in 1,4-dioxane (20 mL), followed by the addition of NaI (122 mg, 0.815 mmol) and imidazole (67 mg, 0.98 mmol). The red mixture was heated at 70° C. for 16 h, and then concentrated to a volume of 10 mL. The mixture was filtered, and the residue was washed with acetone and CHCl₃ to provide the neutral compound as a deep red solid. The crude product was diluted and stirred in methanolic HCl 3 N (20 mL) at room temperature for 5 h. The mixture was concentrated and filtered. The residue was washed with CHCl₃ to afford the product 3 as a brown solid (42.6 mg, 62%): mp 323-325° C. (dec). IR (film) 1668, 1611, 1503, 1428, 1334, 1263 cm⁻¹; ¹H NMR (300 MHz, DMSO-d₆) δ9.38 (d, J=9.3 Hz, 1H), 8.97 (d, J=2.5 Hz, 1H), 8.67 (dd, J=2.5 and 6.7 Hz, 1H), 7.85 (d, J=7.7 Hz, 1H), 7.76 (t, J=7.4 Hz, 1H), 7.56 (d, J=8.0 Hz, 1H), 7.38 (t, J=8.5 Hz, 1H), 4.37 (m, 2H), 4.14 (m, 2H); ESIMS (positive mode) m/z (rel. intensity) 417 (MH⁺, 100); HRESIMS calcd for MH⁺: 417.1199. found: 417.1202; HPLC purity: 100% (MeOH, 100%), 100% (MeOH—H₂O, 70:30).

### Example 6

### 6-(3-Aminopropyl)-9-hydroxy-3-nitro-5H-indeno[1,2-c]isoquinoline-5,11(6H)-dione Hydrobromide (4)

Bromide 1 (100 mg, 0.23 mmol) was diluted in DMSO (20 mL), followed by the addition of NaN₃ (75 mg, 1.15 mmol). The mixture was heated at 70° C. for 16 h and extracted with CHCl₃ (50 mL×2). The extract was washed with H₂O (100 mL×4) and brine (100 mL). The organic layer was dried over anhydrous Na₂SO₄, filtered and concentrated to provide the crude azide. The compound was diluted in THF (20 mL), followed by the addition of PPh₃ (181 mg, 0.69 mmol), and the mixture was heated at reflux for 16 h. The cooled deep brown solution was diluted with 3 M methanolic HBr (20 mL), and stirring was continued at reflux for 4 h. The bright red is solution was evaporated, re-diluted in CHCl₃ (10 mL), and let sit for 16 h at 0° C., during which a precipitate formed. The solution was then filtered through an HPLC filter paper, and the residue was washed thoroughly with CHCl₃ to provide the product 4 (33.1 mg, 32%) as a deep red solid: mp 360-362° C. (dec). ¹H NMR (300 MHz, DMSO-d₆) δ 10.83 (s, 1H), 8.83 (d, J=2.3 Hz, 1H), 8.61 (d, J=9.0 Hz, 1H), 8.52 (dd, J=2.5 and 6.4 Hz, 1H), 7.74-7.67 (m, 4H), 6.99 (d, J=2.3 Hz, 1H), 6.91 (dd, J=2.2 and 6.1 Hz, 1H), 4.52 (t, J=7.0 Hz, 2H), 2.99 (m, 2H), 2.08 (m, 2H); APCIMS m/z (rel. intensity) 366 ([MH—NH₃]⁺, 100); HRMSESI calcd for MH⁺: 366.1090. found: 366.1082.

### Example 7

### Compounds 5-11, 15, and 17-19

Compound 5-11, 15, and 17-19 were synthesized based on the procedures reported by Morrell et al. (Morrell, et al., J. Med. Chem. 2006, 49, 7740-7753; Morrell, et al., J. Med. Chem. 2007, 50, 4419-4430; and Morrell, et al., J. Med. Chem. 2007, 50, 4388-4404). Purities of biologically tested indenoisoquinoline amine hydrochlorides 69-79 were ≧95% by HPLC.

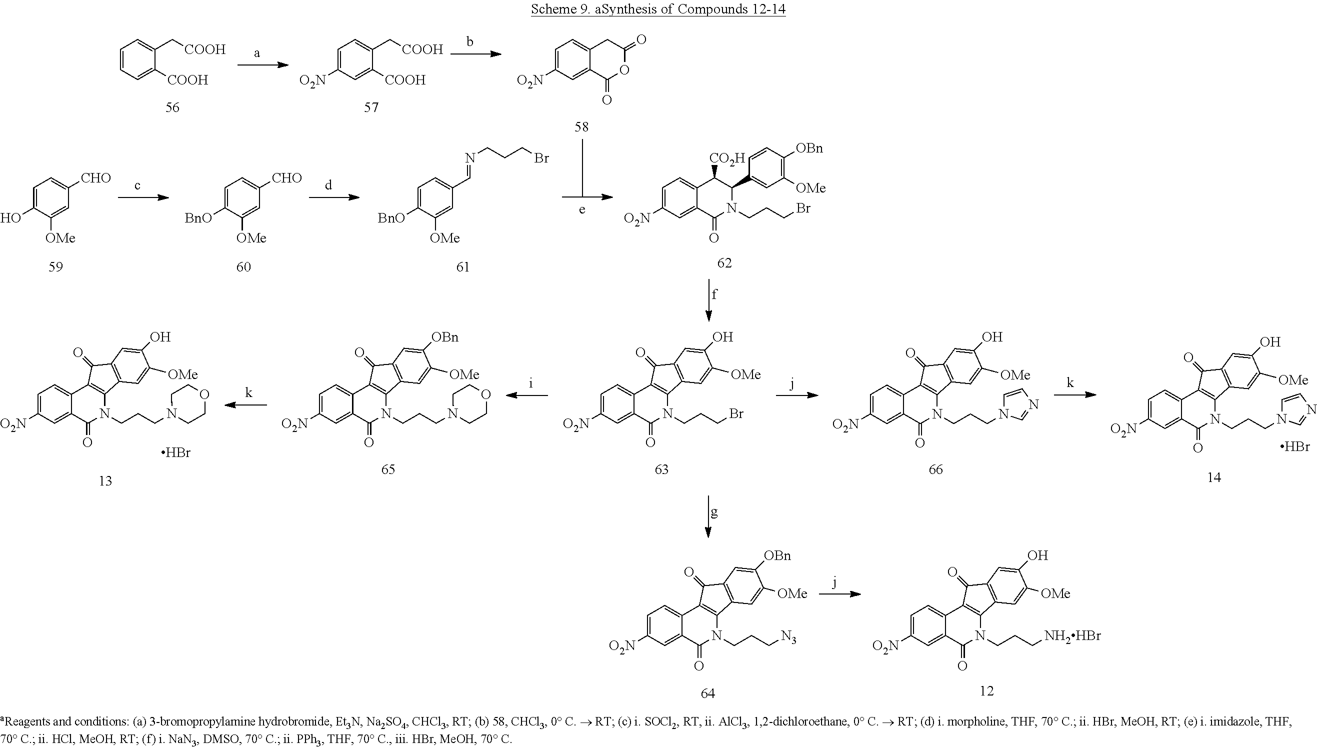

### Example 8

### 4-Nitrohomophthalic Anhydride (58)

(Whitmore, et al., J. Am. Chem. Soc. 1944, 66, 1237-1240)

Diacid 6 (8.83 g, 39.2 mmol) was diluted in acetyl chloride (30 mL), and the mixture was stirred at reflux for 2 h, and then AcCl was evaporated. The remaining solution was filtered and washed slightly with CHCl₃ to provide the product 58 as a white solid (5.75 g, 71%): mp 147-148° C. (lit. (Whitmore, et al., J. Am. Chem. Soc. 1944, 66, 1237-1240), 154-155° C.). ¹H NMR (300 MHz, DMSO-d₆) δ8.66 (s, 1H), 8.55 (d, J=8.1 Hz, 1H), 7.73 (d, J=7.9 Hz, 1H), 4.41 (s, 2H).

### Example 9

### Benzylvanillin (60)

(Guthrie, et al., Can. J. Chem. 1955, 33, 729-742)

Benzyl bromide (5.90 g, 34.5 mmol) was added to a solution of vanillin 59 (5.00 g, 32.9 mmol) in DMF (50 mL), followed by the addition of K₂CO₃ (9.08 g, 65.7 mmol). The yellow mixture was stirred at room temperature for 2 h, and then poured into a solution of Et₂O—H₂O (200 mL, 1:1) and stirred for 5 min. The ethereal layer was separated. The aqueous layer was extracted with Et₂O (50 mL×2). The combined organic extract was washed with H₂O (50 mL×3) and brine (50 mL), and dried over anhydrous Na₂SO₄, filtered and concentrated to obtain a crude residue, which was washed with hexane to furnish the pure product 60 as a white solid (7.91 g, 99%): mp 49-51° C. (lit. (Guthrie, et al., Can. J. Chem. 1955,33, 729-742), 61° C.). ¹H NMR (300 MHz, CDCl₃) δ59.84 (s, 1H), 7.44-7.36 (m, 7H), 7.00 (d, J=8.2 Hz, 1H), 5.25 (s, 2H), 3.95 (s, 3H).

### Example 10

### N-[4′-(Benzyloxy)-3′-methoxybenzylidene]-3-bromopropan-1-amine (61)

3-Bromopropylamine hydrobromide (3.12 g, 14.2 mmol) was diluted in CHCl₃ (10 mL). Benzylvanillin 60 (3.00 g, 12.4 mmol) was dissolved in CHCl₃ (10 mL) and added slowly to the amine solution. Upon the addition of Et₃N (1.39 g, 13.6 mmol), the mixture turned clear. Na₂SO₄ (3.52 g, 24.8 mmol) was added, and the mixture was stirred at room temperature for 16 h, and then diluted to 50 mL with CHCl₃ and washed with H₂O (100 mL×3) and brine (100 mL). The organic layer was dried over anhydrous Na₂SO₄, filtered and concentrated to provide the product 61 as a yellow syrup (4.49 g, 100%+residual solvent). IR (film) 2937, 2841, 1646, 1602, 1587, 1512, 1456, 1415, 1270, 1233, 743 cm⁻¹; ¹H NMR (300 MHz, CDCl₃) 458.22 (s, 1H), 7.45-7.30 (m, 6H), 7.10 (dd, J=1.7 and 6.4 Hz, 1H), 6.90 (d, J=8.2 Hz, 1H), 5.20 (s, 2H), 3.95 (s, 3H), 3.74 (t, J=6.1 Hz, 2H), 3.51 (t, J=6.5 Hz, 2H), 2.27 (m, 2H).

### Example 11

### cis-4-Carboxy-3,4-dihydro-N-(3-bromopropyl)-3-[4-(benzyloxy)-3-methoxyphenyl]-7-nitro-1(2H)-isoquinolone (62)

Schiff base 61 (7.48 g, 20.7 mmol) was diluted in CHCl₃ (50 mL) and cooled to 0° C. for 10 min, followed the addition of anhydride 58 (4.28 g, 20.7 mmol). The red mixture was stirred at 0° C. for 2 h, and then at room temperature for 3 h more. The mixture was filtered, and the residue was washed with CHCl₃ to afford the product 62 as a white solid (7.55 g, 64%): mp 145-146° C. IR (film) 3079, 1748, 1621, 1520, 1493, 1418, 1349, 1177, 755 cm⁻¹; ¹H NMR (300 MHz, CDCl₃) δ9.06 (d, J=2.4 Hz, 1H), 8.36 (dd, J=2.5 and 6.0 Hz, 1H), 7.87 (d, J=8.8 Hz), 7.35-7.28 (m, 5H), 6.71 (d, J=8.9 Hz, 1H), 6.50 (m, 2H), 5.13 (d, J=6.4 Hz, 1H), 5.04 (s, 2H), 4.80 (d, J=6.4 Hz, 1H), 4.04 (m, 1H), 3.63 (s, 3H), 3.48 (m, 2H), 3.28 (m, 1H), 2.31 (m, 1H), 2.18 (m, 1H); ESI-MS m/z (rel intensity) 569/571 ([MH]⁺, 27/28); HRMS (+ESI) calcd for MH⁺: 569.0923. found: 569.0932.

### Example 12

### 9-(Benzyloxy)-6-(3-bromopropyl)-8-methoxy-3-nitro-5H-indeno[1,2-c]isoquinoline-5,11(6H)-dione (63)

cis acid 62 (1.50 g, 2.63 mmol) was diluted in SOCl₂ (50 mL) and the mixture was stirred at room temperature for 4 h. The red solution was evaporated to dryness, and the residue was diluted with CHCl₃ (50 mL) and quenched slowly with sat. NaHCO₃ (100 mL). The mixture was stirred at room temperature for 10 min, and the two layers were separated. The aqueous layer was extracted with CHCl₃ (50 mL). The combined organic layers were washed with H₂O (100 mL×3) and brine (100 mL), and then dried over anhydrous Na₂SO₄, filtered and adsorbed onto SiO₂, purified by flash column chromatography (SiO₂), eluting with CHCl₃ to provide the product 63 as a reddish brown solid (228 mg, 16%): mp 218-220(dec) ° C. IR (film) 1677, 1611, 1504, 1427, 1336, 1300, 746 cm⁻¹; ¹H NMR (300 MHz, CDCl₃) δ9.15 (d, J=2.4 Hz, 1H), 8.78 (d, J=9.0 Hz, 1H), 8.47 (dd, J=2.5 and 6.7 Hz, 1H), 7.44-7.36 (m, 6H), 7.31 (d, J=1.8 Hz, 1H), 5.26 (s, 2H), 4.71 (t, J=7.0 Hz, 2H), 4.06 (s, 3H), 3.74 (t, J=5.7 Hz, 2H), 2.51 (m, 2H); ESI-MS m/z (rel intensity) 549/551 ([MH]⁺, 42/53); HRMS (+ESI) calcd for MH⁺: 549.0661. found: 549.0672.

### Example 13

### 6-(3-Azidopropyl)-9-(benzyloxy)-8-methoxy-3-nitro-5H-indeno[1,2-c]isoquinoline-5,11(6H)-dione (64)

Compound 63 (150 mg, 0.273 mmol) and NaN₃ (178 mg, 2.73 mmol) were diluted in DMSO (50 mL) and heated at 70° C. for 15 h. The red solution was diluted with CHCl₃ (100 mL), washed with H₂O (100 mL×4) and brine (100 mL). The organic layer was dried over anhydrous Na₂SO₄, filtered and concentrated, adsorbed onto SiO₂ and purified by flash column chromatography (SiO₂), eluting with CHCl₃ to provide the product 64 as a deep red solid (36.2 mg, 26%): mp 205-207(dec) ° C. IR (film) 2090, 1673, 1610, 1579, 1502, 1427, 847 cm⁻¹; ¹H NMR (300 MHz, CDCl₃) δ9.14 (d, J=2.3 Hz, 1H), 8.75 (d, J=9.0 Hz, 1H), 8.45 (dd, J=2.3 and 6.7 Hz, 1H), 7.48-7.35 (m, 5H), 7.28 (s, 1H), 5.26 (s, 2H), 4.61 (t, J=6.9 Hz, 2H), 4.07 (s, 3H), 3.79 (t, J=5.7 Hz, 2H), 2.16 (m, 2H); ESI-MS m/z (rel. intensity) 512 (MH⁺, 100); HRMS (+ESI) calcd for MH⁺: 512.1570. found: 512.1576.

### Example 14

### 6-(3-Aminopropyl)-9-hydroxy-8-methoxy-3-nitro-5H-indeno[1,2-c]isoquinoline-5,11(6H)-dione hydrobromide (12)

Compound 64 (30 mg, 0.059 mmol) was diluted in benzene (50 mL), and triethylphosphite (29.2 mg, 0.176 mmol) was added. The mixture was heated at reflux for 16 h, and then allowed to cool to room temperature. Aqueous HBr (48% wt, 30 mL) was added, and the reaction mixture was heated at 70° C. for 5 h, during which it turned to a brown/red emulsion. The cooled mixture was concentrated to remove benzene and HBr. The concentrate was then diluted with acetone (10 mL) and concentrated again. This procedure was repeated three times. The final mixture was filtered under vacuum through an HPLC filter, and the residue was washed with CHCl₃ and acetone to provide the desired product 12 as a brown solid (26.0 mg, 93%): mp 285-287(dec) ° C. IR (film) 3243, 2848, 1705, 1641, 1614, 1562, 1488, 1336, 1207, 1133, 868 cm⁻¹; ¹H NMR (300 MHz, CDCl₃) δ10.41 (s, 1H), 8.83 (d, J=2.3 Hz, 1H), 8.60 (d, J=9.0 Hz, 1H), 8.51 (dd, J=2.5 and 6.5 Hz, 1H), 7.74 (br s, 3H), 7.19 (s, 1H), 7.03 (s, 1H), 4.58 (m, 2H), 3.98 (s, 3H), 3.01 (m, 2H), 2.14 (m, 2H); ESI-MS m/z (rel. intensity) 396 (MH⁺, 100); HRMS (+ESI) calcd for MH⁺: 396.1196. found: 396.1199; HPLC purity: 100% (MeOH, 100%), 98.6% (MeOH—H₂O, 90:10). Anal. Calcd for C₂₀H₁₈BrN₃O₆: C, 50.44; H, 3.81; N, 8.82. Found: C, 50.13; H, 3.75; N, 8.59.

### Example 15

### 9-(Benzyloxy)-8-methoxy-6-(3-morpholinopropyl)-3-nitro-5H-indeno[1,2-c]isoquinoline-5,11[6H]-dione (65)

Compound 63 (320 mg, 0.58 mmol) was diluted in anhydrous DMF (30 mL) and NaI (523 mg, 3.49 mmol) was added. The mixture was heated at 70° C. for 30 min, and then morpholine (304 mg, 3.49 mmol) was added, and heating was continued for 2 h. The solution was stirred at room temperature for 14 h, and then diluted with H₂O (100 mL) and extracted with CHCl₃ (50 mL×3). The extract was washed with H₂O (100 mL×5) and brine (100 mL), and then dried over anhydrous Na₂SO₄, filtered and adsorbed onto SiO₂, and purified by flash column chromatography (SiO₂), eluting with a gradient of 2% to 4% MeOH in CHCl₃ to provide the product 65 as a brown solid (140 mg, 44%): mp 233-234(dec) ° C. IR (film) 1673, 1612, 1557, 1507, 1428, 1333, 1300, 667 cm⁻¹; ¹H NMR (300 MHz, CDCl₃) δ9.15 (d, J=2.5 Hz, 1H), 8.75 (d, J=9.0 Hz, 1H), 8.45 (dd, J=2.4 and 6.5 Hz, 1H), 7.47-7.35 (m, 5H), 7.31 (s, 1H), 7.21 (s, 1H), 5.25 (s, 2H), 4.63 (t, J=7.3 Hz, 2H), 4.01 (s, 3H), 3.66 (m, 4H), 2.60 (t, J=6.7 Hz, 2H), 2.46 (m, 4H), 2.14 (m, 2H); ESI-MS m/z (rel intensity) 556 ([MH]⁺, 100); HRMS (+ESI) calcd for MH⁺: 556.2084. found: 556.2076.

### Example 16

### 9-Hydroxy-8-methoxy-6-(3-morpholinopropyl)-3-nitro-5H-indeno[1,2-c]isoquinoline-5,11(6H)-dione hydrobromide (13)

Compound 65 (50 mg, 0.090 mmol) was diluted with aqueous HBr (48% wt, 35 mL) and heated at 70° C. for 5 h, during which it turned to a black emulsion. The cooled mixture was concentrated to remove HBr. The concentrate was diluted with acetone (10 mL) and concentrated again. This procedure was repeated three times. The final mixture was filtered under vacuum, and the residue was washed with CHCl₃ and acetone to provide the desired product 13 as a black solid (47.5 mg, 97%): mp>400° C. IR (film) 3206, 1697, 1641, 1614, 1558, 1506, 1427, 1335, 792 cm⁻¹; ¹H NMR (300 MHz, CDCl₃) δ10.45 (s, 1H), 9.49 (s, 1H), 8.87 (s, 1H), 8.65 (d, J=8.8 Hz, 1H), 8.55 (d, J=8.2 Hz, 1H), 7.23 (s, 1H), 7.08 (s, 1H), 4.63 (m, 2H), 4.00-3.95 (m, 7H), 3.61 (m, 4H), 3.10 (m, 2H), 2.28 (m, 2H); ESI-MS m/z (rel. intensity) 447 (MH⁺, 89); HRMS (+ESI) calcd for MH⁺: 447.1305. found: 447.1303; HPLC purity: 100% (MeOH, 100%), 97.8% (MeOH—H₂O, 90:10). Anal. Calcd for C₂₄H₂₄BrN₃O₇.1H₂O: C, 52.59; H, 4.45; N, 7.67. Found: C, 52.28; H, 4.24; N, 7.30.

### Example 17

### 6-(3-(1H-Imidazol-1-yl)propyl)-9-(benzyloxy)-8-methoxy-3-nitro-5H-indeno[1,2-c]isoquinoline-5,11(6H)-dione (66)

Compound 63 (100 mg, 0.182 mmol) was diluted in anhydrous DMF (30 mL) and NaI (273 mg, 1.82 mmol) was added. The mixture was heated at 70° C. for 30 min, and then imidazole (124 mg, 1.82 mmol) was added and heating was continued for 16 h. The deep red solution was diluted with H₂O (100 mL) and extracted with CHCl₃ (50 mL×3). The extract were washed with H₂O (100 mL×5) and brine (200 mL), dried over anhydrous Na₂SO₄, filtered and adsorbed onto SiO₂, and purified by flash column chromatography (SiO₂), eluting with 4% MeOH in CHCl₃, to provide the product 66 as a brown solid (36.2 mg, 37%): mp 235-236(dec) ° C. IR (film) 1662, 1612, 1555, 1424, 1291, 746 cm⁻¹; ¹H NMR (300 MHz, CDCl₃) δ9.17 (d, J=2.3 Hz, 1H), 8.77 (d, J=9.0 Hz, 1H), 8.48 (dd, J=2.4 and 6.6 Hz, 1H), 7.61 (s, 1H), 7.46-7.30 (m, 5H), 7.12 (s, 1H), 7.05 (s, 1H), 6.85 (s, 1H), 5.24 (s, 2H), 4.61 (t, J=6.8 Hz, 2H), 4.27 (t, J=6.5 Hz, 2H), 3.86 (s, 3H), 2.42 (m, 2H); ESI-MS m/z (rel. intensity) 537 (MH⁺, 100); HRMS (+ESI) calcd for MH⁺: 537.1774. found: 537.1784.

### Example 18

### 6-(3-(1H-Imidazol-1-yl)propyl)-9-hydroxy-8-methoxy-3-nitro-5H-indeno[1,2-c]isoquinoline-5,11(6H)-dione hydrobromide (14)

Compound 66 (50 mg, 0.093 mmol) was diluted with aqueous HBr (48% wt, 35 mL) and heated at 70° C. for 5 h, during which it turned to a brown emulsion. The cooled mixture was concentrated to remove HBr. The concentrate was then diluted with acetone (10 mL) and concentrated again. This procedure was repeated three times. The final mixture was filtered under vacuum, and the residue was washed with CHCl₃ and acetone to provide the desired product 14 as a pale brown solid (24.6 mg, 50%): mp>400° C. IR (film) 3398, 1680, 1609, 1557, 1492, 1429, 1385, 1338, 859 cm⁻¹; ¹H NMR (300 MHz, CDCl₃) δ10.43 (s, 1H), 9.11 (s, 1H), 8.86 (s, 1H), 8.65 (d, J=9.2 Hz, 1H), 8.54 (d, J=6.5 Hz, 1H), 7.83 (s, 1H), 7.68 (s, 1H), 7.23 (s, 1H), 7.08 (s, 1H), 4.60 (m, 2H), 4.37 (m, 2H), 3.97 (s, 3H), 2.50 (m, 2H, under the water peak); ESI-MS m/z (rel. intensity) 466 (MH⁺, 100); HRMS (+ESI) calcd for MH⁺: 466.1614. found: 466.1618; HPLC purity: 100% (MeOH, 100%), 96.7% (MeOH—H₂O, 90:10). Anal. Calcd for C₂₃H₁₉BrN₄O₆.0.5H₂O: C, 51.51; H, 3.76; N, 10.45. Found: C, 51.33; H, 3.46; N, 10.30.

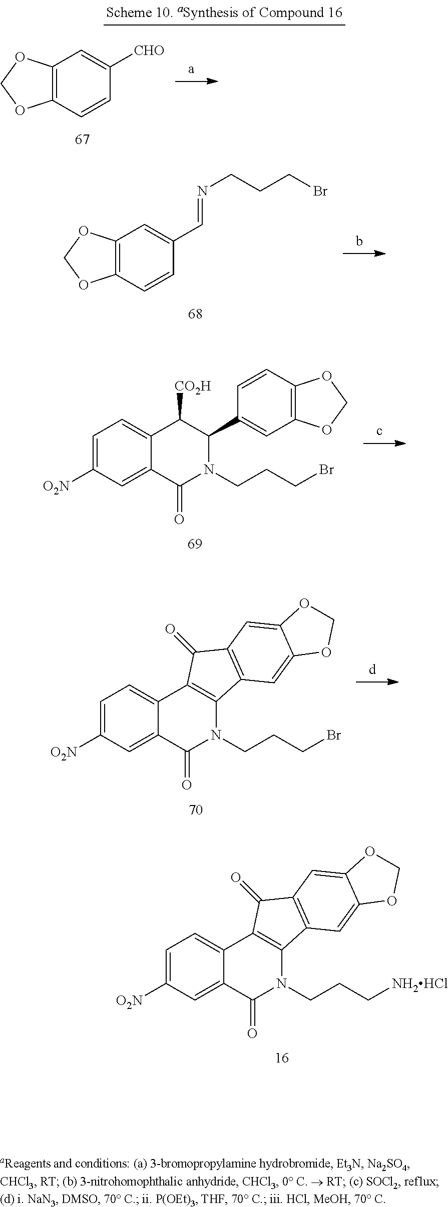

### Example 19

### 3-Bromo-N-(3,4-Methylenedioxybenzylidene)propan-1-amine (68)

3-Bromopropylamine hydrobromide (1.82 g, 8.33 mmol) was diluted in CHCl₃ (30 mL) and Et₃N (1.01 g, 9.99 mmol). The mixture was stirred until the salt dissolved completely, and then piperonal 67 (1.00 g, 6.66 mmol) and Na₂SO₄ (1.89 g, 13.3 mmol) were added. The mixture was stirred at room temperature for 16 h, diluted with CHCl₃ (100 mL), and then washed with H₂O (100 mL×3) and brine (100 mL). The organic layer was dried over anhydrous Na₂SO₄, filtered and concentrated to yield the product 68 as a pale yellow syrup (1.80 g, 100%). IR (film) 2897, 1643, 1605, 1447, 1253, 1037, 809 cm⁻¹; ¹H NMR (300 MHz, CDCl₃) δ8.21 (s, 1H), 7.34 (d, J=1.4 Hz, 1H), 7.12 (dd, J=1.5 and 6.4 Hz, 1H), 6.84 (d, J=7.9 Hz, 1H), 6.01 (s, 2H), 3.73 (dt, J=1.1 and 5.3 Hz, 2H), 3.51 (t, J=6.5 Hz, 2H), 2.28 (m, 2H); ESIMS m/z (rel. intensity) 270/272 (MH⁺, 100/93).

### Example 20

### cis-N-(3-Bromopropyl)-4-carboxy-3,4-dihydro-3-(3,4-methylenedioxyphenyl)-7-nitro-1(2H)-isoquinolone (69)

Schiff base 68 (1.80 g, 6.66 mmol) was diluted in CHCl₃ (50 mL) at 0° C., and 4-nitrohomophthalic anhydride 58 (1.38 g, 6.66 mmol) was added. The mixture was stirred at 0° C. for 2 h and then at room temperature for 2 h. The cloudy mixture was filtered, and the residue was washed with CHCl₃ to provide the crude acid 69 as an off-white solid (1.56 g, 49%): mp 167-168° C. ¹H NMR (300 MHz, DMSO-d₆) δ8.61 (d, J=2.4 Hz, 1H), 8.27 (dd, J=2.5 and 5.8 Hz, 1H), 7.49 (d, J=7.9 Hz, 1H), 6.78 (d, J=8.0 Hz, 1H), 6.68 (d, J=1.6 Hz, 1H), 6.48 (d, J=6.5 Hz, 1H), 5.94 (s, 2H), 5.05 (d, J=4.8 Hz, 1H), 4.10 (m, 1H), 3.77 (m, 1H), 3.60 (t, J=6.8 Hz, 2H), 2.96 (m, 1H), 2.48 (m, 2H); ESIMS m/z (rel. intensity) 477/479 (MH⁺, 92/98).

### Example 21

### 6-(3-Bromopropyl)-5,6-dihydro-8,9-methylenedioxy-3-nitro-5,11-dioxo-11H-indeno[1,2-c]isoquinoline (70)

Acid 69(1.00 g, 2.10 mmol) was heated in SOCl₂ (neat, 30 mL) for 1 h. The cooled grape-colored solution was evaporated to dryness, and the residue was triturated with ether, filtered and washed with ether to provide the product 70 as a brown solid (0.18 g, 19%): mp 260-263° C. (dec). The crude product was subjected to the next reaction without further purification.

### Example 22

### 6-(3-Aminopropyl)-5,6-dihydro-8,9-methylenedioxy-3-nitro-5,11-dioxo-11H-indeno[1,2-c]isoquinoline Hydrochloride (16)

Bromide 70(100 mg, 0.22 mmol) and NaN₃ (71 mg, 1.1 mmol) were heated in DMSO (25 mL) at 70° C. for 1 h. The cooled solution was diluted in H₂O (100 mL) and extracted with CHCl₃ (75 mL). The extract was washed with H₂O (100 mL×4) and brine (100 mL). The organic layer was dried over anhydrous Na₂SO₄, filtered and concentrated, adsorbed onto SiO₂ and purified with flash column chromatography (SiO₂), eluting with CHCl₃ to provide the intermediate azide. The azide and P(OEt)₃ (109 mg, 0.66 mmol) were diluted and heated in benzene (20 mL) at 70° C. for 16 h. The cooled solution was then diluted with 3 N HCl in methanol (30 mL) and heated at reflux for 2 h. The resulting solution was evaporated to dryness. The residue was triturated with acetone, filtered and washed with acetone to provide the product 16 as a brown solid (69.3 mg, 74%): mp 294-296° C. (dec). ¹H NMR (300 MHz, DMSO-d₆) δ8.80 (s, 1H), 8.57 (d, J=9.2 Hz, 1H), 8.50 (d, J=9.0 Hz, 1H), 7.81 (br s, 3H), 7.50 (s, 1H), 7.20 (s, 1H), 6.24 (s, 2H), 4.50 (m, 2H), 2.97 (m, 2H), 2.08 (m, 2H); HPLC purity: 98.5% (MeOH, 100%).

### Example 23

### 2-(Pyridin-2-yldisulfanyl)ethyl {3-[3-Nitro-5,11-dioxo-5,11-dihydro-6H-indeno[1,2-c]isoquinolin-6-yl]propyl}carbamate (71)

Compound 5 (44 mg, 0.11 mmol) was dissolved in CH₂Cl₂ (5 mL), followed by the addition of carbonate 27 (53 mg, 0.14 mmol), DMAP (14 mg, 0.11 mmol), and Et₃N (115 mg, 1.1 mmol). The mixture was stirred at room temperature for 16 h, and was then loaded directly onto a SiO₂ column and purified by flash column chromatography, eluting with CHCl₃, to provide the product 71 as an orange solid (42.1 mg, 66%): mp 220-221° C. IR (film) 3314, 1694, 1664, 1558, 1500, 1340, 767 cm⁻¹; ¹H NMR (300 MHz, DMSO-d₆) δ8.88 (s, 1H), 8.72 (d, J=8.9 Hz, 1H), 8.57 (d, J=10.9 Hz, 1H), 8.43 (m, 1H), 7.83-7.77 (m, 3H), 7.66-7.56 (m, 3H), 7.47 (m, 1H), 7.21 (m, 1H), 4.50 (m, 2H), 4.18 (t, J=6.2 Hz, 2H), 3.20 (t, J=5.2 Hz, 2H), 3.08 (t, J=5.9 Hz, 2H), 1.95 (m, 2H).

### Example 24

### 2-(Pyridin-2-yldisulfanyl)ethyl {3-[9-Methoxy-3-nitro-5,11-dioxo-5H-indeno[1,2-c]isoquinolin-6(11H)-yl]propyl}carbamate (72)

Compound 6 (50 mg, 0.12 mmol) was dissolved in CH₂Cl₂ (5 mL), followed by the addition of carbonate 27 (58 mg, 0.15 mmol), DMAP (15 mg, 0.12 mmol), and Et₃N (24 mg, 0.24 mmol). The mixture was stirred at room temperature for 16 h, and was then loaded directly onto a silica gel column and purified by flash column chromatography, eluting with CHCl₃, to provide the product 72 as a red solid (68.4 mg, 96%): mp 146-148° C. IR (film) 3426, 1670, 1612, 1556, 1504, 1334 cm⁻¹; ¹H NMR (300 MHz, DMSO-d₆) δ8.84 (d, J=2.2 Hz, 1H), 8.65 (d, J=8.9 Hz, 1H), 8.53 (d, J=9.1 Hz, 1H), 8.42 (s, 1H), 7.80-7.72 (m, 3H), 7.46 (m, 1H), 7.19 (m, 2H), 7.04 (d, J=8.9 Hz, 1H), 4.46 (m, 2H), 4.19 (t, J=6.4 Hz, 2H), 3.89 (s, 3H), 3.20 (t, J=5.6 Hz, 2 h), 3.09 (t, J=6.1 Hz, 2H), 1.95 (m, 2H); HPLC purity: 98.9% (MeOH, 100%).

### Example 25

### 2-(Pyridin-2-yldisulfanyl)ethyl {3-[9-Methylthio-3-nitro-5,11-dioxo-5H-indeno[1,2-c]isoquinolin-6(11H)-yl]propyl}carbamate (73)

Compound 7 (50 mg, 0.12 mmol) was dissolved in CH₂Cl₂ (5 mL), followed by the addition of carbonate 27 (56 mg, 0.14 mmol), DMAP (14 mg, 0.12 mmol), and Et₃N (23 mg, 0.23 mmol). The mixture was stirred at room temperature for 16 h, and was then loaded directly onto a silica gel column and purified by flash column chromatography, eluting with CHCl₃, to provide the product 73 as a red solid (28.8 mg, 41%): mp 152-154° C. ¹H NMR (300 MHz, DMSO-d₆) δ8.82 (s, 1H), 8.60 (d, J=9.1 Hz, 1H), 8.57 (m, 2H), 7.78 (m, 2H), 7.68 (d, J=8.1 Hz, 1H), 7.48 (m, 1H), 7.37-7.31 (m, 2H), 7.22 (m, 1H), 4.46 (m, 2H), 4.18 (m, 2H), 3.21 (m, 2H), 3.10 (m, 2H), 2.58 (s, 3H), 1.95 (m, 2H).

### Example 26

### 2-(Pyridin-2-yldisulfanyl)ethyl {3-[9-Fluoro-3-nitro-5,11-dioxo-5H-indeno[1,2-c]isoquinolin-6(11H)-yl]propyl}carbamate (74)

Compound 8 (67 mg, 0.17 mmol) was dissolved in CH₂Cl₂ (5 mL), followed by the addition of carbonate 27 (80 mg, 0.21 mmol), DMAP (20 mg, 0.17 mmol), and Et₃N (34 mg, 0.33 mmol). The mixture was stirred at room temperature for 16 h, and was then loaded directly onto a silica gel column and purified by flash column chromatography, eluting with 1% MeOH in CHCl₃, to provide the product 74 as a red solid (40.0 mg, 42%): mp 177-178° C. ¹H NMR (300 MHz, DMSO-d₆) δ8.84 (s, 1H), 8.63 (d, J=9.2 Hz, 1H), 8.52 (d, J=8.9 Hz, 1H), 8.44 (m, 1H), 7.78 (m, 1H), 7.69 (d, J=8.1 Hz, 1H), 7.49 (m, 1H), 7.31 (s, 1H), 7.22 (m, 1H), 7.16 (m, 1H), 4.43 (m, 2H), 4.18 (m, 2H), 3.23 (m, 2H), 3.08 (m, 2H), 1.94 (m, 2H); HPLC purity: 98.4% (MeOH: 100%).

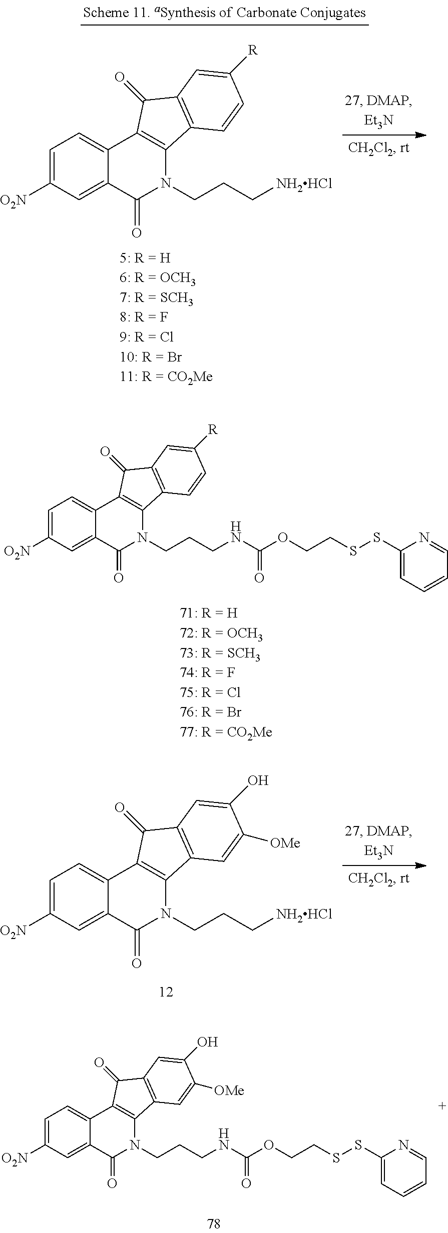

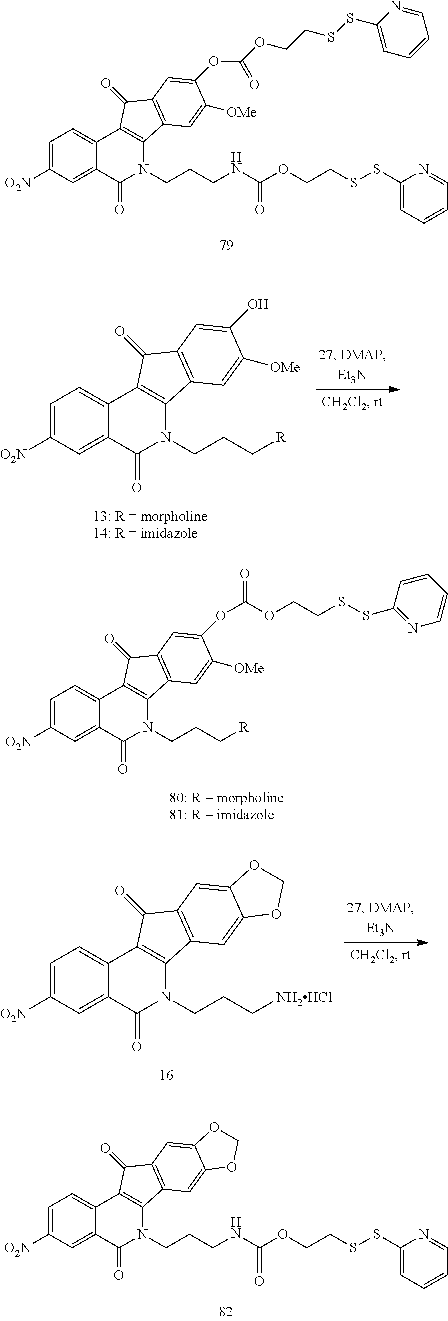

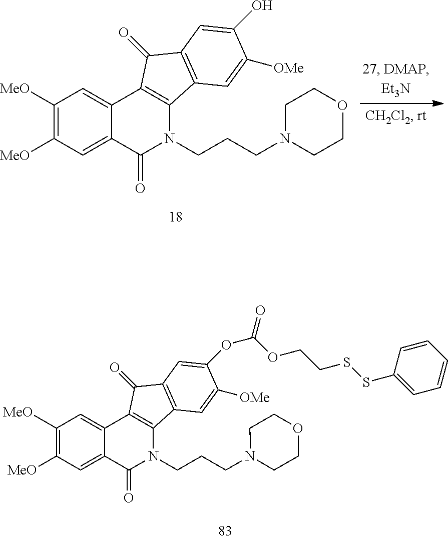 [SMILES: COc1cc2c(cc1O)C(=O)c1c-2n(CCCN2CCOCC2)c(=O)c2cc(OC)c(OC)cc12 ; COc1cc2c3c(n(CCCN4CCOCC4)c(=O)c2cc1OC)-c1cc(OC)c(OC(=O)OCCSSc2ccccc2)cc1C3=O]

### Example 27

### 2-(Pyridin-2-yldisulfanyl)ethyl {3-[9-Chloro-3-nitro-5,11-dioxo-5H-indeno[1,2-c]isoquinolin-6(11H)-yl]propyl}carbamate (75)

Compound 9 (52 mg, 0.12 mmol) was dissolved in CH₂Cl₂ (5 mL), followed by the addition of carbonate 27 (60 mg, 0.15 mmol), DMAP (15 mg, 0.12 mmol), and Et₃N (25 mg, 0.25 mmol). The mixture was stirred at room temperature for 16 h, and was then loaded directly onto a silica gel column and purified by flash column chromatography, eluting with CHCl₃, to provide the product 75 as an orange solid (38.4 mg, 52%): mp 160-161° C. IR (film) 3338, 1705, 1678, 1558, 1504, 1340, 751 cm⁻¹; ¹H NMR (300 MHz, DMSO-d₆) δ8.83 (d, J=2.1 Hz, 1H), 8.62 (d, J=9.0 Hz, 1H), 8.54 (dd, J=2.2 and 6.7 Hz, 1H), 8.41 (d, J=4.2 Hz, 1H), 7.80-7.77 (m, 3H), 7.63-7.58 (m, 2H), 7.45 (m, 1H), 7.21 (m, 1H), 4.47 (m, 2H), 4.18 (t, J=6.1 Hz, 2H), 3.20 (t, J=5.8 Hz, 2H), 3.08 (t, J=6.0 Hz, 2H), 1.94 (m, 2H); HPLC purity: 95.5% (MeOH: 100%).

### Example 28

### 2-(Pyridin-2-yldisulfanyl)ethyl {3-[9-Bromo-3-nitro-5,11-dioxo-5H-indeno[1,2-c]isoquinolin-6(11H)-yl]propyl}carbamate (76)

Compound 10 (50 mg, 0.11 mmol) was dissolved in CH₂Cl₂ (5 mL), followed by the addition of carbonate 27 (52 mg, 0.13 mmol), DMAP (13 mg, 0.11 mmol), and Et₃N (22 mg, 0.22 mmol). The mixture was stirred at room temperature for 16 h, and was then loaded directly onto a silica gel column and purified by flash column chromatography, eluting with CHCl₃, to provide the product 76 as an orange solid (46.1 mg, 67%): mp 162-164° C. ¹H NMR (300 MHz, DMSO-d₆) δ8.83 (d, J=1.9 Hz, 1H), 8.61 (d, J=8.9 Hz, 1H), 8.54 (d, J=8.6 Hz, 1H), 8.44 (m, 1H), 7.81-7.75 (m, 4H), 7.70 (d, J=5.5 Hz, 1H), 7.46 (m, 1H), 7.22 (m, 1H), 4.47 (m, 2H), 4.17 (m, 2H), 3.21 (m, 2H), 3.07 (m, 2H), 1.95 (m, 2H).

### Example 29

### Methyl 3-Nitro-5,11-dioxo-6-{3-{{[2-(pyridin-2-yldisulfanyl)ethoxy]carbonyl}amino}propyl}-6,11-dihydro-5H-indeno[1,2-c]isoquinoline-9-carboxylate (77)

Compound 11 (55 mg, 0.12 mmol) was dissolved in CH₂Cl₂ (5 mL), followed by the addition of carbonate 27 (59 mg, 0.15 mmol), DMAP (15 mg, 0.12 mmol), and Et₃N (25 mg, 0.25 mmol). The mixture was stirred at room temperature for 16 h, and was then loaded directly onto a silica gel column and purified by flash column chromatography, eluting with CHCl₃, to provide the product 77 as an orange solid (73.2 mg, 95%): mp 147-149° C. IR (film) 3404, 1720, 1670, 1603, 1518, 1338, 1252, 760 cm⁻¹; ¹H NMR (300 MHz, DMSO-d₆) δ8.83 (d, J=2.6 Hz, 1H), 8.66 (d, J=8.9 Hz, 1H), 8.55 (dd, J=2.3 and 6.6 Hz, 1H), 8.44 (m, 1H), 8.14 (d, J=7.9 Hz, 1H), 7.96 (d, J=8.2 Hz, 1H), 7.88 (s, 1H), 7.81 (m, 2H), 7.50 (m, 1H), 7.22 (m, 1H), 4.51 (m, 2H), 4.20 (t, J=6.2 Hz, 2H), 3.89 (s, 3H), 3.25 (m, 2H), 3.13 (m, 2H), 1.98 (m, 2H); HPLC purity: 96.1% (MeOH: 100%).

### Example 30

### 2-(Pyridin-2-yldisulfanyl)ethyl {3-{8-Methoxy-3-nitro-5,11-dioxo-9-{{[2-(pyridin-2-yldisulfanyl)ethoxy]carbonyl}oxy}-5H-indeno[1,2-c]isoquinolin-6(11H)-yl}propyl}carbamate (78) and 2-(Pyridin-2-yldisulfanyl)ethyl (3-(9-hydroxy-8-methoxy-3-nitro-5,11-dioxo-5H-indeno[1,2-c]isoquinolin-6(11H)-yl)propyl)carbamate (79)

Compound 12 (8.7 mg, 0.018 mmol) was diluted in CH₂Cl₂ (20 mL), followed by the addition of carbonate 27 (7.0 mg, 0.018 mmol), DMAP (2.3 mg, 0.018 mmol), and Et₃N (3.7 mg, 0.036 mmol). The mixture was stirred at room temperature for 16 h, during which all the material dissolved completely to give a clear red solution. The solution was then loaded directly onto a silica gel column and purified by flash column chromatography, eluting with 2%-4% MeOH in CHCl₃, to provide both products.

Carbamate 78 (8.2 mg, 72%): ¹H NMR (300 MHz, DMSO-d₆) δ10.36 (s, 1H), 8.81 (d, J=2.3 Hz, 1H), 8.56 (d, J=9.1 Hz, 1H), 8.47-8.41 (m, 2H), 7.80-7.74 (m, 2H), 7.45 (t, J=5.8 Hz, 1H), 7.22-7.17 (m, 2H), 6.99 (s, 1H), 4.46 (m, 2H), 4.18 (t, J=6.1 Hz, 2H), 3.96 (s, 3H), 3.22 (m, 2H), 3.07 (t, J=5.9 Hz, 2H), 1.96 (m, 2H).

Dicarbonate 79 (4.3 mg, 28%): mp 115-117° C. IR (film) 3292, 1766, 1705, 1674, 1614, 1560, 1507, 1418, 1338, 1252, 760 cm⁻¹; ¹H NMR (300 MHz, DMSO-d₆) δ8.88 (d, J=2.4 Hz, 1H), 8.69 (d, J=8.9 Hz, 1H), 8.56 (dd, J=2.4 and 6.6 Hz, 1H), 8.47 (d, J=4.6 Hz, 1H), 8.42 (d, J=4.4 Hz, 1H), 7.86-7.73 (m, 4H), 7.58 (s, 1H), 7.42 (m, 2H), 7.27 (m, 1H), 7.21 (m, 1H), 4.54 (m, 2H), 4.48 (t, J=6.1 Hz, 2H), 4.16 (t, J=6.3 Hz, 2H), 4.05 (s, 3H), 3.24 (m, 4H), 3.06 (t, J=6.2 Hz, 2H), 1.99 (m, 2H); ESIMS m/z (rel. intensity) 844 (MNa⁺, 100); HRMSESI calcd for MH⁺: 822.1032. found: 822.1036.

### Example 31

### 8-Methoxy-6-(3-morpholinopropyl)-3-nitro-5,11-dioxo-6,11-dihydro-5H-indeno[1,2-c]isoquinolin-9-yl[2-(pyridin-2-yldisulfanyl)ethyl] Carbonate (80)

Compound 13 (20 mg, 0.037 mmol) was diluted in CH₂Cl₂ (20 mL), followed by the addition of carbonate 27 (17 mg, 0.044 mmol), DMAP (4.5 mg, 0.037 mmol), and Et₃N (9.3 mg, 0.092 mmol). The mixture was stirred at room temperature for 16 h, during which all the material dissolved completely to give a clear orange solution. The solution was then loaded directly onto a silica gel column and purified by flash column chromatography, eluting with 0%-2% MeOH in CHCl₃, to provide the product 80 as an orange solid (12.4 mg, 50%): mp 133-135° C. IR (film) 1764, 1675, 1613, 1560, 1508, 1337, 1285, 1251, 1190 cm⁻¹; ¹H NMR (300 MHz, DMSO-d₆) δ8.89 (d, J=2.6 Hz, 1H), 8.71 (d, J=8.8 Hz, 1H), 8.58 (dd, J=2.2 and 6.6 Hz, 1H), 8.47 (d, J−=4.8 Hz, 1H), 7.84 (m, 2H), 7.59 (s, 1H), 7.47 (s, 1H), 7.26 (s, 1H), 4.64 (m, 2H), 4.46 (t, J=6.0 Hz, 2H), 4.04 (s, 3H), 3.39 (m, 6H), 3.24 (t, J=5.5 Hz, 2H), 2.29 (m, 4H), 2.02 (m, 2H); APCI-MS m/z (rel. intensity) 679 (MH⁺, 100); HRMS (+ESI) calcd for MH⁺: 679.1533. found: 679.1528; HPLC purity: 100% (MeOH: 100%).

### Example 32

### 6-[3-(1H-Imidazol-1-yl)propyl]-8-methoxy-3-nitro-5,11-dioxo-6,11-dihydro-5H-indeno[1,2-c]isoquinolin-9-yl[2-(pyridin-2-yldisulfanyl)ethyl] Carbonate (81)

Compound 14 (20 mg, 0.038 mmol) was diluted in CH₂Cl₂ (20 mL), followed by the addition of carbonate 27 (18 mg, 0.046 mmol), DMAP (4.6 mg, 0.038 mmol), and Et₃N (10 mg, 0.095 mmol). The mixture was stirred at room temperature for 16 h, during which all the material dissolved completely to give a clear orange solution. The solution was then loaded directly onto a silica gel column and purified by flash column chromatography, eluting with 0%-3% MeOH in CHCl₃, to provide the product 81 as an orange solid (9.4 mg, 38%): mp 168-170° C. (dec). IR (film) 1756, 1669, 1611, 1556, 1503, 1423, 1335, 1187 cm⁻¹; ¹H NMR (300 MHz, DMSO-d₆) δ8.80 (s, 1H), 8.56 (m, 1H), 8.46 (m 2H), 8.16 (m, 1H), 7.84-7.77 (m, 2H), 7.52 (m, 2H), 7.29 (m, 2H), 7.08 (m, 1H), 4.55 (m, 4H), 4.45 (m, 2H), 4.27 (s, 3H), 3.22 (m, 2H), 2.32 (m, 2H); ESIMS m/z (rel. intensity) 694/696 (MCI, 100); HRMSESI calcd for MCI: 694.0833. found: 694.0840; HPLC purity: 100% (MeOH: 100%), 97.7 (MeOH—H₂O, 90:10).

### Example 33

### 2-(Pyridin-2-yldisulfanyl)ethyl {3-[8,9-Methylenedioxy-3-nitro-5,11-dioxo-5H-indeno[1,2-c]isoquinolin-6(11H)-yl]propyl}carbamate (82)

Compound 16 (50 mg, 0.12 mmol) was dissolved in CH₂Cl₂ (5 mL), followed by the addition of carbonate 27 (54 mg, 0.14 mmol), DMAP (14 mg, 0.12 mmol), and Et₃N (118 mg, 1.2 mmol). The mixture was stirred at room temperature for 16 h, and was then loaded directly onto a silica gel column and purified by flash column chromatography, eluting with CHCl₃, to provide the product 82 as a brown solid (38.9 mg, 55%): mp 167-169° C. IR (film) 3435, 1699, 1677, 1611, 1553, 1500, 1332, 1308, 1290, 1028 cm⁻¹; ¹H NMR (300 MHz, DMSO-d₆) δ8.76 (s, 1H), 8.51-8.43 (m, 3H), 7.80-7.77 (m, 2H), 7.44 (m, 1H), 7.31 (s, 1H), 7.21 (m, 1H), 7.14 (s, 1H), 6.21 (s, 2H), 4.40 (m, 2H), 4.18 (t, J=6.3 Hz, 2H), 3.17 (t, J=5.4 Hz, 2H), 3.07 (t, J=6.0 Hz, 2H), 1.91 (m, 2H).

### Example 34

### 2-(pyridin-2-yldisulfanyl)ethyl {2,3,8-Trimethoxy-6-(3-morpholinopropyl)-5,11-dioxo-6,11-dihydro-511-indeno[1,2-c]isoquinolin-9-yl}Carbonate (83)

Compound 18 (50 mg, 0.10 mmol) was dissolved in CH₂Cl₂ (5 mL), followed by the addition of carbonate 27 (48 mg, 0.12 mmol), DMAP (13 mg, 0.10 mmol), and Et₃N (21 mg, 0.21 mmol). The mixture was stirred at room temperature for 16 h, and was then loaded directly onto a silica gel column and purified by flash column chromatography, eluting with 3% MeOH in CHCl₃, to provide the product 83 as a red solid (64.8 mg, 90%): mp 130-132° C. ¹H NMR (300 MHz, CDCl₃) δ8.51 (d, J=4.5 Hz, 1H), 8.09 (s, 1H), 7.69-7.66 (m, 3H), 7.37 (s, 1H), 7.16-7.12 (m, 2H), 4.59 (q, J=6.6 Hz, 4H), 4.06 (s, 3H), 4.00 (s, 3H), 3.98 (s, 3H), 3.68 (t, J=4.3 Hz, 4H), 3.18 (t, J=6.5 Hz, 2H), 2.59 (t, J=6.9 Hz, 2H), 2.47 (br s, 4H), 2.14 (m, 2H).

### Example 35

### 2-(Pyridin-2-yldisulfanyl)ethanol Hydrochloride (36)

2-Mercaptoethanol (33) (0.77 g, 9.9 mmol) was dissolved in CH₃CN (5 mL) and added dropwise to a solution of methoxycarbonylsulfenyl chloride (34) (1.25 g, 9.9 mmol) in CH₃CN (8 mL) precooled at 0° C. The pale yellow solution was stirred at 0° C. for 30 min until it turned colorless. A solution of 2-mercaptopyridine (35) (1.0 g, 9.0 mmol) in CH₃CN (20 mL) was added dropwise to the clear solution, and the yellow mixture was stirred at reflux for 2 h, during which a white precipitate formed. The colorless mixture with white precipitate was then stirred at 0° C. for 1 h and filtered. The filter cake was washed with CH₃CN to provide the product 36 as a white amorphous solid (1.84 g, 92%): mp 128-130° C. ¹H NMR (300 MHz, CDCl₃) δ9.13 (d, J=5.5 Hz, 1H), 8.10 (t, J=7.4 Hz, 1H), 7.82 (d, J=8.3 Hz, 1H), 7.61 (t, J=6.6 Hz, 1H), 4.01 (t, J=5.2 Hz, 2H), 3.27 (t, J=5.6 Hz, 2H); ESIMS (positive mode) m/z (rel. intensity) 188 [(MH⁺—H₂O)⁺, 100].

### Example 36

### 1H-Benzo[d][1,2,3]triazol-1-yl[2-(pyridin-2-yldisulfanyl)ethyl] Carbonate Hydrochloride (27)

Compound 36 (1.00 g, 4.47 mmol) was dissolved in CH₂Cl₂ (5 mL) and Et₃N (0.45 g, 4.47 mmol) and added dropwise to a solution of triphosgene (37) (0.44 g, 1.49 mmol) at 0° C. The solution was stirred at room temperature for 1.5 h, followed by a dropwise addition of a solution of hydroxybenzotriazole (38) (0.60 g, 4.47 mmol) in CH₂Cl₂ (10 mL) and Et₃N (0.45 g, 4.47 mmol). The mixture was then stirred at room temperature for 16 h, and then diluted with CHCl₃ to 50 mL, washed with H₂O (100 mL×3) and brine (100 mL). The organic layer was dried over anhydrous Na₂SO₄, filtered and concentrated. The resultant yellow oil was triturated with hexane and filtered to provide the product 27 as a white solid (1.36 g, 79%): mp 116-118° C. ¹H NMR (300 MHz, CDCl₃) δ8.40 (d, J=4.8 Hz, 1H), 8.18 (d, J=8.4 Hz, 1H), 8.04 (d, J=8.4 Hz, 1H), 7.92 (t, J=8.0 Hz, 1H), 7.77-7.74 (m, 2H), 7.66 (t, J=7.8 Hz, 1H), 7.19 (m, 1H), 4.74 (t, J=6.0 Hz, 2H), 3.38 (t, J=6.0 Hz, 2H).

### Example 37

### 2-(Pyridin-2-yldisulfanyl)ethyl Hydrazinecarboxylate (49)

Carbonate 27 (500 mg, 1.30 mmol), DIPEA (334 mg, 2.60 mmol), and N₂H₄.H₂O (130 mg, 2.60 mmol) were diluted in CH₂Cl₂ (5 mL), and the mixture was stirred at 0° C. for 1.5 h. The yellow solution was then diluted to 30 mL with CHCl₃, and washed with H₂O (100 mL×3), and brine (100 mL). The organic layer was dried over anhydrous Na₂SO₄, filtered and concentrated to provide the product 49 as a yellow liquid. ¹H NMR (300 MHz, CDCl₃) δ8.49 (d, J=4.9 Hz, 1H), 7.67-7.61 (m, 2H), 7.13 (m, 1H), 4.41 (dd, J=2.5 and 3.9 Hz, 2H), 3.73 (s, 1H), 3.09 (t, J=6.4 Hz, 2H)

### Example 38

### 5-Benzyl 1-(tert-Butyl) (((S)-1,5-di-tert-butoxy-1,5-dioxopentan-2-yl)carbamoyl)-L-glutamate (43)

(Kularatne, et al., Mol. Pharm. 2009, 6, 790-800)

L-Glu(O^{t}Bu)-O^{t}Bu (40) (500 mg, 1.69 mmol) and triphosgene (168 mg, 0.565 mmol) were diluted in CH₂Cl₂ (25 mL) at 0° C. in argon for 5 min, and then Et₃N (376 mg, 3.72 mmol) was added. The mixture was stirred at 0° C. for 2 h, followed by an addition of L-Glu(OBn)-O^{t}Bu (42) (613 mg, 1.86 mmol) in Et₃N (244 mg, 2.42 mmol) and CH₂Cl₂ (5 mL). Stirring was continued at room temperature for 16 h, and then the reaction was quenched with 1 M HCl (50 mL). The organic layer was concentrated to a yellow syrup, which was purified with flash column chromatography, eluting with a 30%-50% gradient of EtOAc in hexane to provide the urea 43 as a clear colorless syrup (0.94 g, 96%). ¹H NMR (300 MHz, CDCl₃) δ7.34 (s, 5H), 5.11 (s, 2H), 5.05-5.00 (m, 2H), 4.40-4.31 (m, 2H), 2.49-2.40 (m, 2H), 2.37-2.26 (m, 2H), 2.22-2.05 (m, 2H), 1.96-1.82 (m, 2H), 1.46-1.43 (s, 27H).

### Example 39

### (S)-5-(tert-Butoxy)-4-(3-(((S)-1,5-di-tert-butoxy-1,5-dioxopentan-2-yl)ureido)-5-oxopentanoic Acid (44)

(Kularatne, et al., Mol. Pharm. 2009, 6, 790-800)

Compound 43 (0.96 g, 1.66 mmol) was diluted in EtOAc (15 mL), and the mixture was degassed in 5 min with argon, followed by an addition of 10% Pd on activated charcoal (20 mg), and the mixture was degassed for another 5 min. The mixture was hydrogenated by room temperature with a hydrogen balloon for 16 h, and was then filtered and washed with EtOAc through a Celite pad. The solution was concentrated and purified with flash column chromatography (SiO₂), eluting with a 30%-50% gradient of EtOAc in hexane to provide a clear colorless syrup. The syrup was triturated with hexane and let stand overnight to yield the DUPA precursor 44 as a white semisolid (0.70 g, 86%). ¹H NMR (300 MHz, CDCl₃) δ5.84 (d, J=8.2 Hz, 1H), 5.42 (br s, 1H), 4.44 (m, 1H), 4.34 (m, 1H), 2.43-2.39 (m, 2H), 2.36-2.28 (m, 2H), 2.24-2.03 (m, 2H), 1.91-1.79 (m, 2H), 1.48 (s, 9H), 1.46 (s, 9H), 1.44 (s, 9H). Note: all of the following melting points (noted with an asterisk *) are defined as the temperature at which the solid sample started to soften into a semi-liquid, sticky gum that never reached the liquid phase.

### Example 40

### Fmoc-Solid Phase Peptide Synthesis of DUPA-Aoc-Phe-Phe-Dap-Asp-Cys Reagent (28)

(Kularatne, et al., Mol. Pharm. 2009, 6, 790-800)

H-L-Cys(Trt)-(2-ClTrt) resin (45) (0.7 meq/g, 200 mg, 0.14 mmol) was swollen in CH₂Cl₂ (5 mL) for 30 min while the mixture was being bubbled with argon. CH₂Cl₂ was drained, and a solution of Fmoc-L-Asp(O^{t}Bu)-OH (2.5 eq), PyBOP (2.5 eq), HOBt (2.5 eq), and DIPEA (5.0 eq) in DMF (3 mL) was added to the resin. The mixture was bubbled with argon in 3 h, and then drained. The resin was washed with DMF (5 mL×3, in 5 min/wash, drained after each wash) and PrOH (5 mL×3, in 5 min/wash, drained after each wash). A Kaiser test was performed to give a negative result, which indicated the coupling reaction was successful. The resin was then washed with 20% piperidine in DMF (5 mL×3, in 10 min/wash, drained after each wash), DMF (5 mL×3, in 5 min/wash, drained after each wash) and i-PrOH (5 mL×3, in 5 min/wash, drained after each wash).

A second Kaiser test was performed to give a positive result, which indicated the to cleavage of Fmoc group was successful. The above sequence was repeated for the coupling of Boc-L-Dap(Fmoc)-OH, Fmoc-L-Phe-OH, Fmoc-L-Phe-OH, Fmoc-8-Aoc-OH, and the protected DUPA precursor. The final product was cleaved from the resin by washing with a TFA:H₂O:TIPS:1,2-ethanedithiol cocktail (92.5:2.5:2.5:2.5) (7.5 mL, 30 min) during which argon was bubbled. Another 7.5-mL portion of the cocktail was diluted with TFA (7.5 mL) to make a 15-mL solution. This solution was used to wash the resin twice (7.5 mL/wash, in 15 min/wash). The filtrate was collected and concentrated. The resulting syrup was precipitated in Et₂O; the mixture was centrifuged, and the precipitate was collected. The crude product was purified with preparative RP-HPLC [λ=254 nm; solvent gradient: 0% B to 80% B in 30 min; A=aqueous NH₄OAc/AcOH buffer at pH=5; B=MeCN]. Pure fractions were combined, concentrated under vacuum, and lyophilized in 48 h to yield the pure DUPA-peptide product 28 as a white solid (172 mg, 58% overall yield, or 91.3% average yield per coupling step): mp* 175-178° C. ¹H NMR (300 MHz, DMSO-d₆) δ9.39 (d, J=9.10 Hz, 1H), 8.92 (d, J=8.2 Hz, 1H), 8.68 (m, 1H), 8.16 (m, 1H), 7.81 (m, 1H), 7.71 (d, J=5.6 Hz, 1H), 7.29-7.1, 10H), 6.45 (m, 1H), 6.36 (m, 1H), 4.43 (m, 4H), 4.22 (q, J=6.6 Hz, 3H), 4.03-3.96 (m, 6H), 3.43-3.36 (m, 2H), 3.14-2.84 (m, 7H), 2.63 (d, J=6.6 Hz, 3H), 2.20-2.18 (m, 2H), 2.07 (m, 2H), 2.02-1.94 (m, 1H), 1.91-1.80 (m, 3H), 1.74-1.68 (m, 3H), 1.31-1.26 (m, 4H), 1.17-1.03 (m, 8H); LC/MS (ES-API) m/z 1060.2 (M⁺), and 530.7 (M²⁺). UV/vis: λₘₐₓ=254 nm

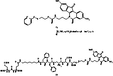 [SMILES: COc1ccc2c(c1)C(=O)c1c-2n(CCCNC(=O)OCCSSc2ccccn2)c(=O)c2cc([N+](=O)[O-])ccc12 ; COc1ccc2c(c1)C(=O)c1c-2n(CCCNC(=O)OCCSSC[C@H](NC(=O)[C@H](CC(=O)O)NC(=O)[C@@H](N)CNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)C(=O)O)c(=O)c2cc([N+](=O)[O-])ccc12]

### Example 41

### (12R,15S,18S,22S,25S,39S,43S)-18-Amino-22,25-dibenzyl-15-(carboxymethyl)-1-(9-methoxy-3-nitro-5,11-dioxo-5,11-dihydro-6H-indeno[1,2-c]isoquinolin-6-yl)-5,14,17,21,24,27,36,41-octaoxo-6-oxa-9,10-dithia-4,13,16,20,23,26,35,40,42-nonaazapentatetracontane-12,39,43,45-tetracarboxylic acid (84)

DUPA-peptide 28 (35.8 mg, 0.034 mmol) was dissolved in an aqueous buffer solution of NH₄OAc (2 mL) at pH=6, followed by an addition of carbonate 72 (20.0 mg, 0.034 mmol) in THF (4 mL). The mixture was stirred at room temperature for 1 h, and then concentrated under vacuum. The concentrate was purified with preparative RP-HPLC [λ=254 nm; solvent gradient: 0% B to 80% B in 30 min; A=aqueous NH₄OAc/AcOH buffer at pH=7; B=MeCN] to provide the desired product 84 as an orange solid (15.5 mg, 29.8%): mp* 215-217° C. ¹H NMR (500 MHz, DMSO-d₆+1 drop of D₂O): δ8.82 (s, 1H), 8.61 (d, J=8.6 Hz, 1H), 8.49 (d, J=9.1 Hz, 1H), 7.72 (d, J=8.1 Hz, 1H), 7.22-7.04 (m, 12H), 4.42-4.37 (m, 4H), 4.17 (m, 1H), 4.11 (m, 3H), 3.89-3.84 (m, 5H), 3.42-3.26 (m, 2H), 3.15-3.11 (m, 2H), 3.03-2.79 (m, 7H), 2.57-2.50 (m, 5H), 2.16 (m, 2H), 2.03 (m, 2H), 1.91-1.77 (m, 6H), 1.73-1.66 (m, 3H), 1.24 (m, 5H), 1.06 (m, 4H), 0.95 (m, 2H); MALDI-MS (rel intensity) m/z 1541 (MH⁺); HRMS (+ESI) calcd for MH⁺: 1541.5241. found 1541.5233 (Δm/m=0.5 ppm); UV/vis: λₘₐₓ=254 nm.

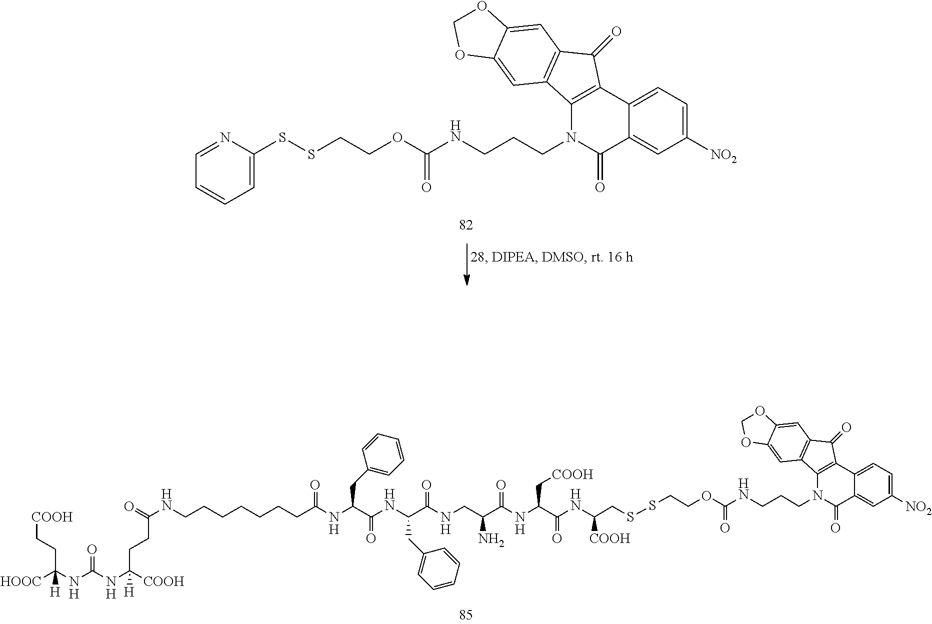 [SMILES: O=C(NCCCn1c2c(c3ccc([N+](=O)[O-])cc3c1=O)C(=O)c1cc3c(cc1-2)OCO3)OCCSSc1ccccn1 ; N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CSSCCOC(=O)NCCCn1c2c(c3ccc([N+](=O)[O-])cc3c1=O)C(=O)c1cc3c(cc1-2)OCO3)C(=O)O]

### Example 42

### (12R,15S,18S,22S,25S,39S,43S)-18-Amino-22,25-dibenzyl-15-(carboxymethyl)-1-(3-nitro-5,12-dioxo-5,12-dihydro-6H-[1,3]dioxolo[4′,5′:5,6]indeno[1,2-c]isoquinolin-6-yl)-5,14,17,21,24,27,36,41-octaoxo-6-oxa-9,10-dithia-4,13,16,20,23,26,35,40,42-nonaazapentatetracontane-12,39,43,45-tetracarboxylic Acid (85)

DUPA-peptide 28 (35.0 mg, 0.033 mmol) and carbonate 82 (20.0 mg, 0.033 mmol) were dissolved in DMSO (3 mL) and DIPEA (8.5 mg, 0.066 mmol). The mixture was stirred at room temperature for 16 h, and then purified with preparative RP-HPLC [λ=254 nm; solvent gradient: 0% B to 80% B in 30 min; A=aqueous NH₄OAc/AcOH buffer at pH=7; B=MeCN] to provide the desired product 85 as a brown solid (31.5 mg, 61.4%): mp* 190-192° C. ¹H NMR (500 MHz, DMSO-d₆+1 drop of D₂O): δ8.75 (s, 1H), 8.50 (d, J=9.2 Hz, 1H), 8.42 (d, J=10.8 Hz, 1H), 7.27 (s, 1H), 7.20-7.07 (m, 11H), 6.18 (s, 2H), 4.58 (m, 1H), 4.44-4.38 (m, 4H), 4.20 (d, J=6.5 Hz, 1H), 4.12 (m, 2H), 3.92 (m, 2H), 3.44 (m, 1H), 3.27 (d, J=9.0 Hz, 1H), 2.98-2.90 (m, 3H), 2.88-2.76 (m, 5H), 2.60-2.52 (m, 5H), 2.19 (t, J=7.8 Hz, 1H), 2.04 (m, 1H), 2.86-1.64 (m, 7H), 1.25 (m, 5H), 1.07 (m, 4H), 0.95 (m, 2H); UV/vis: λₘₐₓ=254 nm.

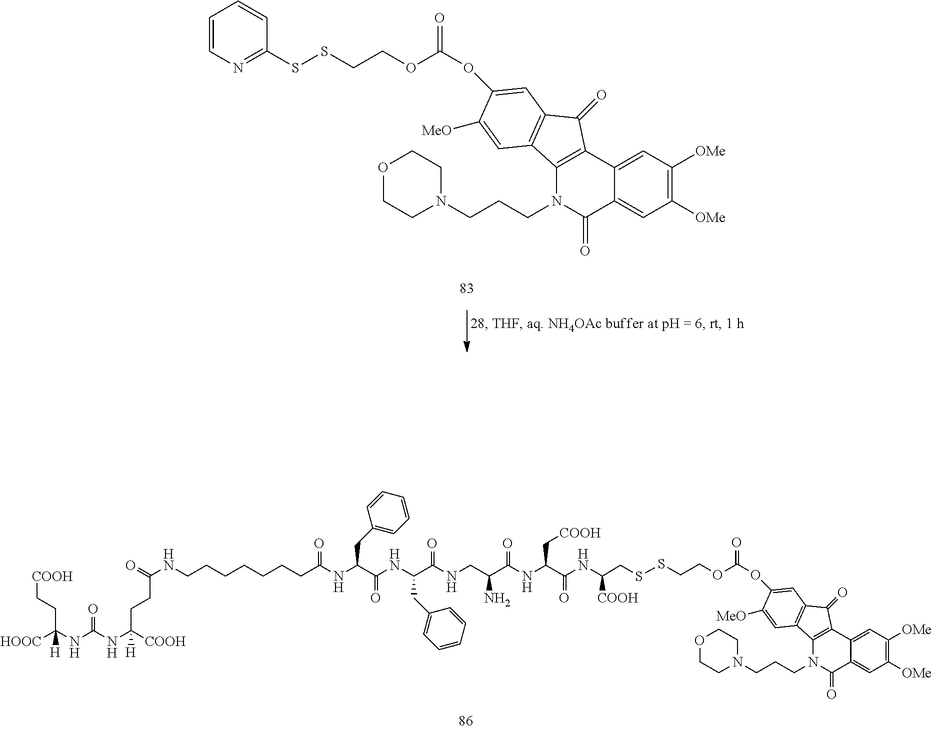 [SMILES: COc1cc2c3c(n(CCCN4CCOCC4)c(=O)c2cc1OC)-c1cc(OC)c(OC(=O)OCCSSc2ccccn2)cc1C3=O ; COc1cc2c3c(n(CCCN4CCOCC4)c(=O)c2cc1OC)-c1cc(OC)c(OC(=O)OCCSSC[C@H](NC(=O)[C@H](CC(=O)O)NC(=O)[C@@H](N)CNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)C(=O)O)cc1C3=O]

### Example 43

### (8R,11S,14S,18S,21S,35S,39S)-14-Amino-18,21-dibenzyl-11-(carboxymethyl)-1,10,13,17,20,23,32,37-octaoxo-1-((2,3,8-trimethoxy-6-(3-morpholinopropyl)-5,11-dioxo-6,11-dihydro-5H-indeno[1,2-c]isoquinolin-9-yl)oxy)-2-oxa-5,6-dithia-9,12,16,19,22,31,36,38-octaazahentetracontane-8,35,39,41-tetracarboxylic Acid (86)

Method A: DUPA-peptide 28 (30.6 mg, 0.028 mmol) was dissolved in an aqueous buffer solution of NH₄OAc (2 mL) at pH=6, followed by an addition of carbonate 83 (20.0 mg, 0.028 mmol) in THF (2 mL). The mixture was stirred at room temperature for 1 h, and then concentrated under vacuum. The concentrate was purified with preparative RP-HPLC [λ=254 nm; solvent gradient: 0% B to 80% B in 30 min; A=aqueous NH₄OAc/AcOH buffer at pH=7; B=MeCN] to provide the desired product 86 as a plum-colored solid (29.6 mg, 62.5%): mp* 188-190° C. ¹H NMR (500 MHz, DMSO-d₆+1 drop of D₂O): δ7.88 (s, 1H), 7.48 (s, 1H), 7.36 (s, 1H), 7.22-7.06 (m, 11H), 4.50-4.48 (m, 2H), 4.40-4.35 (m, 5H), 4.20 (m, 1H), 4.09 (t, J=5.3 Hz, 1H), 3.83 (s, 3H), 3.91-3.86 (m, 8H), 3.83-3.81 (m, 3H), 3.78 (s, 1H), 3.40 (m, 7H), 3.26 (m, 1H), 3.13-3.07 (m, 2H), 2.98 (m, 3H), 2.93 (m, 2H), 2.83 (m, 1H), 2.64-2.46 (m, 3H), 2.43 (t, J=6.4 Hz, 1H), 2.27 (m, 4H), 2.18 (t, J=7.3 Hz, 2H), 2.04 (m, 2H), 1.95-1.85 (m, 8H), 1.73 (m, 2H), 1.26 (m, 5H), 1.07 (m, 5H), 0.96 (m, 2H); MALDI-MS (rel intensity) m/z 1642 (MH⁺); HRMS (+ESI) calcd for MH⁺: 1642.5969. found 1642.6043 (Δm/m=4.5 ppm); UV/vis: λₘₐₓ=254 nm.

Method B:

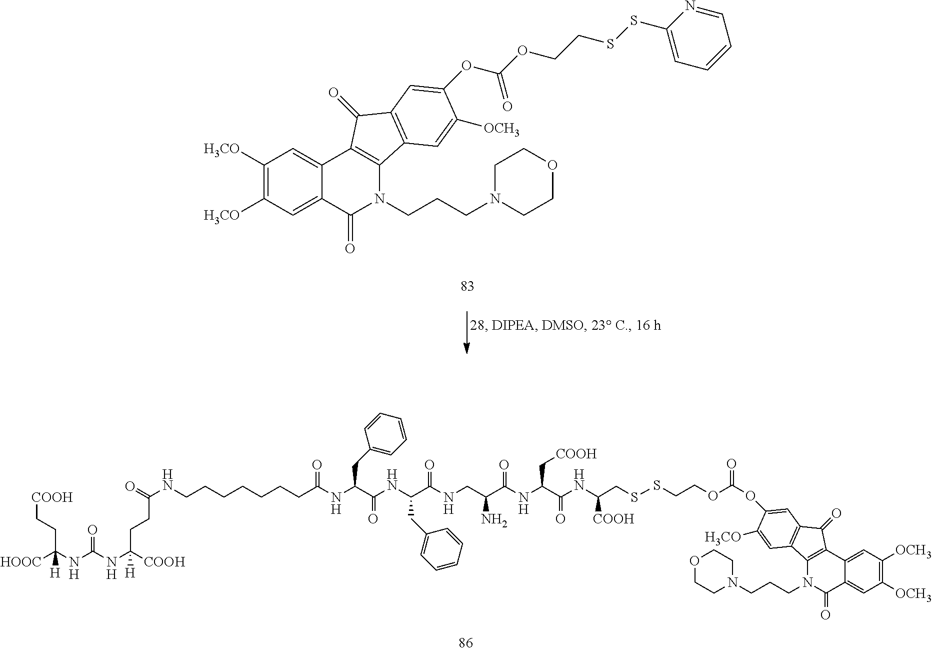 [SMILES: COc1cc2c3c(n(CCCN4CCOCC4)c(=O)c2cc1OC)-c1cc(OC)c(OC(=O)OCCSSc2ccccn2)cc1C3=O ; COc1cc2c3c(n(CCCN4CCOCC4)c(=O)c2cc1OC)-c1cc(OC)c(OC(=O)OCCSSC[C@H](NC(=O)[C@H](CC(=O)O)NC(=O)[C@@H](N)CNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)C(=O)O)cc1C3=O]

Carbonate 83 (15 mg, 0.022 mmol) and DUPA-peptide reagent 28 (23 mg, 0.022 mmol) were dissolved in DMSO (3 mL) and DIPEA (5.6 mg, 0.043 mmol). The mixture was stirred at room temperature for 16 h and then purified by preparative RP-HPLC [λ=280 nm; solvent gradient: 0% B to 80% B in 30 min; A=aqueous NH₄OAc/AcOH buffer at pH=7; B=MeCN] to provide the product 86 as a plum-colored solid (22.3 mg, 63%). ¹H NMR (500 MHz, DMSO-d₆+1 drop of D₂O): δ7.88 (s, 1H), 7.48 (s, 1H), 7.36 (s, 1H), 7.22-7.06 (m, 11H), 4.50-4.48 (m, 2H), 4.40-4.35 (m, 5H), 4.20 (m, 1H), 4.09 (t, J=5.3 Hz, 1H), 3.83 (s, 3H), 3.91-3.86 (m, 8H), 3.83-3.81 (m, 3H), 3.78 (s, 1H), 3.40 (m, 7H), 3.26 (m, 1H), 3.13-3.07 (m, 2H), 2.98 (m, 3H), 2.93 (m, 2H), 2.83 (m, 1H), 2.64-2.46 (m, 3H), 2.43 (t, J=6.4 Hz, 1H), 2.27 (m, 4H), 2.18 (t, J=7.3 Hz, 2H), 2.04 (m, 2H), 1.95-1.85 (m, 8H), 1.73 (m, 2H), 1.26 (m, 5H), 1.07 (m, 5H), 0.96 (m, 2H); MALDI-MS (rel intensity) m/z 1642 (MH⁺); HRMS (+ESI) calcd for MH⁺ (C₇₆H₉₆N₁₁O₂₆S₂): 1642.5969. found 1642.6043 (Δm/m=4.5 ppm); UV/vis: λₘₐₓ=280 nm; HPLC purity: 97.2% (MeCN, 100%).

### Biological Data

### Example 44

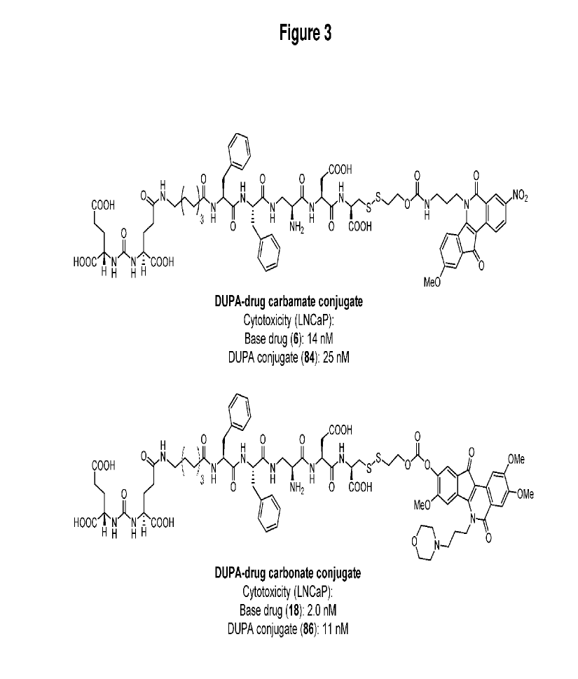

As an example to illustrate the promising effectiveness of this method, the cytotoxicities of the base drugs 6 and 18, and their corresponding DUPA conjugates 84 and 86 in LNCap cell cultures were determined to be in low nanomolar range (FIG. 3). The excellent cytotoxicities of the DUPA conjugates served as an indication that the disulfide reduction and conversion of the intermediates to the base drugs (6 and 18) are occurring intracellularly. Although an increase in potency has not been observed yet, this encouraging result partially supported our initial hypothesis, and urged further optimization of the peptide linker to facilitate the drug releasing mechanism.

### Example 45

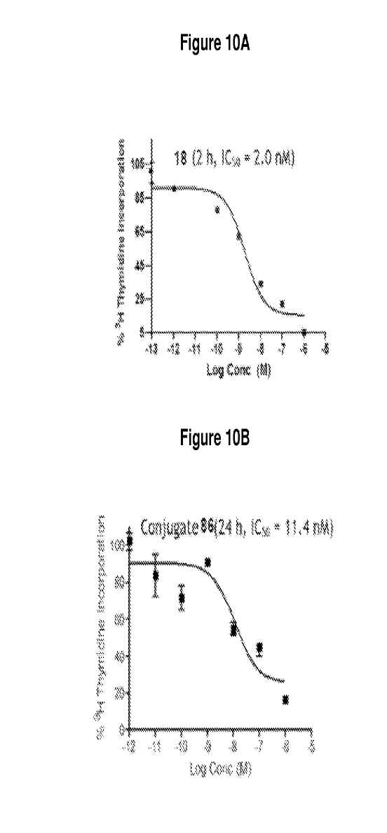

The cytotoxicities of the free drug 18 and its DUPA conjugate 86 were evaluated in 22RV1 cell culture and the IC₅₀ values were quantified in dose-dependent ³H-thymidine incorporation assays to be in the low nanomolar range (representative graphs are depicted in FIG. 10). The IC₅₀ value of the indenoisoquinoline 18 itself was 2.0 nM when determined after a 2 h incubation. The conjugate 86 showed no activity after a 2-hour incubation, but it produced an IC₅₀ value of 11.4 nM after a 24-hour incubation. An increase in potency of 86 relative to 18 was not observed. In fact, the conjugate 86 was slightly less potent than the drug 18 itself. However, the potential value of the conjugate 86 is lack of cytotoxicity in “normal” cells, which would result in 86 being a less toxic anticancer drug.

### Example 46

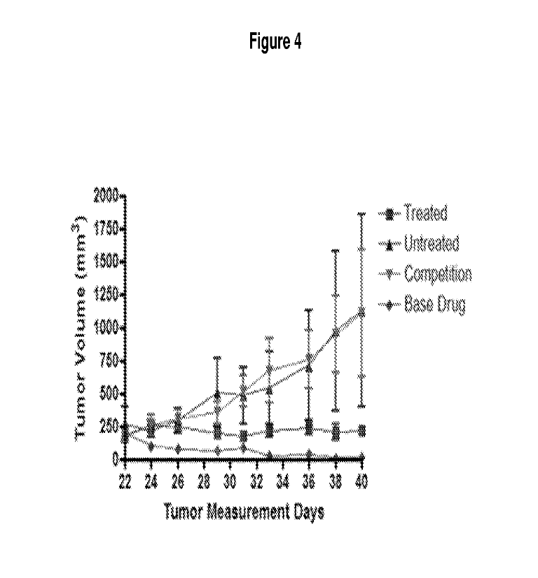

In order to demonstrate the efficacy and investigate toxicity in an animal model, 22RV1 xenograft-bearing mice (similar to LNCap xenograft model) were treated with the indenoisoquinoline 18 and its DUPA conjugate 86 at a dose of 40 nmol/mouse (2.0 μmol/kg) by IP injection with a single dose on alternate days, 3 days/week for 3 weeks (9 doses in total) (FIGS. 4-6). Four groups of mice were utilized in the experiment: (a) the untreated group (▴) served as the control, (b) the free-drug group (♦) was treated with the free drug 18, (c) the treated group (▪) was given the DUPA conjugate 86, and (d) the competitor group (▾) received both the DUPA conjugate 86 and the DUPA-peptide reagent 28, whose concentration was in 10-fold excess of 86. The reagent 28 served as a PSMA competitor and was used in a much higher concentration in order to completely saturate all PSMA available for DUPA-binding, thus preventing the PSMA-mediated uptake of the DUPA conjugate 86 if the uptake of 86 is in fact PSMA-mediated.

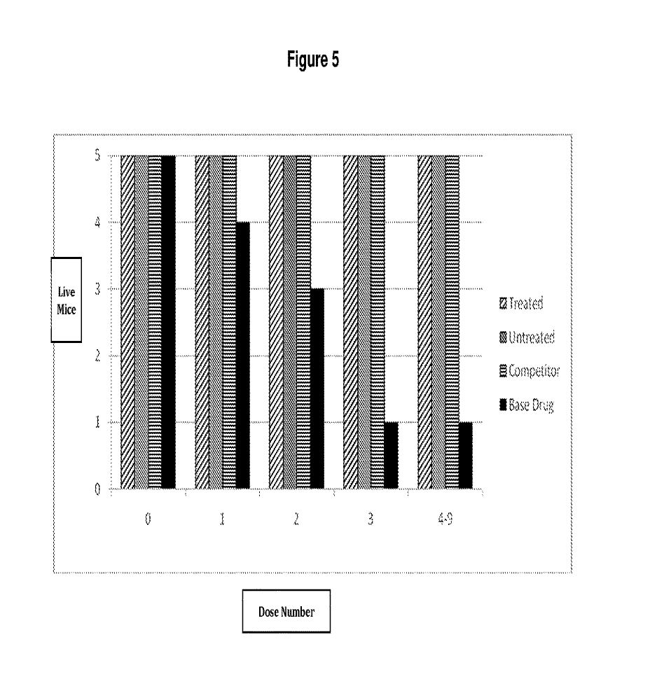

The result in FIG. 4 shows a complete cessation and regression of tumor growth during the treatment period for the DUPA-treated and base drug-treated groups, respectively, as compared to the untreated group or group treated with the DUPA conjugate 86 and the PSMA competitor 28, which implied that the uptake of DUPA conjugates is PSMA-mediated. The lesser antitumor efficacy observed in the group treated with the conjugate 86 (cessation of tumor growth at the dose tested) as compared to the group treated with the free drug 18 (regression of tumor growth at the dose tested) was compensated for by the lower toxicity of the conjugate (FIG. 5). The conjugate 86 is selectively cytotoxic to prostate cancer cells vs. the other cells in the body, resulting in no deaths, whereas free drug 18 resulted in the death of four out of the five animals during the treatment period (FIG. 5). The complete loss of activity in the competition group when the PSMA competitor 28 was used in 10-fold excess of the conjugate 86, as compared to the treated group, suggested that 28 competed with and effectively prevented 86 from binding to PSMA, thereby blocking cellular uptake of 18. This observation indicates (a) sufficient stability of conjugate 86 in solution before cellular uptake, (b) the PSMA-mediated uptake of 86 to tumor cells, and (c) sufficient liability of 86 to enable rapid liberation of free drug 18 following internalization into the malignant cells. In another words, the data implied that the cell-killing effect of conjugate 86 required the presence of an empty PSMA receptor and did not occur by premature extracellular release of the free drug followed by passive diffusion into the diseased cells.

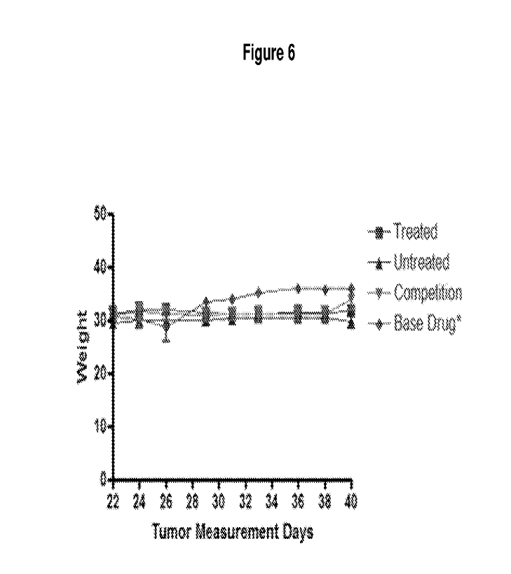

Further, the data in FIG. 4 also documented tumor regression at high doses on the free drug 18. FIG. 6 shows that all live mice in the four groups retained their normal body weights during the treatment period, and indicated that the DUPA-conjugate therapy was well tolerated. In addition, FIG. 6 shows that the base drug is toxic, which resulted in the death of 4 mice (out of 5 mice per group) after 3 injections (one week), while the DUPA conjugate, which expressed similar antitumor efficacy, are non-toxic to the animals and much safer as a chemotherapeutic. The greatly reduced toxicity of 86 vs. 18 (FIG. 5) supports the hypothesis that the conjugate would have greater safety and selectivity than the drug itself.

### Example 47

Since PSMA is only expressed at the level of about one million copies per prostate cancer cell (Kularatne, et al. J. Med. Chem. 2010, 53, 7767-7777), only very potent and highly cytotoxic anticancer drugs would be considered for DUPA conjugation so that the low concentrations that get delivered inside prostate cancer cells using PSMA as a shuttle can still be effective. Compound 18, which possesses a reactive hydroxyl group that can be derivatized, exhibits potent Top1 inhibitory activity (+++++) and an excellent cytotoxicity mean-graph midpoint (MGM) GI₅₀ value (87 nM) in the NCI's panel of 60 cancer cell lines.

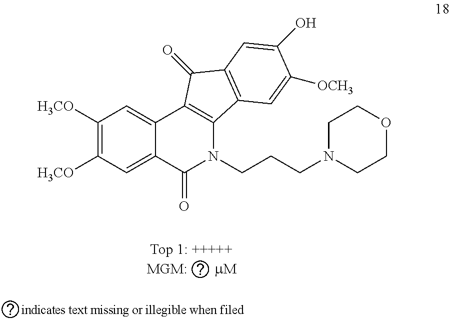 [SMILES: COc1cc2c(cc1O)C(=O)c1c-2n(CCCN2CCOCC2)c(=O)c2cc(OC)c(OC)cc12]

^{a}Top1 inhibitory activity in the Top1-mediated DNA cleavage assay is graded on the following rubric relative to 1 μM camptothecin: 0, no inhibitory activity; +, between 20% and 50% activity; ++, between 50% and 75% activity; +++, between 75% and 95% activity; ++++, equipotent; +++++, more potent. ^{b}The mean-graph midpoint (MGM) is an approximate average of GI₅₀ values across the entire NCI panel of 60 human cancer cell lines successfully tested, where during the MGM calculation, compounds with GI₅₀ values that fall outside the testing range of 0.01-100 μM are assigned values of 0.01 μM and 100 μM.

The biological activities of selected Indenoisoquinolines are listed in Table 1.

TABLE 1
Top1 Inhibitory Activities and Cytotoxicities of Indenoisoquinolines

| Compound | Top1^{a} | MGM^{b} (μM) |
|---|---|---|
| 5 | ++++ | 0.146 |
| 6 | ++++ | 0.047 |
| 7 | ++++ | 0.063 |
| 8 | ++++ | 0.040 |
| 9 | +++ | 0.021 |
| 10 | +++ | 0.152 |
| 11 | ++++ | |
| 12 | ++ | 0.019 |
| 13 | ++ | 0.021 |
| 14 | + | 0.019 |
| 15 | ++++ | 0.016 |
| 16 | ++++ | 0.090 |

^{a}Top1 inhibitory activity.
^{b}Cytotoxicity mean-graph midpoint (average of two determinations).

TABLE 2
Top 1 Inhibitory Activities and Cytotoxicities of Additional Indenoisoquinolines

| Compound | R Group | Top 1^{a} | MGM^{b} (μM) |
|---|---|---|---|
| 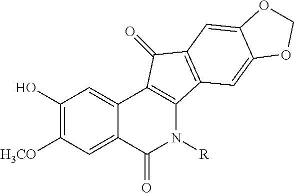 | 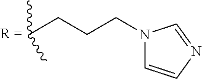 [SMILES: *CCCn1ccnc1] | +++++ | 0.049 |
| 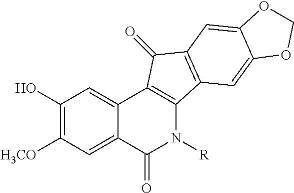 | 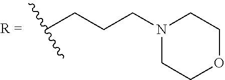 [SMILES: *CCCN1CCOCC1] | ++(+) | 0.412 |
| 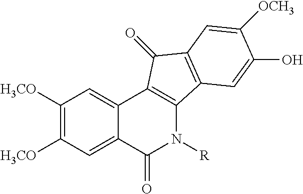 | 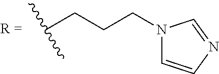 [SMILES: *CCCn1ccnc1] | ++(+) | 41.8 |
| 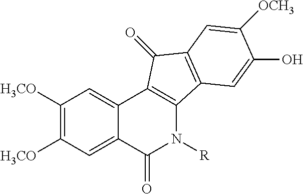 | 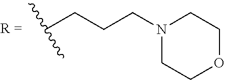 [SMILES: *CCCN1CCOCC1] | +++ | 3.07 |
| 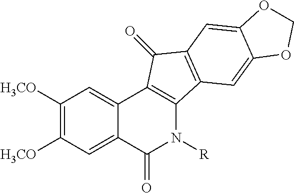 | 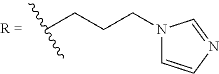 [SMILES: *CCCn1ccnc1] | ++++ | 0.043 |
| 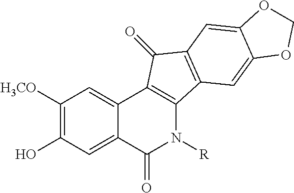 | 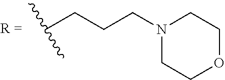 [SMILES: *CCCN1CCOCC1] | +++(+) | 0.056 |
| 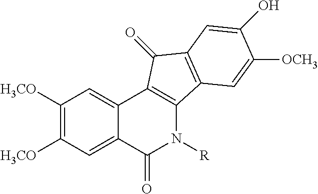 | 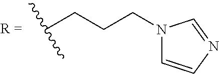 [SMILES: *CCCn1ccnc1] | ++++(+) | 0.055 |
| 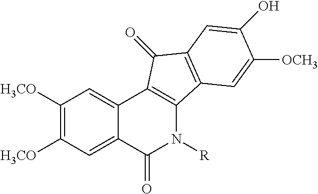 | 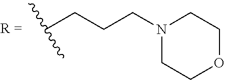 [SMILES: *CCCN1CCOCC1] | +++++ | 0.087 |
| 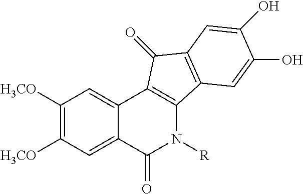 | 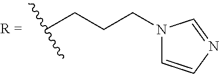 [SMILES: *CCCn1ccnc1] | ++ | 0.224 |
| 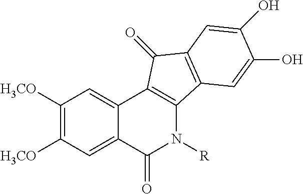 | 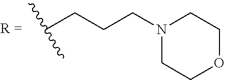 [SMILES: *CCCN1CCOCC1] | ++++ | 0.602 |
| 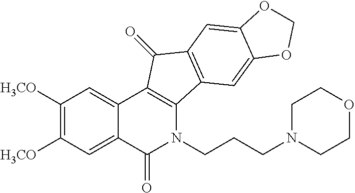 [SMILES: COc1cc2c3c(n(CCCN4CCOCC4)c(=O)c2cc1OC)-c1cc2c(cc1C3=O)OCO2] | | ++++ | 4.64 |
| 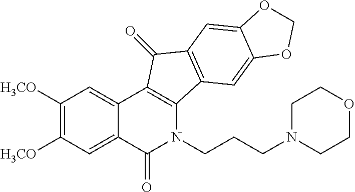 [SMILES: COc1cc2c3c(n(CCCN4CCOCC4)c(=O)c2cc1OC)-c1cc2c(cc1C3=O)OCO2] | | ++++ | 0.079 |
| 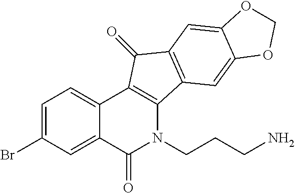 [SMILES: NCCCn1c2c(c3ccc(Br)cc3c1=O)C(=O)c1cc3c(cc1-2)OCO3] | | ++++ | 0.329 |
| 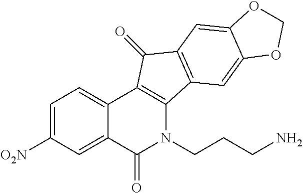 [SMILES: NCCCn1c2c(c3ccc([N+](=O)[O-])cc3c1=O)C(=O)c1cc3c(cc1-2)OCO3] | | ++++ | 0.090 |

### Example 49

### Molecular Modeling

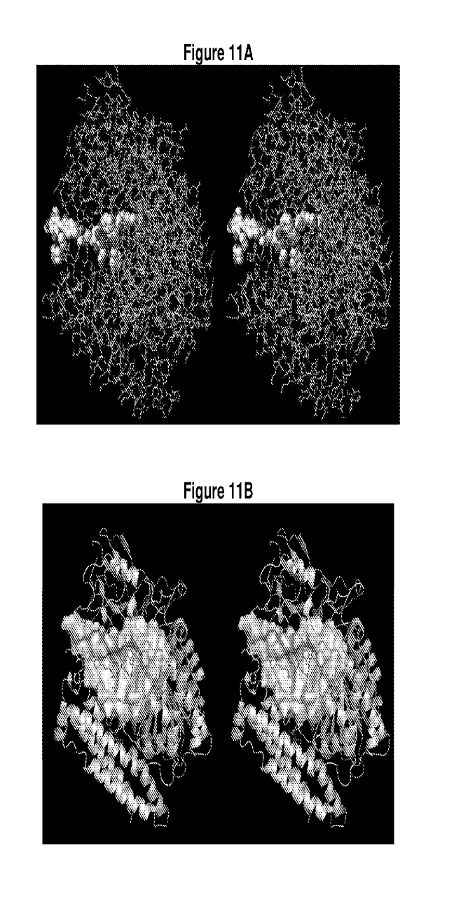

The design of the conjugate 86 was facilitated by molecular modeling of the complex formed between the 86 and PSMA (FIGS. 11A and 11B). The docking and energy minimization procedure used to construct this model can be summarized in the following steps: 1) the conformation of DUPA was energy minimized by Sybyl and then docked into the ligand binding site of PSMA (PDB code 2C6C, with the original ligand GPI-18431 removed) using GOLD 3.0; 2) the conformation of the linker peptide was energy minimized by Sybyl, and then linked to DUPA through a covalent bond and docked to the PSMA binding site using GOLD 3.0; 3) the conformation of the indenoisoquinoline was energy minimized by Sybyl, and then linked to the peptide through a covalent bond and docked to the PSMA binding site using GOLD 3.0; 4) further energy minimization of the resulting conjugate 3-PSMA complex was performed with Sybyl. For the protein, AMBER charges were used. For the ligand, Gasteiger charges were used, and the minimization of the conjugate-PSMA complex was performed with the AMBER7FF99 force field.

According to the molecular model of 86 bound to PSMA displayed in FIGS. 11A and 11B, the DUPA fragment on the right side of the conjugate and the connected polymethylene linker occupy an L-shaped tunnel shown here in the center of the protein. The conjugate structure emerges from the tunnel at the level of the two phenylalanines, and the remaining structure pointing to the left has protein on one side but is essentially open to the bulk solvent on the other side (which faces the viewer). Since the disulfide is already exposed to the solvent, future drug molecules that may be attached to the left side of it in FIGS. 11A and 11B can presumably be exchanged without affecting the release mechanism. It is likely that the most challenging problems that will be encountered in future drug design may actually be due to the very practical consideration of having the right solubility characteristics to allow adequate formulation and optimization of bioavailability at the PSMA site of action on the prostate cancer cell. The peptide nature of the linker chain will facilitate modulation of the solubility characteristics of the conjugates through substitution of different amino acid residues, and alternatively, additional nitrogen atoms can be incorporated into the indenoisoquinoline ring system, resulting in greater aqueous solubility while maintaining Top1 inhibitory potency (Kiselev, et al. J. Med. Chem. 2010, 53, 8716-8726; Kiselev, et al. J. Med. Chem. 2011, 54, 6106-6116; Kiselev, et al. J. Med. Chem. 2012, 55, 1682-1697; and Kiselev, et al. J. Org. Chem. 2012, 77, 5167-5172).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A DUPA-Indenoisoquinoline conjugate represented by formula (IB)
DUPA-Linker-RS-Indenoisoquinoline (IB)
wherein
DUPA is a modified or unmodified 2-[3-(1,3-dicarboxypropyl)-ureido]pentanedioic acid;
Linker is a bond, a substituted or unsubstituted alkyl, a peptide, or a peptidoglycan;
Indenoisoquinoline is a substituted or unsubstituted indenoisoquinoline; and
RS is a release segment capable of releasing Indenoisoquinoline within the desired cells,
wherein said release segment is a carbonate segment, a carbamate segment, or an acylhydrazone segment.

2. The DUPA-Indenoisoquinoline conjugate of claim 1, wherein said linker is a peptide.

3. The DUPA-Indenoisoquinoline conjugate of claim 1, wherein said conjugate is represented by formula (II) 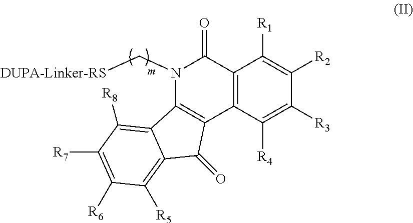
wherein
R₁, R₂, R₃, R₄, R₅, R₆, R₇, and R₈ are each independently H, halo, NR₁₁R₁₂, nitro, C₁₋₅ alkyl, O—C₁₋₃ alkyl, cyano, C₁₋₃ halo alkyl, O—C₁₋₃halo alkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group;
R₁₁ and R₁₂ are each independently H or C₁₋₅ alkyl, wherein C₁₋₅ alkyl is optionally mono- or poly-substituted with substituents independently selected from halo, OH, O—C₁₋₃ alkyl, amino, C₁₋₃ alkylamino, and di-C₁₋₃ alkylamino; or R₁₁ and R₁₂, together with the nitrogen atom to which they are attached, form a 4-7 membered cycloheteroalkyl or heteroaryl; and
m is 0-5.

4. (canceled)

5. (canceled)

6. The DUPA-Indenoisoquinoline conjugate of claim 3, wherein said conjugate is represented by formula (V)

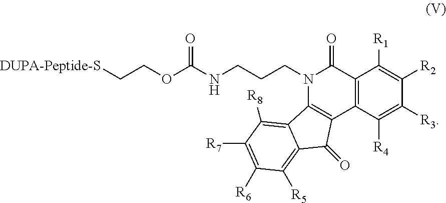

7-18. (canceled)

19. The DUPA-Indenoisoquinoline conjugate of claim 1, wherein said conjugate is represented by formula (III) 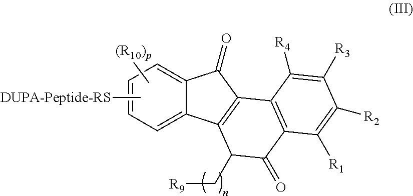
wherein
R₁, R₂, R₃, R₄, and R₁₀ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group;
R₉ is H, halo, O—C₁₋₅ alkyl, NR₁₁R₁₂, nitro, C₃₋₆ cycloalkyl, or C₃₋₈ cycloheteroalkyl;
R₁₁ and R₁₂ are each independently H or C₁₋₅ alkyl, wherein C₁₋₅ alkyl is optionally mono- or poly-substituted with substituents independently selected from halo, OH, O—C₁₋₃ alkyl, amino, C₁₋₃ alkylamino, and di-C₁₋₃ alkylamino; or R₁₁ and R₁₂, together with the nitrogen atom to which they are attached, form a 4-7 membered cycloheteroalkyl or heteroaryl;
n is 0-5; and
p is 3.

20. The DUPA-Indenoisoquinoline conjugate of claim 19, wherein said conjugate is represented by formula (VI) 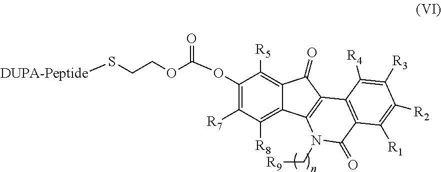
wherein R₅, R₇, and R₈ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group.

21-31. (canceled)

32. The DUPA-Indenoisoquinoline conjugate of claim 19, wherein R₉ is NR₁₁R₁₂.

33. The DUPA-Indenoisoquinoline conjugate of claim 32, wherein R₁₁ and R₁₂ together with the nitrogen atom to which they are attached, form a 4-7 membered cycloheteroalkyl or heteroaryl group.

34. (canceled)

35. (canceled)

36. The DUPA-Indenoisoquinoline conjugate of claim 19, wherein said conjugate is represented by formula (VII) 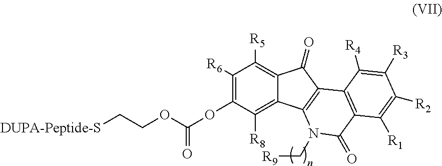
wherein R₅, R₆, and R₈ are each independently H, halo, NR₁₁R₁₂, nitro, C₁₋₃ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group.

37-53. (canceled)

54. The DUPA-Indenoisoquinoline conjugate of claim 1, wherein said conjugate is represented by formula (IV) 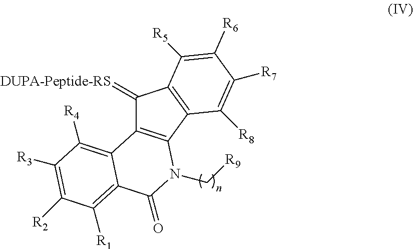
wherein
R₁, R₂, R₃, R₄, R₅, R₆, R₇, and R₈ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group;
R₉ is H, halo, O—C₁₋₅ alkyl, NR₁₁R₁₂, nitro, C₃₋₆ cycloalkyl, or C₃₋₈ cycloheteroalkyl;
R₁₁ and R₁₂ are each independently H or C₁₋₅ alkyl, wherein C₁₋₅ alkyl is optionally mono- or poly-substituted with substituents independently selected from halo, OH, O—C₁₋₃ alkyl, amino, C₁₋₃ alkylamino, and di-C₁₋₃ alkylamino; or R₁₁ and R₁₂, together with the nitrogen atom to which they are attached, form a 4-7 membered cycloheteroalkyl or heteroaryl group; and
n is 0-5.

55. The DUPA-Indenoisoquinoline conjugate of claim 54, wherein said conjugate is represented by formula (VIII)

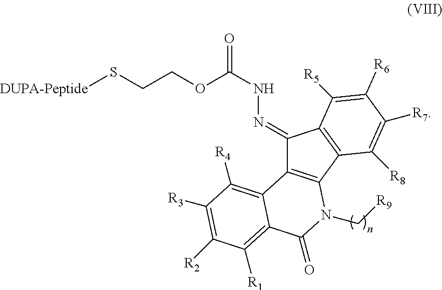

56-74. (canceled)

75. The DUPA-Indenoisoquinoline conjugate of claim 1, wherein said conjugate is represented by formula (IX) 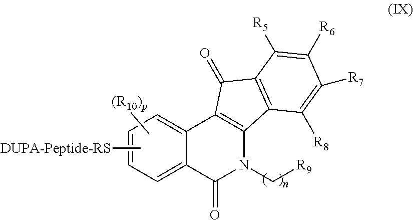
wherein
R₅, R₆, R₇, R₈, and R₁₀ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group;
R₉ is H, halo, O—C₁₋₅ alkyl, NR₁₁R₁₂, nitro, C₃₋₆ cycloalkyl, or C₃₋₈ cycloheteroalkyl;
R₁₁ and R₁₂ are each independently H or C₁₋₅ alkyl, wherein C₁₋₅ alkyl is optionally mono- or poly-substituted with substituents independently selected from halo, OH, O—C₁₋₃ alkyl, amino, C₁₋₃ alkylamino, and di-C₁₋₃ alkylamino; or R₁₁ and R₁₂, together with the nitrogen atom to which they are attached, form a 4-7 membered cycloheteroalkyl or heteroaryl;
n is 0-5; and
p is 3.

76. The DUPA-Indenoisoquinoline conjugate of claim 75, wherein said conjugate is represented by formulas (X) 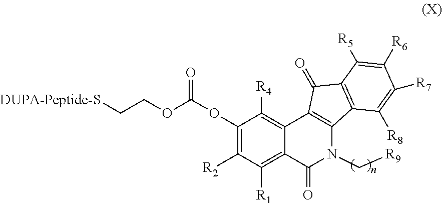
wherein R₁, R₂, and R₄ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group.

77-87. (canceled)

88. The DUPA-Indenoisoquinoline conjugate of claim 75, wherein R₉ is NR₁₁R₁₂.

89. The DUPA-Indenoisoquinoline conjugate of claim 88, wherein R₁₁ and R₁₂ together with the nitrogen atom to which they are attached, form a 4-7 membered cycloheteroalkyl or heteroaryl group.

90. (canceled)

91. (canceled)

92. The DUPA-Indenoisoquinoline conjugate of claim 75, wherein said conjugate is represented by formulas (XI) 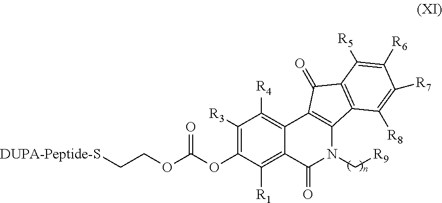
wherein R₁, R₃, and R₄ are each independently H, halo, NR₁₁R₁₂, nitro, C₁₋₅ alkyl, O—C₁₋₃ alkyl, cyano, C₁₋₃ haloalkyl, O—C₁₋₃ haloalkyl, S—C₁₋₃ alkyl, (CO)OR₁₁, (CO)NR₁₁R₁₂, SO₂R₁₁, SO₂NR₁₁R₁₂, or C₃₋₈ cycloheteroalkyl; or two adjacent O—C₁₋₃ alkyl groups, together with the atoms to which they are attached, form a 5-7 membered cycloheteroalkyl group.

93-107. (canceled)

108. The DUPA-Indenoisoquinoline conjugate of claim 1, wherein said conjugate is represented by formulas (XII)-(XX)

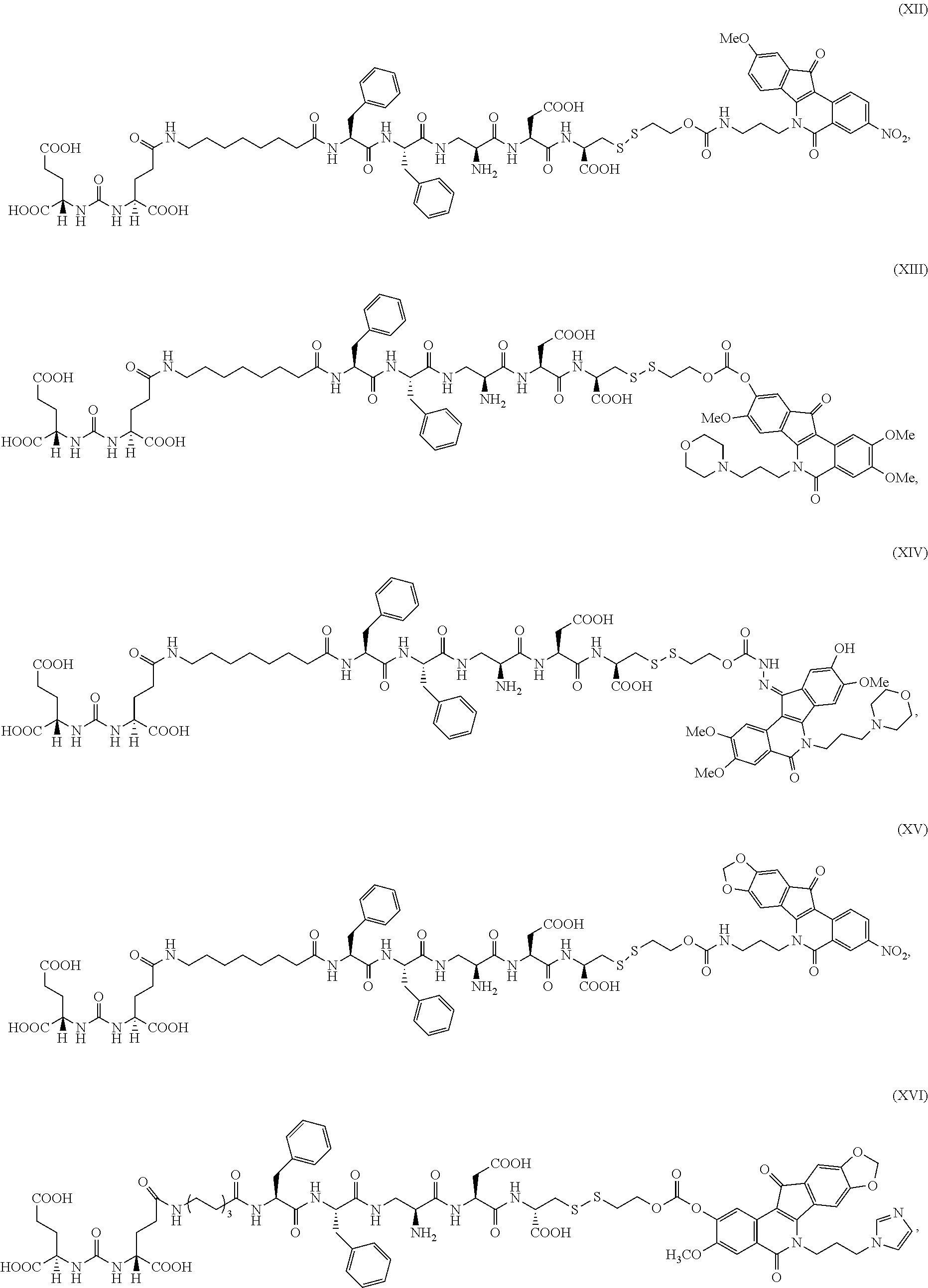

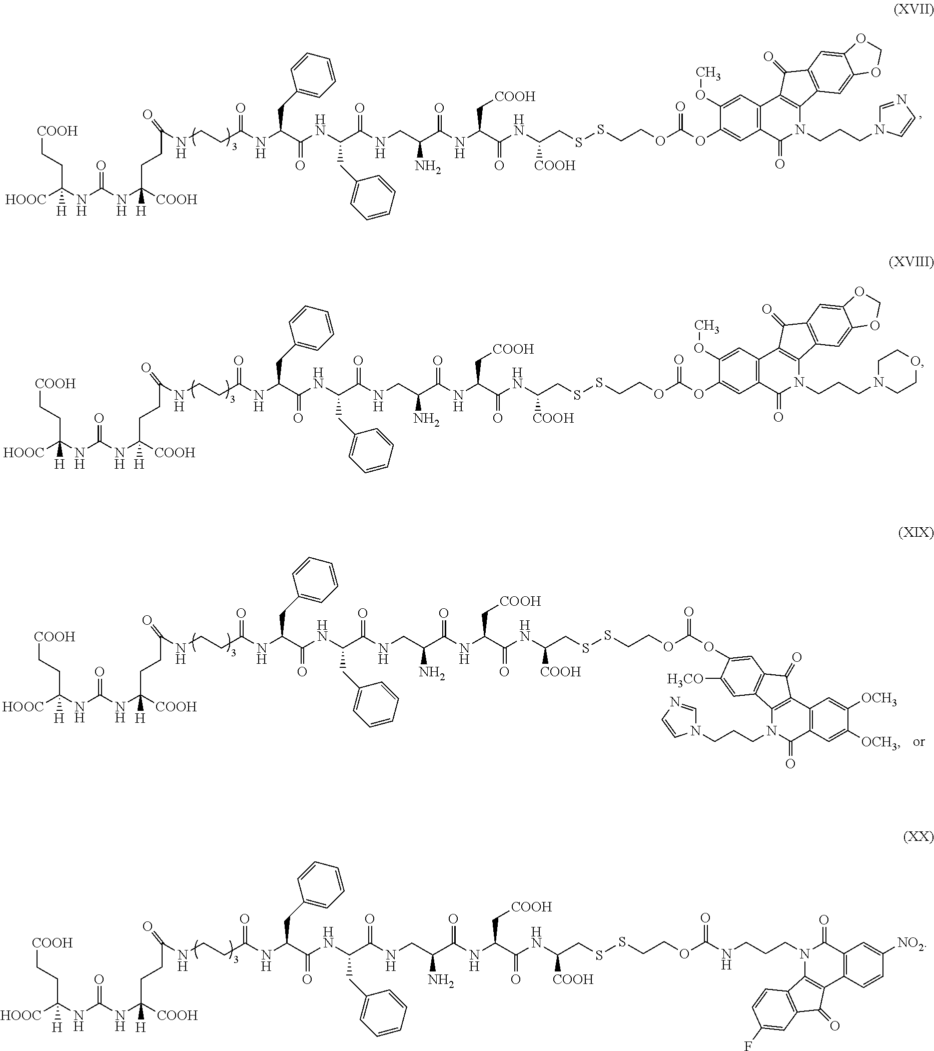

109. A pharmaceutical composition comprising a DUPA-Indenoisoquinoline conjugate of claim 1, and at least one pharmaceutically acceptable carrier.

110. A method of treating cancer in a subject in need thereof, the method comprising administering to said subject a therapeutically effective amount of a DUPA-Indenoisoquinoline conjugate represented by formula (IB) of claim 1.

111. (canceled)

112. The method of claim 110, wherein said cancer is prostate cancer, ovarian cancer, lung cancer, or breast cancer.
